SEQUENCE LISTING (205 sequences)

SEQ ID NO: 1 - protein, 25 aa, synthetic construct
  REGION 1..25: note = PR001046 HFWR1 Chothia
EVQLVETGGGLIQPGGSLRLSCAAS

SEQ ID NO: 2 - protein, 25 aa, synthetic construct
  REGION 1..25: note = PR001848 HFWR1 Chothia, PR011628 HFWR1 Chothia, PR011629 HFWR1 Chothia, PR011630 HFWR1 Chothia, PR011631 HFWR1 Chothia
EVQLLESGGGLVQPGGSLRLSCAAS

SEQ ID NO: 3 - protein, 25 aa, synthetic construct
  REGION 1..25: note = PR003366 HFWR1 Chothia
QVQLVQSGAEVKKPGSSMRVSCKAS

SEQ ID NO: 4 - protein, 25 aa, synthetic construct
  REGION 1..25: note = PR003886 HFWR1 Chothia
EVQLVESGGGLVQPGGSLKLSCAAS

SEQ ID NO: 5 - protein, 25 aa, synthetic construct
  REGION 1..25: note = PR005340 HFWR1 Chothia
EVQLVESGGGLVKPGGSLRLSCAAS

SEQ ID NO: 6 - protein, 25 aa, synthetic construct
  REGION 1..25: note = PR006361 HFWR1 Chothia
EVQLVESGGGPVQAGGSLRLSCAAS

SEQ ID NO: 7 - protein, 25 aa, synthetic construct
  REGION 1..25: note = PR006364 HFWR1 Chothia
RIRLVESGGGLVQAGGSLRLSCAAS

SEQ ID NO: 8 - protein, 25 aa, synthetic construct
  REGION 1..25: note = PR006365 HFWR1 Chothia
EVQLVESGGGLVQAGGSLRLSCAAS

SEQ ID NO: 9 - protein, 25 aa, synthetic construct
  REGION 1..25: note = PR006370 HFWR1 Chothia
EVQLVESGGGLVQPKGSLKLSCAAS

SEQ ID NO: 10 - protein, 25 aa, synthetic construct
  REGION 1..25: note = PR010991 HFWR1 Chothia, PR012876 HFWR1 Chothia, PR012877 HFWR1 Chothia, PR012878 HFWR1 Chothia, PR012879 HFWR1 Chothia, PR012880 HFWR1 Chothia, PR012881 HFWR1 Chothia, PR012882 HFWR1 Chothia, PR012883
RVHLAESGGGLVQAGGSLRLSCAAS

SEQ ID NO: 11 - protein, 25 aa, synthetic construct [sequence omitted]

SEQ ID NO: 12 - protein, 7 aa, synthetic construct
  REGION 1..7: note = PR001046 HCDR1 Chothia
GFTVSDN

SEQ ID NO: 13 - protein, 7 aa, synthetic construct
  REGION 1..7: note = PR001848 HCDR1 Chothia, PR003886 HCDR1 Chothia
GFTFSTY

SEQ ID NO: 14 - protein, 7 aa, synthetic construct
  REGION 1..7: note = PR003366 HCDR1 Chothia
EDTFSSY

SEQ ID NO: 15 - protein, 7 aa, synthetic construct
  REGION 1..7: note = PR005340 HCDR1 Chothia
GFTFSDY

SEQ ID NO: 16 - protein, 7 aa, synthetic construct
  REGION 1..7: note = PR006361 HCDR1 Chothia
GRTYRGY

SEQ ID NO: 17 - protein, 7 aa, synthetic construct
  REGION 1..7: note = PR006364 HCDR1 Chothia, PR011628 HCDR1 Chothia, PR011629 HCDR1 Chothia, PR011630 HCDR1 Chothia, PR011631 HCDR1 Chothia, PR011632 HCDR1 Chothia
GRTSGIP

SEQ ID NO: 18 - protein, 7 aa, synthetic construct
  REGION 1..7: note = PR006365 HCDR1 Chothia
GRTNGIP

SEQ ID NO: 19 - protein, 7 aa, synthetic construct
  REGION 1..7: note = PR006370 HCDR1 Chothia
GFTFNTY

SEQ ID NO: 20 - protein, 7 aa, synthetic construct
  REGION 1..7: note = PR010991 HCDR1 Chothia, PR012876 HCDR1 Chothia, PR012878 HCDR1 Chothia, PR012879 HCDR1 Chothia, PR012880 HCDR1 Chothia, PR012881 HCDR1 Chothia, PR012882 HCDR1 Chothia, PR012883 HCDR1 Chothia, PR012884
GSRLNDN

SEQ ID NO: 21 - protein, 7 aa, synthetic construct
  REGION 1..7: note = PR012877 HCDR1 Chothia
GSWLYDN

SEQ ID NO: 22 - protein, 7 aa, synthetic construct
  REGION 1..7: note = PR012889 HCDR1 Chothia
GSRLRDN

SEQ ID NO: 23 - protein, 19 aa, synthetic construct
  REGION 1..19: note = PR001046 HFWR2 Chothia
YMTWVRQAPGKGLEWVSVI

SEQ ID NO: 24 - protein, 19 aa, synthetic construct
  REGION 1..19: note = PR001848 HFWR2 Chothia
AMNWVRQAPGKGLEWVSRI

SEQ ID NO: 25 - protein, 19 aa, synthetic construct
  REGION 1..19: note = PR003366 HFWR2 Chothia
AISWVRQAPGQGLEWMGGT

SEQ ID NO: 26 - protein, 19 aa, synthetic construct
  REGION 1..19: note = PR003886 HFWR2 Chothia
AMNWVRQASGKGLEWVGRI

SEQ ID NO: 27 - protein, 19 aa, synthetic construct
  REGION 1..19: note = PR005340 HFWR2 Chothia
YMSWIRQAPGKGLEWISYI

SEQ ID NO: 28 - protein, 19 aa, synthetic construct
  REGION 1..19: note = PR006361 HFWR2 Chothia
SMGWFRQAPGKEREFVAAI

SEQ ID NO: 29 - protein, 19 aa, synthetic construct
  REGION 1..19: note = PR006364 HFWR2 Chothia, PR006365 HFWR2 Chothia
AMAWFRQAPGKEREFVAMA

SEQ ID NO: 30 - protein, 19 aa, synthetic construct
  REGION 1..19: note = PR006370 HFWR2 Chothia
AMNWVRQAPGKGLEWVARI

SEQ ID NO: 31 - protein, 19 aa, synthetic construct
  REGION 1..19: note = PR010991 HFWR2 Chothia, PR012876 HFWR2 Chothia, PR012877 HFWR2 Chothia, PR012878 HFWR2 Chothia, PR012879 HFWR2 Chothia, PR012880 HFWR2 Chothia, PR012881 HFWR2 Chothia, PR012882 HFWR2 Chothia, PR012883
AMAWFRQAPGKEREFVGSI

SEQ ID NO: 32 - protein, 19 aa, synthetic construct
  REGION 1..19: note = PR011628 HFWR2 Chothia, PR011631 HFWR2 Chothia
AMSWVRQAPGKGLEWVAMA

SEQ ID NO: 33 - protein, 19 aa, synthetic construct
  REGION 1..19: note = PR011629 HFWR2 Chothia, PR011630 HFWR2 Chothia
AMSWVRQAPGKEREFVAMA

SEQ ID NO: 34 - protein, 19 aa, synthetic construct
  REGION 1..19: note = PR011632 HFWR2 Chothia
AMAWFRQAPGKGLEWVAMA

SEQ ID NO: 35 - protein, 19 aa, synthetic construct
  REGION 1..19: note = PR012889 HFWR2 Chothia
TMAWFRQAPGKEREFVGSI

SEQ ID NO: 36 - protein, 5 aa, synthetic construct
  REGION 1..5: note = PR001046 HCDR2 Chothia
FSGGN

SEQ ID NO: 37 - protein, 8 aa, synthetic construct
  REGION 1..8: note = PR001848 HCDR2 Chothia, PR003886 HCDR2 Chothia, PR006370 HCDR2 Chothia
RSKYNNYA

SEQ ID NO: 38 - protein, 6 aa, synthetic construct
  REGION 1..6: note = PR003366 HCDR2 Chothia
APIFGT

SEQ ID NO: 39 - protein, 6 aa, synthetic construct
  REGION 1..6: note = PR005340 HCDR2 Chothia
SSSGST

SEQ ID NO: 40 - protein, 6 aa, synthetic construct
  REGION 1..6: note = PR006361 HCDR2 Chothia
VWSGGN

SEQ ID NO: 41 - protein, 5 aa, synthetic construct
  REGION 1..5: note = PR006364 HCDR2 Chothia, PR006365 HCDR2 Chothia, PR011628 HCDR2 Chothia, PR011629 HCDR2 Chothia, PR011630 HCDR2 Chothia, PR011631 HCDR2 Chothia, PR011632 HCDR2 Chothia
WSGGG

SEQ ID NO: 42 - protein, 6 aa, synthetic construct
  REGION 1..6: note = PR010991 HCDR2 Chothia, PR012876 HCDR2 Chothia, PR012877 HCDR2 Chothia, PR012878 HCDR2 Chothia, PR012879 HCDR2 Chothia, PR012880 HCDR2 Chothia, PR012881 HCDR2 Chothia, PR012882 HCDR2 Chothia, PR012883
FWTGGN

SEQ ID NO: 43 - protein, 6 aa, synthetic construct
  REGION 1..6: note = PR012890 HCDR2 Chothia
FRTGGN

SEQ ID NO: 44 - protein, 41 aa, synthetic construct
  REGION 1..41: note = PR001046 HFWR3 Chothia
TYYADSVKGRFTISRDNAKNTLYLQMNSLRAEDTALYYCAR

SEQ ID NO: 45 - protein, 41 aa, synthetic construct
  REGION 1..41: note = PR001848 HFWR3 Chothia
TYYADSVKDRFTISRDDSKSTLYLQMNSLRAEDTAVYYCVR

SEQ ID NO: 46 - protein, 41 aa, synthetic construct
  REGION 1..41: note = PR003366 HFWR3 Chothia
TNYAQKFQGRVTITADKSTSTAYMELSSLRSEDTAVYYCAR

SEQ ID NO: 47 - protein, 41 aa, synthetic construct
  REGION 1..41: note = PR003886 HFWR3 Chothia
TYYADSVKDRFTISRDDSKNTAYLQMNSLKTEDTAVYYCTR

SEQ ID NO: 48 - protein, 41 aa, synthetic construct
  REGION 1..41: note = PR005340 HFWR3 Chothia
IHYADSVKGRFTISRDNAKNSLYLQMNSLRAEDTAIYYCAR

SEQ ID NO: 49 - protein, 41 aa, synthetic construct
  REGION 1..41: note = PR006361 HFWR3 Chothia
TYYEDSVKGRFTISRDNAKNTMYLQMTSLKPEDSATYYCAA

SEQ ID NO: 50 - protein, 41 aa, synthetic construct
  REGION 1..41: note = PR006364 HFWR3 Chothia
TIYGDSVKNRFTISRDNTQNTIFLQMNSLKPEDTALYYCAA

SEQ ID NO: 51 - protein, 41 aa, synthetic construct
  REGION 1..41: note = PR006365 HFWR3 Chothia
TIYGDSVKNRFTISRNNAQNTISLQMSSLRPEDTALYFCAA

SEQ ID NO: 52 - protein, 41 aa, synthetic construct
  REGION 1..41: note = PR006370 HFWR3 Chothia
TYYADSVKDRFTISRDDSQSILYLQMNNLKTEDTAMYYCVR

SEQ ID NO: 53 - protein, 41 aa, synthetic construct
  REGION 1..41: note = PR010991 HFWR3 Chothia, PR012876 HFWR3 Chothia, PR012877 HFWR3 Chothia, PR012878 HFWR3 Chothia, PR012879 HFWR3 Chothia, PR012880 HFWR3 Chothia, PR012881 HFWR3 Chothia, PR012882 HFWR3 Chothia, PR012883
TYYADAVKGRLTISRDNAKNALYLQMNSLKPEDTAVYYCAA

SEQ ID NO: 54 - protein, 41 aa, synthetic construct
  REGION 1..41: note = PR011628 HFWR3 Chothia, PR011629 HFWR3 Chothia, PR011630 HFWR3 Chothia, PR011631 HFWR3 Chothia
TIYADSVKGRFTISRDNSKNTLYLQMNSLRAEDTAVYYCAA

SEQ ID NO: 55 - protein, 41 aa, synthetic construct
  REGION 1..41: note = PR011632 HFWR3 Chothia
TIYADSVKGRFTISRDNSKNTLYLQMNSLRAEDTAVYYCAR

SEQ ID NO: 56 - protein, 41 aa, synthetic construct
  REGION 1..41: note = PR012890 HFWR3 Chothia
AYYADAVKGRLTISRDNAKNALYLQMNSLKPEDTAVYYCAA

SEQ ID NO: 57 - protein, 14 aa, synthetic construct
  REGION 1..14: note = PR001046 HCDR3 Chothia
RNYDDTRGTDVFDI

SEQ ID NO: 58 - protein, 14 aa, synthetic construct
  REGION 1..14: note = PR001848 HCDR3 Chothia, PR003886 HCDR3 Chothia, PR006370 HCDR3 Chothia
HGNFGNSYVSWFAY

SEQ ID NO: 59 - protein, 7 aa, synthetic construct
  REGION 1..7: note = PR003366 HCDR3 Chothia
GGPYFDY

SEQ ID NO: 60 - protein, 16 aa, synthetic construct
  REGION 1..16: note = PR005340 HCDR3 Chothia
DIPSSSSDWVSLQFDY

SEQ ID NO: 61 - protein, 15 aa, synthetic construct
  REGION 1..15: note = PR006361 HCDR3 Chothia
KIRPYIFKIAGQYDY

SEQ ID NO: 62 - protein, 13 aa, synthetic construct
  REGION 1..13: note = PR006364 HCDR3 Chothia, PR011628 HCDR3 Chothia, PR011629 HCDR3 Chothia, PR011632 HCDR3 Chothia
GRSTSNRADQYNY

SEQ ID NO: 63 - protein, 13 aa, synthetic construct
  REGION 1..13: note = PR006365 HCDR3 Chothia
GRTTSNKADHYNY

SEQ ID NO: 64 - protein, 21 aa, synthetic construct
  REGION 1..21: note = PR010991 HCDR3 Chothia, PR012877 HCDR3 Chothia, PR012889 HCDR3 Chothia, PR012890 HCDR3 Chothia
SRVVWYYCSGYGCSRAGDYVY

SEQ ID NO: 65 - protein, 13 aa, synthetic construct
  REGION 1..13: note = PR011630 HCDR3 Chothia, PR011631 HCDR3 Chothia
GRSTSNYADQYNY

SEQ ID NO: 66 - protein, 21 aa, synthetic construct
  REGION 1..21: note = PR012876 HCDR3 Chothia
SRVVWYYCSGYGCSRAADYVY

SEQ ID NO: 67 - protein, 21 aa, synthetic construct
  REGION 1..21: note = PR012878 HCDR3 Chothia
SRVVWYYCTRYGCSRPGDYVY

SEQ ID NO: 68 - protein, 21 aa, synthetic construct
  REGION 1..21: note = PR012879 HCDR3 Chothia
SRVVWYYCSAYGCSGAGNYVY

SEQ ID NO: 69 - protein, 21 aa, synthetic construct
  REGION 1..21: note = PR012880 HCDR3 Chothia
SRVVWYYCYRYGCSRGGDYVY

SEQ ID NO: 70 - protein, 21 aa, synthetic construct
  REGION 1..21: note = PR012881 HCDR3 Chothia
SRVVWYYCSRYGCSRSGDYAY

SEQ ID NO: 71 - protein, 21 aa, synthetic construct
  REGION 1..21: note = PR012882 HCDR3 Chothia
SRVVWYYCSGYGCSRAVDYVY

SEQ ID NO: 72 - protein, 21 aa, synthetic construct
  REGION 1..21: note = PR012883 HCDR3 Chothia
SRVVWYYCASYGCSRPVHYVY

SEQ ID NO: 73 - protein, 21 aa, synthetic construct
  REGION 1..21: note = PR012884 HCDR3 Chothia
SRVVWYYCSGYGCSHARDYIY

SEQ ID NO: 74 - protein, 21 aa, synthetic construct
  REGION 1..21: note = PR012885 HCDR3 Chothia
SRVVWYYCSGYGCSRAHDYAY

SEQ ID NO: 75 - protein, 21 aa, synthetic construct
  REGION 1..21: note = PR012886 HCDR3 Chothia
SRVVWYYCSGYGCSRPRDYVY

SEQ ID NO: 76 - protein, 21 aa, synthetic construct
  REGION 1..21: note = PR012887 HCDR3 Chothia
SRVVWYYCSGYGCSRSPDYVY

SEQ ID NO: 77 - protein, 21 aa, synthetic construct
  REGION 1..21: note = PR012888 HCDR3 Chothia
SRVVWYYCSGYGCSHAANYRY

SEQ ID NO: 78 - protein, 21 aa, synthetic construct
  REGION 1..21: note = PR012891 HCDR3 Chothia
SRVVWYYCAGYGCSRAGNYVY

SEQ ID NO: 79 - protein, 21 aa, synthetic construct
  REGION 1..21: note = PR012892 HCDR3 Chothia
SRVVWYYCSGYGCSRTGDYVY

SEQ ID NO: 80 - protein, 11 aa, synthetic construct
  REGION 1..11: note = PR001046 HFWR4 Chothia
WGQGTMVTVSS

SEQ ID NO: 81 - protein, 11 aa, synthetic construct
  REGION 1..11: note = PR001848 HFWR4 Chothia, PR003366 HFWR4 Chothia, PR003886 HFWR4 Chothia, PR005340 HFWR4 Chothia, PR011628 HFWR4 Chothia, PR011631 HFWR4 Chothia, PR011632 HFWR4 Chothia
WGQGTLVTVSS

SEQ ID NO: 82 - protein, 11 aa, synthetic construct
  REGION 1..11: note = PR006361 HFWR4 Chothia, PR006364 HFWR4 Chothia, PR006365 HFWR4 Chothia, PR010991 HFWR4 Chothia, PR011629 HFWR4 Chothia, PR011630 HFWR4 Chothia, PR012876 HFWR4 Chothia, PR012877 HFWR4 Chothia, PR012878
WGQGTQVTVSS

SEQ ID NO: 83 - protein, 11 aa, synthetic construct
  REGION 1..11: note = PR006370 HFWR4 Chothia
WGQGTLVTVSA

SEQ ID NO: 84 - protein, 22 aa, synthetic construct
  REGION 1..22: note = PR001848 LFWR1 Chothia, PR003886 LFWR1 Chothia
QAVVTQEPSLTVSPGGTVTLTC

SEQ ID NO: 85 - protein, 23 aa, synthetic construct
  REGION 1..23: note = PR003366 LFWR1 Chothia
EIVMTQSPASLSVSPGERATLSC

SEQ ID NO: 86 - protein, 22 aa, synthetic construct
  REGION 1..22: note = PR006370 LFWR1 Chothia
QAVVTQESALTTSPGETVTLTC

SEQ ID NO: 87 - protein, 14 aa, synthetic construct
  REGION 1..14: note = PR001848 LCDR1 Chothia, PR003886 LCDR1 Chothia, PR006370 LCDR1 Chothia
RSSTGAVTTSNYAN

SEQ ID NO: 88 - protein, 11 aa, synthetic construct
  REGION 1..11: note = PR003366 LCDR1 Chothia
RASQSVSSNLA

SEQ ID NO: 89 - protein, 15 aa, synthetic construct
  REGION 1..15: note = PR001848 LFWR2 Chothia, PR003886 LFWR2 Chothia
WVQQKPGQAPRGLIG

SEQ ID NO: 90 - protein, 15 aa, synthetic construct
  REGION 1..15: note = PR003366 LFWR2 Chothia
WYQQKPGQAPRLLIY

SEQ ID NO: 91 - protein, 15 aa, synthetic construct
  REGION 1..15: note = PR006370 LFWR2 Chothia
WVQEKPDHLFTGLIG

SEQ ID NO: 92 - protein, 7 aa, synthetic construct
  REGION 1..7: note = PR001848 LCDR2 Chothia, PR003886 LCDR2 Chothia, PR006370 LCDR2 Chothia
GTNKRAP

SEQ ID NO: 93 - protein, 7 aa, synthetic construct
  REGION 1..7: note = PR003366 LCDR2 Chothia
GASTRAT

SEQ ID NO: 94 - protein, 32 aa, synthetic construct
  REGION 1..32: note = PR001848 LFWR3 Chothia, PR003886 LFWR3 Chothia
WTPARFSGSLLGDKAALTLLGAQPEDEAEYFC

SEQ ID NO: 95 - protein, 32 aa, synthetic construct
  REGION 1..32: note = PR003366 LFWR3 Chothia
GIPARVSGGGSGTEFTLTISSLQSEDFAVYYC

SEQ ID NO: 96 - protein, 32 aa, synthetic construct
  REGION 1..32: note = PR006370 LFWR3 Chothia
GVPARFSGSLIGDKAALTITGAQTEDEAIYFC

SEQ ID NO: 97 - protein, 9 aa, synthetic construct
  REGION 1..9: note = PR001848 LCDR3 Chothia, PR003886 LCDR3 Chothia, PR006370 LCDR3 Chothia
ALWYSNLWV

SEQ ID NO: 98 - protein, 9 aa, synthetic construct
  REGION 1..9: note = PR003366 LCDR3 Chothia
QQYKNWPFT

SEQ ID NO: 99 - protein, 10 aa, synthetic construct
  REGION 1..10: note = PR001848 LFWR4 Chothia, PR003886 LFWR4 Chothia, PR006370 LFWR4 Chothia
FGGGTKLTVL

SEQ ID NO: 100 - protein, 10 aa, synthetic construct
  REGION 1..10: note = PR003366 LFWR4 Chothia
FGPGTKLEIK

SEQ ID NO: 101 - protein, 122 aa, synthetic construct
  REGION 1..122: note = PR001046 VH
EVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSS

SEQ ID NO: 102 - protein, 125 aa, synthetic construct
  REGION 1..125: note = PR001848 VH
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSS

SEQ ID NO: 103 - protein, 116 aa, synthetic construct
  REGION 1..116: note = PR003366 VH
QVQLVQSGAEVKKPGSSMRVSCKASEDTFSSYAISWVRQAPGQGLEWMGGTAPIFGTTNYAQKFQGRVTITADKSTSTAYMELSSLRSEDTAVYYCARGGPYFDYWGQGTLVTVSS

SEQ ID NO: 104 - protein, 125 aa, synthetic construct
  REGION 1..125: note = PR003886 VH
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 105 - protein, 125 aa, synthetic construct
  REGION 1..125: note = PR005340 VH
EVQLVESGGGLVKPGGSLRLSCAASGFTFSDYYMSWIRQAPGKGLEWISYISSSGSTIHYADSVKGRFTISRDNAKNSLYLQMNSLRAEDTAIYYCARDIPSSSSDWVSLQFDYWGQGTLVTVSS

SEQ ID NO: 106 - protein, 124 aa, synthetic construct
  REGION 1..124: note = PR006361 VH
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 107 - protein, 121 aa, synthetic construct
  REGION 1..121: note = PR006364 VH
RIRLVESGGGLVQAGGSLRLSCAASGRTSGIPAMAWFRQAPGKEREFVAMAWSGGGTIYGDSVKNRFTISRDNTQNTIFLQMNSLKPEDTALYYCAAGRSTSNRADQYNYWGQGTQVTVSS

SEQ ID NO: 108 - protein, 121 aa, synthetic construct
  REGION 1..121: note = PR006365 VH
EVQLVESGGGLVQAGGSLRLSCAASGRTNGIPAMAWFRQAPGKEREFVAMAWSGGGTIYGDSVKNRFTISRNNAQNTISLQMSSLRPEDTALYFCAAGRTTSNKADHYNYWGQGTQVTVSS

SEQ ID NO: 109 - protein, 125 aa, synthetic construct
  REGION 1..125: note = PR006370 VH
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 110 - protein, 130 aa, synthetic construct
  REGION 1..130: note = PR010991 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSS

SEQ ID NO: 111 - protein, 121 aa, synthetic construct
  REGION 1..121: note = PR011628 VH
EVQLLESGGGLVQPGGSLRLSCAASGRTSGIPAMSWVRQAPGKGLEWVAMAWSGGGTIYADSVKGRFTISRDNSKNTLYLQMNSLRAEDTAVYYCAAGRSTSNRADQYNYWGQGTLVTVSS

SEQ ID NO: 112 - protein, 121 aa, synthetic construct
  REGION 1..121: note = PR011629 VH
EVQLLESGGGLVQPGGSLRLSCAASGRTSGIPAMSWVRQAPGKEREFVAMAWSGGGTIYADSVKGRFTISRDNSKNTLYLQMNSLRAEDTAVYYCAAGRSTSNRADQYNYWGQGTQVTVSS

SEQ ID NO: 113 - protein, 121 aa, synthetic construct
  REGION 1..121: note = PR011630 VH
EVQLLESGGGLVQPGGSLRLSCAASGRTSGIPAMSWVRQAPGKEREFVAMAWSGGGTIYADSVKGRFTISRDNSKNTLYLQMNSLRAEDTAVYYCAAGRSTSNYADQYNYWGQGTQVTVSS

SEQ ID NO: 114 - protein, 121 aa, synthetic construct
  REGION 1..121: note = PR011631 VH
EVQLLESGGGLVQPGGSLRLSCAASGRTSGIPAMSWVRQAPGKGLEWVAMAWSGGGTIYADSVKGRFTISRDNSKNTLYLQMNSLRAEDTAVYYCAAGRSTSNYADQYNYWGQGTLVTVSS

SEQ ID NO: 115 - protein, 121 aa, synthetic construct
  REGION 1..121: note = PR011632 VH
EVQLVESGGGLVQPGGSLRLSCAASGRTSGIPAMAWFRQAPGKGLEWVAMAWSGGGTIYADSVKGRFTISRDNSKNTLYLQMNSLRAEDTAVYYCARGRSTSNRADQYNYWGQGTLVTVSS

SEQ ID NO: 116 - protein, 130 aa, synthetic construct
  REGION 1..130: note = PR012876 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAADYVYWGQGTQVTVSS

SEQ ID NO: 117 - protein, 130 aa, synthetic construct
  REGION 1..130: note = PR012877 VH
RVHLAESGGGLVQAGGSLRLSCAASGSWLYDNAMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSS

SEQ ID NO: 118 - protein, 130 aa, synthetic construct
  REGION 1..130: note = PR012878 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCTRYGCSRPGDYVYWGQGTQVTVSS

SEQ ID NO: 119 - protein, 130 aa, synthetic construct
  REGION 1..130: note = PR012879 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSAYGCSGAGNYVYWGQGTQVTVSS

SEQ ID NO: 120 - protein, 130 aa, synthetic construct
  REGION 1..130: note = PR012880 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCYRYGCSRGGDYVYWGQGTQVTVSS

SEQ ID NO: 121 - protein, 130 aa, synthetic construct
  REGION 1..130: note = PR012881 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSRYGCSRSGDYAYWGQGTQVTVSS

SEQ ID NO: 122 - protein, 130 aa, synthetic construct
  REGION 1..130: note = PR012882 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAVDYVYWGQGTQVTVSS

SEQ ID NO: 123 - protein, 130 aa, synthetic construct
  REGION 1..130: note = PR012883 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCASYGCSRPVHYVYWGQGTQVTVSS

SEQ ID NO: 124 - protein, 130 aa, synthetic construct
  REGION 1..130: note = PR012884 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSHARDYIYWGQGTQVTVSS

SEQ ID NO: 125 - protein, 130 aa, synthetic construct
  REGION 1..130: note = PR012885 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAHDYAYWGQGTQVTVSS

SEQ ID NO: 126 - protein, 130 aa, synthetic construct
  REGION 1..130: note = PR012886 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRPRDYVYWGQGTQVTVSS

SEQ ID NO: 127 - protein, 130 aa, synthetic construct
  REGION 1..130: note = PR012887 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRSPDYVYWGQGTQVTVSS

SEQ ID NO: 128 - protein, 130 aa, synthetic construct
  REGION 1..130: note = PR012888 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSHAANYRYWGQGTQVTVSS

SEQ ID NO: 129 - protein, 130 aa, synthetic construct
  REGION 1..130: note = PR012889 VH
RVHLAESGGGLVQAGGSLRLSCAASGSRLRDNTMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSS

SEQ ID NO: 130 - protein, 130 aa, synthetic construct
  REGION 1..130: note = PR012890 VH
RVHLAESGGGLVQAGGSLRLSCAASGSRLNDNAMAWFRQAPGKEREFVGSIFRTGGNAYYADAVKGRLTISRDNAKNALYLQMNSLKPEDTAVYYCAASRVVWYYCSGYGCSRAGDYVYWGQGTQVTVSS

SEQ ID NO: 131 - protein, 130 aa, synthetic construct
  REGION 1..130: note = PR012891 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCAGYGCSRAGNYVYWGQGTQVTVSS

SEQ ID NO: 132 - protein, 130 aa, synthetic construct
  REGION 1..130: note = PR012892 VH
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRTGDYVYWGQGTQVTVSS

SEQ ID NO: 133 - protein, 109 aa, synthetic construct
  REGION 1..109: note = PR001848 VL, PR003886 VL
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVL

SEQ ID NO: 134 - protein, 107 aa, synthetic construct
  REGION 1..107: note = PR003366 VL
EIVMTQSPASLSVSPGERATLSCRASQSVSSNLAWYQQKPGQAPRLLIYGASTRATGIPARVSGGGSGTEFTLTISSLQSEDFAVYYCQQYKNWPFTFGPGTKLEIK

SEQ ID NO: 135 - protein, 109 aa, synthetic construct
  REGION 1..109: note = PR006370 VL
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 136 - protein, 354 aa, synthetic construct
  REGION 1..354: note = PR001046 HC
EVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 455 aa, synthetic construct
  REGION 1..455: note = PR001848 HC
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 455 aa, synthetic construct
  REGION 1..455: note = PR003886 HC
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 357 aa, synthetic construct
  REGION 1..357: note = PR005340 HC
EVQLVESGGGLVKPGGSLRLSCAASGFTFSDYYMSWIRQAPGKGLEWISYISSSGSTIHYADSVKGRFTISRDNAKNSLYLQMNSLRAEDTAIYYCARDIPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 358 aa, synthetic construct
  REGION 1..358: note = PR006361 HC
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 355 aa, synthetic construct
  REGION 1..355: note = PR006364 HC
RIRLVESGGGLVQAGGSLRLSCAASGRTSGIPAMAWFRQAPGKEREFVAMAWSGGGTIYGDSVKNRFTISRDNTQNTIFLQMNSLKPEDTALYYCAAGRSTSNRADQYNYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 355 aa, synthetic construct
  REGION 1..355: note = PR006365 HC
EVQLVESGGGLVQAGGSLRLSCAASGRTNGIPAMAWFRQAPGKEREFVAMAWSGGGTIYGDSVKNRFTISRNNAQNTISLQMSSLRPEDTALYFCAAGRTTSNKADHYNYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 455 aa, synthetic construct
  REGION 1..455: note = PR006370 HC
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 364 aa, synthetic construct
  REGION 1..364: note = PR010991 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 355 aa, synthetic construct
  REGION 1..355: note = PR011628 HC
EVQLLESGGGLVQPGGSLRLSCAASGRTSGIPAMSWVRQAPGKGLEWVAMAWSGGGTIYADSVKGRFTISRDNSKNTLYLQMNSLRAEDTAVYYCAAGRSTSNRADQYNYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 355 aa, synthetic construct
  REGION 1..355: note = PR011629 HC
EVQLLESGGGLVQPGGSLRLSCAASGRTSGIPAMSWVRQAPGKEREFVAMAWSGGGTIYADSVKGRFTISRDNSKNTLYLQMNSLRAEDTAVYYCAAGRSTSNRADQYNYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 355 aa, synthetic construct
  REGION 1..355: note = PR011630 HC
EVQLLESGGGLVQPGGSLRLSCAASGRTSGIPAMSWVRQAPGKEREFVAMAWSGGGTIYADSVKGRFTISRDNSKNTLYLQMNSLRAEDTAVYYCAAGRSTSNYADQYNYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 355 aa, synthetic construct
  REGION 1..355: note = PR011631 HC
EVQLLESGGGLVQPGGSLRLSCAASGRTSGIPAMSWVRQAPGKGLEWVAMAWSGGGTIYADSVKGRFTISRDNSKNTLYLQMNSLRAEDTAVYYCAAGRSTSNYADQYNYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 355 aa, synthetic construct
  REGION 1..355: note = PR011632 HC
EVQLVESGGGLVQPGGSLRLSCAASGRTSGIPAMAWFRQAPGKGLEWVAMAWSGGGTIYADSVKGRFTISRDNSKNTLYLQMNSLRAEDTAVYYCARGRSTSNRADQYNYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 364 aa, synthetic construct
  REGION 1..364: note = PR012876 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAADYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 364 aa, synthetic construct
  REGION 1..364: note = PR012877 HC
RVHLAESGGGLVQAGGSLRLSCAASGSWLYDNAMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 364 aa, synthetic construct
  REGION 1..364: note = PR012878 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCTRYGCSRPGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 364 aa, synthetic construct
  REGION 1..364: note = PR012879 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSAYGCSGAGNYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 364 aa, synthetic construct
  REGION 1..364: note = PR012880 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCYRYGCSRGGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 364 aa, synthetic construct
  REGION 1..364: note = PR012881 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSRYGCSRSGDYAYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 364 aa, synthetic construct
  REGION 1..364: note = PR012882 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAVDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 364 aa, synthetic construct
  REGION 1..364: note = PR012883 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCASYGCSRPVHYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 364 aa, synthetic construct
  REGION 1..364: note = PR012884 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSHARDYIYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 364 aa, synthetic construct
  REGION 1..364: note = PR012885 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAHDYAYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 364 aa, synthetic construct
  REGION 1..364: note = PR012886 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRPRDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 364 aa, synthetic construct
  REGION 1..364: note = PR012887 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRSPDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 364 aa, synthetic construct
  REGION 1..364: note = PR012888 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSHAANYRYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 364 aa, synthetic construct
  REGION 1..364: note = PR012889 HC
RVHLAESGGGLVQAGGSLRLSCAASGSRLRDNTMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 364 aa, synthetic construct
  REGION 1..364: note = PR012890 HC
RVHLAESGGGLVQAGGSLRLSCAASGSRLNDNAMAWFRQAPGKEREFVGSIFRTGGNAYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 364 aa, synthetic construct
  REGION 1..364: note = PR012891 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCAGYGCSRAGNYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 364 aa, synthetic construct
  REGION 1..364: note = PR012892 HC
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRTGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 215 aa, synthetic construct
  REGION 1..215: note = PR001848 LC, PR003733 Chain, PR003886 LC, PR003899 Chain
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 168 - protein, 215 aa, synthetic construct
  REGION 1..215: note = PR006370 LC
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 169 - protein, 455 aa, synthetic construct
  REGION 1..455: note = PR003733 Chain
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 477 aa, synthetic construct
  REGION 1..477: note = PR003733 Chain, PR003899 Chain, PR006001 Chain, PR006002 Chain, PR006003 Chain
EIVMTQSPASLSVSPGERATLSCRASQSVSSNLAWYQQKPGQAPRLLIYGASTRATGIPARVSGGGSGTEFTLTISSLQSEDFAVYYCQQYKNWPFTFGPGTKLEIKGGGGSGGGGSGGGGSGGGGSQVQLVQSGAEVKKPGSSMRVSCKASEDTFSSYAISWVRQAPGQGLEWMGGTAPIFGTTNYAQKFQGRVTITADKSTSTAYMELSSLRSEDTAVYYCARGGPYFDYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 171 - protein, 455 aa, synthetic construct
  REGION 1..455: note = PR003899 Chain
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 483 aa, synthetic construct
  REGION 1..483: note = PR005860 Chain
RIRLVESGGGLVQAGGSLRLSCAASGRTSGIPAMAWFRQAPGKEREFVAMAWSGGGTIYGDSVKNRFTISRDNTQNTIFLQMNSLKPEDTALYYCAAGRSTSNRADQYNYWGQGTQVTVSSEPKTPKPQPTSGHRGQGGGGSEVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSSASAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 173 - protein, 483 aa, synthetic construct
  REGION 1..483: note = PR005861 Chain
EVQLVESGGGLVQAGGSLRLSCAASGRTNGIPAMAWFRQAPGKEREFVAMAWSGGGTIYGDSVKNRFTISRNNAQNTISLQMSSLRPEDTALYFCAAGRTTSNKADHYNYWGQGTQVTVSSEPKTPKPQPTSGHRGQGGGGSEVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSSASAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 174 - protein, 358 aa, synthetic construct
  REGION 1..358: note = PR006001 Chain
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 355 aa, synthetic construct
  REGION 1..355: note = PR006002 Chain
RIRLVESGGGLVQAGGSLRLSCAASGRTSGIPAMAWFRQAPGKEREFVAMAWSGGGTIYGDSVKNRFTISRDNTQNTIFLQMNSLKPEDTALYYCAAGRSTSNRADQYNYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 355 aa, synthetic construct
  REGION 1..355: note = PR006003 Chain
EVQLVESGGGLVQAGGSLRLSCAASGRTNGIPAMAWFRQAPGKEREFVAMAWSGGGTIYGDSVKNRFTISRNNAQNTISLQMSSLRPEDTALYFCAAGRTTSNKADHYNYWGQGTQVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 364 aa, synthetic construct
  REGION 1..364: note = PR008551 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 477 aa, synthetic construct
  REGION 1..477: note = PR008551 Chain, PR010982 Chain, PR010983 Chain, PR010984 Chain, PR010985 Chain, PR010987 Chain, PR012561 Chain, PR012562 Chain, PR012563 Chain, PR012564 Chain, PR012565 Chain, PR012566 Chain, PR012567
EIVMTQSPASLSVSPGERATLSCRASQSVSSNLAWYQQKPGQAPRLLIYGASTRATGIPARVSGGGSGTEFTLTISSLQSEDFAVYYCQQYKNWPFTFGPGTKLEIKGGGGSGGGGSGGGGSGGGGSQVQLVQSGAEVKKPGSSMRVSCKASEDTFSSYAISWVRQAPGQGLEWMGGTAPIFGTTNYAQKFQGRVTITADKSTSTAYMELSSLRSEDTAVYYCARGGPYFDYWGQGTLVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 179 - protein, 355 aa, synthetic construct
  REGION 1..355: note = PR010982 Chain
EVQLLESGGGLVQPGGSLRLSCAASGRTSGIPAMSWVRQAPGKGLEWVAMAWSGGGTIYADSVKGRFTISRDNSKNTLYLQMNSLRAEDTAVYYCAAGRSTSNRADQYNYWGQGTLVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 355 aa, synthetic construct
  REGION 1..355: note = PR010983 Chain
EVQLLESGGGLVQPGGSLRLSCAASGRTSGIPAMSWVRQAPGKEREFVAMAWSGGGTIYADSVKGRFTISRDNSKNTLYLQMNSLRAEDTAVYYCAAGRSTSNRADQYNYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 355 aa, synthetic construct
  REGION 1..355: note = PR010984 Chain
EVQLLESGGGLVQPGGSLRLSCAASGRTSGIPAMSWVRQAPGKEREFVAMAWSGGGTIYADSVKGRFTISRDNSKNTLYLQMNSLRAEDTAVYYCAAGRSTSNYADQYNYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 355 aa, synthetic construct
  REGION 1..355: note = PR010985 Chain
EVQLLESGGGLVQPGGSLRLSCAASGRTSGIPAMSWVRQAPGKGLEWVAMAWSGGGTIYADSVKGRFTISRDNSKNTLYLQMNSLRAEDTAVYYCAAGRSTSNYADQYNYWGQGTLVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 355 aa, synthetic construct
  REGION 1..355: note = PR010987 Chain
EVQLVESGGGLVQPGGSLRLSCAASGRTSGIPAMAWFRQAPGKGLEWVAMAWSGGGTIYADSVKGRFTISRDNSKNTLYLQMNSLRAEDTAVYYCARGRSTSNRADQYNYWGQGTLVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 409 aa, synthetic construct
  REGION 1..409: note = PR011870 Chain
EVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDIPSSSSDWVDLQFDYWGQGTLVTVSSGGGGSGGGGSGGGGSEVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDIPSSSSDWVDLQFDYWGQGTLVTVSSGGGGSGGGGSGGGGSRIRLVESGGGLVQAGGSLRLSCAASGRTSGIPAMAWFRQAPGKEREFVAMAWSGGGTIYGDSVKNRFTISRDNTQNTIFLQMNSLKPEDTALYYCAAGRSTSNRADQYNYWGQGTQVTVSSASHHHHHH

SEQ ID NO: 185 - protein, 364 aa, synthetic construct
  REGION 1..364: note = PR012561 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAADYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 364 aa, synthetic construct
  REGION 1..364: note = PR012562 Chain
RVHLAESGGGLVQAGGSLRLSCAASGSWLYDNAMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 364 aa, synthetic construct
  REGION 1..364: note = PR012563 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCTRYGCSRPGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 364 aa, synthetic construct
  REGION 1..364: note = PR012564 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSAYGCSGAGNYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 364 aa, synthetic construct
  REGION 1..364: note = PR012565 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCYRYGCSRGGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 364 aa, synthetic construct
  REGION 1..364: note = PR012566 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSRYGCSRSGDYAYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 364 aa, synthetic construct
  REGION 1..364: note = PR012567 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAVDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 364 aa, synthetic construct
  REGION 1..364: note = PR012568 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCASYGCSRPVHYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 364 aa, synthetic construct
  REGION 1..364: note = PR012569 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSHARDYIYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 364 aa, synthetic construct
  REGION 1..364: note = PR012644 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRAHDYAYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 364 aa, synthetic construct
  REGION 1..364: note = PR012645 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRPRDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 364 aa, synthetic construct
  REGION 1..364: note = PR012646 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRSPDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 364 aa, synthetic construct
  REGION 1..364: note = PR012647 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSHAANYRYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 364 aa, synthetic construct
  REGION 1..364: note = PR012683 Chain
RVHLAESGGGLVQAGGSLRLSCAASGSRLRDNTMAWFRQAPGKEREFVGSIFWTGGNTYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 364 aa, synthetic construct
  REGION 1..364: note = PR012684 Chain
RVHLAESGGGLVQAGGSLRLSCAASGSRLNDNAMAWFRQAPGKEREFVGSIFRTGGNAYYADAVKGRLTISRDNAKNALYLQMNSLKPEDTAVYYCAASRVVWYYCSGYGCSRAGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 364 aa, synthetic construct
  REGION 1..364: note = PR012685 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCAGYGCSRAGNYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 364 aa, synthetic construct
  REGION 1..364: note = PR012686 Chain
RVHLAESGGGLVQAGGSLRLSCAASGSRLNDNAMAWFRQAPGKEREFVGSIFWTGGNTYYADAVKGRLTISRDNAKNALYLQMNSLKPEDTAVYYCAASRVVWYYCSGYGCSRTGDYVYWGQGTQVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 480 aa, synthetic construct
  REGION 1..480: note = PR003366 SCFV
EIVMTQSPASLSVSPGERATLSCRASQSVSSNLAWYQQKPGQAPRLLIYGASTRATGIPARVSGGGSGTEFTLTISSLQSEDFAVYYCQQYKNWPFTFGPGTKLEIKGGGGSGGGGSGGGGSGGGGSQVQLVQSGAEVKKPGSSMRVSCKASEDTFSSYAISWVRQAPGQGLEWMGGTAPIFGTTNYAQKFQGRVTITADKSTSTAYMELSSLRSEDTAVYYCARGGPYFDYWGQGTLVTVSSGGGASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 362 aa, synthetic construct
  REGION 1..362: note = Alpaca Hinge and human monoFc67
EPKTPKPQPTSGHRGQGGGGSEVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSSASAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 204 - DNA, 1290 bp, synthetic construct
  source 1..1290: mol_type = other DNA
atggagaccgacaccctgctgctgtgggtgctgctgctgtgggtgcccggatccaccggcgaggtgcagctggtggagtctgggggaggcttggtcaagcctggaggatccctgagactctcctgtgcagcctctggattcaccttcagtgacttctacatgagttggatccgccaggctccagggaaggggctggagtggatttcatacattagtagtagtggtagcacccgctactacgcagactctgtgaagggccgattcaccatctccagggacaacgccaagaactcactgtatctgcaaatgaacagcctgagagccgaggacacggccatatattattgtgcgagagatatcccatcgtctagcagcgactgggttgatctacagtttgactactggggccagggaaccctggtcaccgtctcctcaggcggaggaggttcgggaggcggtggtagtggtggaggaggctctgaggtgcagctggtggagtctgggggaggcttggtcaagcctggaggatccctgagactctcctgtgcagcctctggattcaccttcagtgacttctacatgagttggatccgccaggctccagggaaggggctggagtggatttcatacattagtagtagtggtagcacccgctactacgcagactctgtgaagggccgattcaccatctccagggacaacgccaagaactcactgtatctgcaaatgaacagcctgagagccgaggacacggccatatattattgtgcgagagatatcccatcgtctagcagcgactgggttgatctacagtttgactactggggccagggaaccctggtcaccgtctcctcaggaggcggtggtagtggtggaggaggctctggtggcggagggtctcgaatacgtctggtagagtctgggggaggattggtgcaggctgggggctctctgagactctcctgtgcagcctctggacgcacctccggtattcctgccatggcctggttccgccaggctccagggaaggagcgtgagtttgtagctatggcatggagtggtggtggcacaatctatggagactccgtgaaaaaccgattcaccatctccagagacaacacccagaacacgatatttctgcaaatgaacagcctgaaacctgaggacacggccctttattactgtgcagcaggtcggagcactagtaatagggccgaccagtataactactggggccaggggacccaggtcaccgtctcctcagctagccatcatcaccatcaccattga

SEQ ID NO: 205 - DNA, 1653 bp, synthetic construct
  source 1..1653: mol_type = other DNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

(19) United States
(12) Patent Application Publication
(10) Pub. No.: US 2026/0125468 A1
(43) Pub. Date: May 7, 2026

(54) CD3-TARGETING ANTIBODY AND USE THEREOF

(71) Applicant: NONA BIOSCIENCES (SUZHOU) CO., LTD., Suzhou, Jiangsu (CN)

(72) Inventors: Yuexiang YIN, Suzhou, Jiangsu (CN); Gezi JIA, Suzhou, Jiangsu (CN); Jian WANG, Suzhou, Jiangsu (CN); Chengen WANG, Suzhou, Jiangsu (CN); Qiuming PAN, Suzhou, Jiangsu (CN); Yuetao WU, Suzhou, Jiangsu (CN)

(21) Appl. No.: 19/114,651

(22) PCT Filed: Sep. 18, 2023
(86) PCT No.: PCT/CN2023/119524

§ 371 Date: Mar. 24, 2025

(30) Foreign Application Priority Data
Sep. 30, 2022 (WO) PCT/CN2022/123482

(51) Int. Cl. C07K 16/28; A61K 39/00; A61P 35/00

(52) U.S. Cl. CPC C07K 16/2809; A61P 35/00; C07K 16/2803; C07K 16/2827; A61K 2039/505; C07K 2317/22; C07K 2317/24; C07K 2317/31; C07K 2317/33; C07K 2317/569; C07K 2317/73; C07K 2317/75; C07K 2317/92

(57) ABSTRACT

Disclosed herein are CD3-targeting antibodies and bispecific antibodies targeting both CD3 and tumor-associated antigens. Polynucleotides encoding the antibodies, pharmaceutical compositions comprising the antibodies, and methods of producing the antibodies described above are also provided herein. Medical uses of the antibodies described herein are also disclosed.

### TECHNICAL FIELD

The present disclosure relates to molecular biology and immunology. Provided herein include anti-CD3 antibodies comprising a VHH fragment (single variable domain VH of heavy chain antibodies), bispecific antibodies comprising the same, as well as uses thereof in treating diseases.

### BACKGROUND

CD3 targeted bispecific antibodies are an emerging treatment modality in the field of cancer immunotherapy. In 2014, FDA approved the anti-CD3xCD19 bispecific T-cell engager blinatumomab for the treatment of patients with Philadelphia chromosome-negative B cell acute lymphoblastic leukemia. More than 100 clinical trials have been initiated for CD3 bispecific T cell redirection in cancer, and some of them show promising outcome in both hematological and solid tumor.

CD3 bispecific antibodies can simultaneously target CD3 on T cells and tumor-associated antigens (TAA) expressed on cancer cells. Crosslinking of these two cell types by CD3 bispecific antibodies allows the formation of an immunological synapse similar to that of a natural TCR-MHC complex. This synapse results in T cell activation and thereby secreting inflammatory cytokines and cytolytic molecules which are able to kill the cancer cells in the process. Unlike the TCR, CD3 bispecific antibodies are capable of inducing redirected lysis of cancer cells by polyclonal T cells in an MHC-peptide complex independent manner. Therefore, CD3 bispecific antibodies can engage all available T cells, not restricted to tumor specific T cells, to target and to lyse the tumor by the redirection of the other anti-TAA arm.

As new insights in cancer biology accumulate and the antibody technology advances, many different formats of CD3 bispecific have been generated. However, so far, there is still no bispecific utilizing anti-CD3 single domain antibodies reported. VHH antibodies, derived from heavy chain antibodies in llama or alpaca, retain their binding activity in a monomeric manner. This brings the merits for multi-specific antibody construction: first, using VHH instead of VH and VL can reduce the overall chain numbers and obviates the mismatch of unpaired chains; second, due to the strictly monomeric nature, the VHH can be easily grafted onto various parts of multivalent antibodies without compromising qualified biophysical and biochemical properties. As more and more evidences indicate that only first signal or second signal with TAA redirection by bispecific antibodies are far from sufficient to treat solid tumor, there is great demand for trispecific antibodies including one arm targeting CD3 for T cell activation and engagement of tumor lysing, one arm targeting second signal for immune cell proliferation and anti-apoptosis, and the third arm for tumor targeting. Under such circumstances, anti-CD3 VHH antibodies that could be conveniently grafted onto existent costimulatory or immune check point inhibitory bispecific antibodies are of invaluable importance and extreme urgency.

### SUMMARY

Provided herein are antibodies that specifically bind to CD3, bispecific antibodies that further bind to tumor-associated antigens, related pharmaceutical compositions, polynucleotides, vectors, and recombinant cells. Methods of producing the antibodies and uses thereof are also provided herein.

Here we demonstrate the discovery of anti-CD3 VHH antibodies. These anti-CD3 VHHs can bind to human CD3 proteins, can specifically bind to human T lymphocyte cell line Jurkat while not to CD3/TCR knockout Jurkat variant cell line, and can activate Jurkat-NFAT-luciferase cells. Most importantly, when constructed into tumor targeting bispecific antibody with anti-BCMA or anti-B7H4 or anti-ROR1 antibodies, the anti-CD3 VHH can engage T cells for tumor cell lysing. This functional anti-CD3 VHH may play indispensable role in future T cell engaging cancer therapy.

The first aspect of the present disclosure provides a CD3-targeting antibody comprising a heavy chain variable region comprising HCDR1, HCDR2 and HCDR3, wherein the HCDR1 comprises an amino acid sequence as set forth in any one of SEQ ID NOs: 17-18 and 20-22, the HCDR2 comprises an amino acid sequence as set forth in any one of SEQ ID NOs: 41-43, and the HCDR3 comprises an amino acid sequence as set forth in any one of SEQ ID NOs: 62-79.

In some embodiments, the HCDR1, HCDR2 and HCDR3 comprise the amino acid sequence set forth in SEQ ID NOs: 17, 41 and 62, respectively.

In some other embodiments, the HCDR1, HCDR2 and HCDR3 comprise the amino acid sequence set forth in SEQ ID NOs: 18, 41 and 63, respectively.

In some other embodiments, the HCDR1, HCDR2 and HCDR3 comprise the amino acid sequence set forth in SEQ ID NOs: 20, 42 and 64, respectively.

In some other embodiments, the HCDR1, HCDR2 and HCDR3 comprise the amino acid sequence set forth in SEQ ID NOs: 17, 41 and 65, respectively.

In some other embodiments, the HCDR1, HCDR2 and HCDR3 comprise the amino acid sequence set forth in SEQ ID NOs: 20, 42 and 66, respectively.

In some other embodiments, the HCDR1, HCDR2 and HCDR3 comprise the amino acid sequence set forth in SEQ ID NOs: 21, 42 and 64, respectively.

In some other embodiments, the HCDR1, HCDR2 and HCDR3 comprise the amino acid sequence set forth in SEQ ID NOs: 20, 42 and 67, respectively.

In some other embodiments, the HCDR1, HCDR2 and HCDR3 comprise the amino acid sequence set forth in SEQ ID NOs: 20, 42 and 68, respectively.

In some other embodiments, the HCDR1, HCDR2 and HCDR3 comprise the amino acid sequence set forth in SEQ ID NOs: 20, 42 and 69, respectively.

In some other embodiments, the HCDR1, HCDR2 and HCDR3 comprise the amino acid sequence set forth in SEQ ID NOs: 20, 42 and 70, respectively.

In some other embodiments, the HCDR1, HCDR2 and HCDR3 comprise the amino acid sequence set forth in SEQ ID NOs: 20, 42 and 71, respectively.

In some other embodiments, the HCDR1, HCDR2 and HCDR3 comprise the amino acid sequence set forth in SEQ ID NOs: 20, 42 and 72, respectively.

In some other embodiments, the HCDR1, HCDR2 and HCDR3 comprise the amino acid sequence set forth in SEQ ID NOs: 20, 42 and 73, respectively.

In some other embodiments, the HCDR1, HCDR2 and HCDR3 comprise the amino acid sequence set forth in SEQ ID NOs: 20, 42 and 74, respectively.

In some other embodiments, the HCDR1, HCDR2 and HCDR3 comprise the amino acid sequence set forth in SEQ ID NOs: 20, 42 and 75, respectively.

In some other embodiments, the HCDR1, HCDR2 and HCDR3 comprise the amino acid sequence set forth in SEQ ID NOs: 20, 42 and 76, respectively.

In some other embodiments, the HCDR1, HCDR2 and HCDR3 comprise the amino acid sequence set forth in SEQ ID NOs: 20, 42 and 77, respectively.

In some other embodiments, the HCDR1, HCDR2 and HCDR3 comprise the amino acid sequence set forth in SEQ ID NOs: 22, 42 and 64, respectively.

In some other embodiments, the HCDR1, HCDR2 and HCDR3 comprise the amino acid sequence set forth in SEQ ID NOs: 20, 43 and 64, respectively.

In some other embodiments, the HCDR1, HCDR2 and HCDR3 comprise the amino acid sequence set forth in SEQ ID NOs: 20, 42 and 78, respectively.

In some other embodiments, the HCDR1, HCDR2 and HCDR3 comprise the amino acid sequence set forth in SEQ ID NOs: 20, 42 and 79, respectively.

In the present disclosure, the CDR regions are defined as Chothia numbering system.

In some embodiments, the heavy chain variable region (VH) comprises the amino acid sequence as set forth in any one of SEQ ID NOs: 107-108 and SEQ ID NOs: 110-132.

In some other embodiments, the VH fragment has at least 90%, at least 95%, at least 98%, at least 99% of sequence identity to the amino acid sequence as set forth in any one of SEQ ID NOs: 107-108 and SEQ ID NOs: 110-132.

In some embodiments, the antibody further comprises a heavy chain constant region.

In some preferred embodiments, the heavy chain constant region is derived from hIgG1, hIgG2, hIgG3 and hIgG4 and a variant thereof; more preferably, the heavy chain constant region is derived from hIgG1.

In some embodiments, the antibody further comprises one or more mutations at heavy chain constant region.

In some preferred embodiments, the mutation is selected from L234A, L235A, G237A, P329G (Eu numbering) or any combination.

In some embodiments, the antibody is a heavy chain only antibody comprising an amino acid sequence as set forth in any one of SEQ ID NOs: 141-142 and SEQ ID NOs: 144-166.

In some embodiments, the CD3-targeting antibody could be in the format of HCAb, VHH, nanobody, Fab, Fab′, F(ab′)₂, Fd, Fd′, and dAb.

The second aspect of the present disclosure provides a bispecific antibody comprising a first antigen-binding fragment targeting CD3 and a second antigen-binding fragment targeting tumor associated antigen (TAA); wherein the first antigen-binding fragment targeting CD3 is as described in the first aspect.

In some embodiments, the TAA could be any one of CD19, BCMA, TSHR, CD171, CS-1, CLL-1, GD3, Tn Ag, FLT3, CD38, CD123, CD44v6, B7H3, B7H4, KIT, IL-13Ra2, IL-11Ra, PSCA, PSMA, PRSS21, VEGFR2, LewisY, CD24, PDGFR-beta, SSEA-4, MUC1, EGFR, NCAM, CAIX, LMP2, EphA2, sLe, GM3, TGS5, HMWMAA, GD2, FOLR1, FOLR2, TEM1/CD248, TEM7R, CLDN6, CLDN18.2, GPRC5D, CXORF61, CD97, CD179a, ALK, PLAC1, GloboH, NY-BR-1, UPK2, HAVCR1, ADRB3, PANX3, GPR20, LY6K, OR51E2, TAARP, WT1, ETV6-AML, SPA17, XAGE1, Tie 2, MAD-CT-1, MAD-CT-2, FOSL1, hTERT, ML-IAP, ERG, NA17, PAX3, AR, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLECi2A, BST2, EMR2, LY75, GPC3, FCRL5, IGLL1, CD20, CD30, HER2, ROR1, FLT3, TAAG72, CD22, CD33, GD2, gp100Tn, FAP, TYR, EPCAM, CEA, IGF-1R, EphB2, MSLN, CDH17, CD32b, EGFRvIII, GPNMB, GPR64, HER3, LRP6, LYPD8, NKG2D, SLC34A2, SLC39A6, SLITRK6, GUCY2C and TACSTD2.

In some preferred embodiments, the TAA is BCMA, B7H4 or ROR1.

In some specific embodiments, the second antigen-binding fragment targets B7H4.

In some preferred embodiments, the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; the CDR regions are defined as Chothia numbering system.

In some specific embodiments, the second antigen-binding fragment targets BCMA.

In some embodiments, the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 12, 36 and 57, respectively; the CDR regions are defined as Chothia numbering system.

In some other specific embodiments, the second antigen-binding fragment targets ROR1.

In some embodiments, the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 15, 39 and 60, respectively; the CDR regions are defined as Chothia numbering system.

In some exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 62, respectively; the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 12, 36 and 57, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 18, 41 and 63, respectively; the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 12, 36 and 57, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 62, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 18, 41 and 63, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 64, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 65, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 66, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 21, 42 and 64, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 67, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 68, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 69, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 70, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 71, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 72, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 73, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 74, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 75, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 76, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 77, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 22, 42 and 64, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 43 and 64, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 78, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 79, respectively; the second antigen-binding fragment comprises one light chain variable region (VL) and one heavy chain variable region (VH), the VLregion comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively.

In some other exemplary embodiments, the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 62, respectively; the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 15, 39 and 60, respectively.

In the present disclosure, the CDRs are determined by any numbering system known in the art, e.g. Kabat numbering system, Chothia numbering system, AbM numbering system, IMGT numbering system, AHO numbering system, ANARCI numbering system and CONTACT numbering system, etc. In some preferred embodiments, the CDRs are determined by Chothia numbering system.

In the embodiments above, the CDR regions are defined as Chothia numbering system.

In some exemplary embodiments, the first antigen-binding fragment comprises one heavy chain variable region (VH), the VH region comprises the amino acid sequence set forth in SEQ ID NO: 107, the second antigen-binding fragment comprises one heavy chain variable region (VH), the VH region comprises the amino acid sequence set forth in SEQ ID NO: 101.

In some other exemplary embodiments, the first antigen-binding fragment comprises one heavy chain variable region (VH), the VH region comprises the amino acid sequence set forth in SEQ ID NO: 108, the second antigen-binding fragment comprises one heavy chain variable region (VH), the VH region comprises the amino acid sequence set forth in SEQ ID NO: 101.

In some other exemplary embodiments, the first antigen-binding fragment comprises one heavy chain variable region (VH), the VH region comprises the amino acid sequence set forth in SEQ ID NO: 107, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 108, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 110, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 111, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 112, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 113, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 114, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 115, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 116, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 117, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 118, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 119, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 120, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 121, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 122, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 123, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 124, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 125, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 126, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 127, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 128, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 129, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 130, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 131, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 132, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103.

In some other exemplary embodiments, the first antigen-binding fragment comprises one heavy chain variable region (VH), the VH region comprises the amino acid sequence set forth in SEQ ID NO: 107, the second antigen-binding fragment comprises one heavy chain variable region (VH), the VH region comprises the amino acid sequence set forth in SEQ ID NO: 105.

In some embodiments, the bispecific antibody comprises one polypeptide with the amino acid sequence set forth in SEQ ID NO: 172.

In some other embodiments, the bispecific antibody comprises one polypeptide with the amino acid sequence set forth in SEQ ID NO: 173.

In some other embodiments, the bispecific antibody comprises two polypeptides, wherein, the first polypeptide comprises the amino acid sequence set forth in SEQ ID NO: 175, the second polypeptide comprises the amino acid sequence set forth in SEQ ID NO: 170.

In some other embodiments, the bispecific antibody comprises two polypeptides, wherein, the first polypeptide comprises the amino acid sequence set forth in SEQ ID NO: 176, the second polypeptide comprises the amino acid sequence set forth in SEQ ID NO: 170.

In some other embodiments, the bispecific antibody comprises two polypeptides, wherein, the first polypeptide comprises the amino acid sequence set forth in SEQ ID NO: 177, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 179, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 180, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 181, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 182, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 183, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 185, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 186, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 187, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 188, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 189, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 190, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 191, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 192, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 193, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 194, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 195, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 196, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 197, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 198, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 199, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 200, the second polypeptide comprises the amino acid sequence set forth in SEQ ID NO: 178.

In some other embodiments, the bispecific antibody comprises two polypeptides, wherein, the first polypeptide comprises the amino acid sequence set forth in SEQ ID NO: 201, the second polypeptide comprises the amino acid sequence set forth in SEQ ID NO: 178.

In some other embodiments, the bispecific antibody comprises comprising one polypeptide with the amino acid sequence set forth in SEQ ID NO: 184.

In some embodiments, the CD3-targeting antibody is a chimeric antibody, a humanized antibody, or a human antibody.

Herein in the present disclosure, the antibody or antigen-binding fragment binding specially to BCMA, or the antibody or antigen-binding fragment binding specially to B7H4, or the antibody or antigen-binding fragment binding specially to ROR1, is preferably selected from the group consisting of Fab, Fv, VHH and scFv.

Herein in the present disclosure, the antibody or antigen-binding fragment binding specially to BCMA is preferably anti-BCMA VHH; the antibody or antigen-binding fragment binding specially to B7H4 is anti-B7H4 scFv; the antibody or antigen-binding fragment binding specially to ROR1 is preferably anti-ROR1 VHH.

In some embodiments, the bispecific antibody comprises:
a) a polypeptide comprising anti-CD3 VHH-anti-BCMA VHH-mono Fc; or,
b) a first polypeptide comprising anti-CD3 VHH-Fc and a second polypeptide comprising anti-B7H4 scFv-Fc; wherein two Fc portions form a dimeric Fc region (in a format of VH-Fc-scFv); or,
c) a polypeptide comprising two copies of anti-ROR1 VHH and one copy of anti-CD3 VHH.

In some embodiments, one Fc interacts with the other Fc via conventional technical means in the art, such as a chemical bond or a structure of knob(s)-into-hole(s).

In some specific embodiments, the bispecific antibody comprises heavy chain constant regions which could have a combination of mutations comprising L234A and L235A (LALA), or a combination of mutations comprising L234A, L235A and G237A (AAA), or a combination of mutations comprising L234A, L235A and P329G (AAG), to eliminate ADCC effect.

The third aspect of the present disclosure provides a polynucleotide encoding the CD3-targeting antibody described in the first aspect or the bispecific antibody described in the second aspect.

In some embodiments, the polynucleotide is selected from DNA or RNA.

In some embodiments, the polynucleotide is mRNA.

The fourth aspect of the present disclosure provides a vector comprising the polynucleotide described in the third aspect.

The fifth aspect of the present disclosure provides a cell comprising the CD3-targeting antibody as described in the first aspect, the bispecific antibody as described in the second aspect, the polynucleotide as described in the third aspect, or the vector described in the fourth aspect.

The sixth aspect of the present disclosure provides a method for preparing the CD3-targeting antibody as described in the first aspect, the bispecific antibody as described in the second aspect, wherein the method comprises the step of cultivating the cell described in the fifth aspect comprising the polynucleotide or the vector to obtain the CD3-targeting antibody or the bispecific antibody from culture.

The seventh aspect of the present disclosure provides a pharmaceutical composition comprising the CD3-targeting antibody described in the first aspect, or the bispecific antibody described in the second aspect, or the polynucleotide described in the third aspect, or the vector described in the fourth aspect, or the cell described in the fifth aspect, and optionally a pharmaceutically acceptable carrier.

The eighth aspect of the present disclosure provides a use of the CD3-targeting antibody described in the first aspect, or the bispecific antibody described in the second aspect, or the polynucleotide described in the third aspect, or the vector described in the fourth aspect, or the cell described in the fifth aspect, or the pharmaceutical composition described in the seventh aspect for the preparation of a medicament for treating disease.

The ninth aspect of the present disclosure provides the CD3-targeting antibody described in the first aspect, or the bispecific antibody described in the second aspect, or the polynucleotide described in the third aspect, or the vector described in the fourth aspect, or the cell described in the fifth aspect, or the pharmaceutical composition described in the seventh aspect for the use as a medicament; preferably the medicament is used for preventing and/or treating diseases.

The tenth aspect of the present disclosure provides a method for preventing and/or treating a disease in a subject in need thereof, comprising administering an effective amount of the CD3-targeting antibody described in the first aspect, or the bispecific antibody described in the second aspect, or the polynucleotide described in the third aspect, or the vector described in the fourth aspect, or the cell described in the fifth aspect, or the pharmaceutical composition described in the seventh aspect. In some preferred embodiments, the effective amount is therapeutically effective.

The eleventh aspect of the present disclosure provides a composition for use in preventing and/or treating a disease, wherein the composition comprises the CD3-targeting antibody described in the first aspect, or the bispecific antibody described in the second aspect, or the polynucleotide described in the third aspect, or the vector described in the fourth aspect, or the cell described in the fifth aspect, or the pharmaceutical composition described in the seventh aspect.

The twelfth aspect of the present disclosure provides a composition for use in preparing a medicament for preventing and/or treating a disease, wherein the composition comprises the CD3-targeting antibody described in the first aspect, or the bispecific antibody described in the second aspect, or the polynucleotide described in the third aspect, or the vector described in the fourth aspect, or the cell described in the fifth aspect, or the pharmaceutical composition described in the seventh aspect.

Herein in the present disclosure, the disease is selected from the group consisting of myeloma, prostate cancer, liver cancer, kidney cancer, pancreatic cancer, breast cancer, cervical cancer, ovarian cancer, colorectal cancer, lung cancer, breast cancer, nasopharyngeal cancer, oral cancer, esophageal cancer, stomach cancer, bladder cancer and lymphoma.

### BRIEF DESCRIPTION OF THE DRAWINGS

FIG. 1 illustrates the results of binding activity of antibodies to Jurkat or J.RT3-T3.5 cells determined by FACS.

FIG. 2 illustrates the results of binding activity of antibodies to CHOK1/TCR/CD3 or CHO-K1 cells determined by FACS.

FIG. 3 illustrates the luciferase luminescence signals raised by Jurkat-NFAT-Luc reporter cells upon activation by engagement of anti-CD3 antibodies.

FIG. 4 illustrates the structures of fusing VHH regions to monoFc or conventional human IgG1 Fc to generate recombinant antibodies of various formats.

FIG. 5 illustrates the SDS-PAGE results of recombinant A1-monoFc (PR005860) and E2-monoFc (PR005861) after transient expression and affinity capture.

FIG. 6 illustrates the T cell-mediated cytotoxicity of A1-monoFc (PR005860) to BCMA highly expressed NCI-H929 cells is dose dependent.

FIG. 7 illustrates the HCAb format of PR006364 (A1-HCAb) and PR010991 (3G11-HCAb).

FIG. 8 illustrates the results of binding activity of antibodies to Jurkat (A) or J.RT3-T3.5 (B) or Jurkat-NFAT-Luc cells (C) determined by FACS.

FIG. 9 illustrates the results of binding to various CD3 recombinant proteins by ELISA (A-F).

FIG. 10 illustrates the bispecific antibodies formats of VH-Fc-scFv and Fab-Fc-scFv; LALA mutation refers to a combination of mutations comprising L234A and L235A.

FIG. 11 illustrates the cytotoxicity of B7H4xCD3 bispecific antibodies to B7H4 highly expressed MDA-MB-468 cells determined by RTCA method (A-B).

FIG. 12 illustrates the binding activities to human CD3e&d heterodimer protein for anti-CD3 A1-derived humanized variants.

FIG. 13 illustrates the binding activities to Jurkat-NFAT-Luc reporter cells for anti-CD3 A1-derived humanized variants.

FIG. 14 illustrates the cytotoxicity to B7H4 highly expressed MDA-MB-468 cells for anti-CD3 humanized A1 variants derived B7H4xCD3 bispecific antibodies determined by RTCA method.

FIG. 15 illustrates the cytotoxicity to B7H4-negative MDA-MB-231 cells for anti-CD3 humanized A1 variants derived B7H4xCD3 bispecific antibodies determined by RTCA method.

FIG. 16 illustrates the binding activities to cynomolgus monkey PBMCs determined by FACS (A-B).

FIG. 17 illustrates the cytotoxicity to MDA-MB-468 cells in the presence of cynomolgus monkey PBMCs for anti-CD3 3G11 derived B7H4xCD3 bispecific antibody PR008551 (A-B).

FIG. 18 illustrates the alignment for VH sequences of 3G11 VH and its variants.

FIG. 19 illustrates the cytotoxicity to MDA-MB-468 cells for B7H4xCD3 bispecific antibodies generated from 3G11-derived variants (A-C).

FIG. 20 illustrates the cytotoxicity to PANC-1 cells for anti-CD3 A1 derived ROR1xCD3 bispecific antibody.

FIG. 21 illustrates the tumor volume changes for each treatment group in PANC-1 PBMC model.

### DETAILED DESCRIPTION

### Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

It is to be noted that the term “a” or “an” entity refers to one or more of that entity; for example, “an antibody” is understood to represent one or more antibodies. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “polypeptide”, “peptide”, “protein”, “polypeptide chain”, “peptide chain”, and their grammatical equivalents as used interchangeably herein refer to polymers of amino acids of any length, which can be linear or branched. It can include unnatural or modified amino acids or be interrupted by non-amino acids. A polypeptide, peptide, polypeptide chain, peptide chain, or protein can also be modified with, for example, disulfide bond formation, glycosylation, lipidation, acetylation, phosphorylation, or any other manipulation or modification.

The terms “polynucleotide”, “nucleic acid”, and their grammatical equivalents as used interchangeably herein mean polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase.

The term “specifically binds”, as used herein, means that a polypeptide or molecule interacts more frequently, more rapidly, with greater duration, with greater affinity, or with some combination of the above to the epitope, protein, or target molecule than with alternative substances, including related and unrelated proteins.

The terms “identical”, percent “identity”, and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity.

The terms “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity for binding to the antigen. Examples of such include, but are not limited to a complementarity determining region (CDR) of a heavy or light chain or a ligand binding portion thereof, a heavy chain or light chain variable region, a heavy chain or light chain constant region, a framework (FWR) region, or any portion thereof, or at least one portion of a binding protein. The CDRs of the light and heavy chains are primarily responsible for the interaction of the antibody with the antigen. Each VH or VL is typically composed of three CDRs and four FRs, which are arranged from amino-terminus to carboxy-terminus in the following order: FWR1, CDR1, FWR2, CDR2, FWR3, CDR3, and FWR4 (see also Chothia and Lesk, J Mol Biol 1987; 196:901-17).

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR regions (HCDR1, HCDR2, HCDR3 refer to 3 CDRs of VH; LCDR1, LCDR2, LCDR3 refer to 3 CDRs of VL) are well known to those skilled in the art and have been defined by a variety of methods/systems. These systems and/or definitions have been developed and refined over years and include Kabat, Chothia, IMGT, AbM, and CONTACT. Software programs (e.g., abYsis) are available and known to those of skill in the art for analysis of antibody sequence and determination of CDRs. Unless otherwise specified, the amino acid sequences are described with Chothia numbering system.

The term “antibody” encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, etc., are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. The structure of immunoglobulins has been well characterized (see, e.g., Fundamental Immunology Ch. 7 (Paul, W., ed., 2nd ed. Raven Press, N.Y. (1989)). Typically, immunoglobulins comprise two pairs of polypeptide chains, one pair of light chains and one pair of heavy chains, all four inter-connected by disulfide bonds.

Each light chain of an immunoglobulin typically includes a light chain variable region (“VL region”) and a light chain constant region (“CL region”). There are two distinct types of light chains, referred to as kappa (κ) of lambda (λ) based on the amino acid sequence of the CL region. The amino acid sequences of the CL regions are well known in the art. Each heavy chain typically includes a heavy chain variable region (a “VH region”) and a heavy chain constant region (a “CH region”).

A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art and are described.

The term “Fc region” as used herein includes native sequence Fc regions and variant Fc regions. In some embodiments, the Fc domains of the two heavy chains of a bispecific antibody provided herein can comprise paired modifications that promote their association with each other, instead of forming homodimers.

The terms “antibody fragment” or “antigen-binding fragment”, as used herein, is a portion of an antibody such as F(ab′)₂, F(ab)₂, Fab′, Fab, Fv, single chain fragment variable (scFv) and the like. Regardless of structure, an antibody fragment binds with the same antigen that is recognized by the intact antibody. The term “antibody fragment” includes aptamers, spiegelmers, Fab, Fab′, F(ab′)₂, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments. The term “antibody fragment” also includes any synthetic or genetically engineered protein that acts like an antibody by binding to a specific antigen to form a complex.

The term “humanized”, “humanization” or “human-like” are used interchangeably to refer to an antibody comprising in at least one of its binding domains at least one complementarity determining region (“CDR”) from a non-human antibody or fragment thereof. Humanization approaches are described for example in WO 91/09968 and U.S. Pat. No. 6,407,213. As non-limiting examples, the term encompasses the case in which a variable region of at least one binding domain comprises a single CDR region, for example the third CDR region of the VH, from another non-human animal, for example a rodent, as well as the case in which a or both variable region/s comprise at each of their respective first, second and third CDRs the CDRs from said non-human animal. In the event that all CDRs of a binding domain of the antibody have been replaced by their corresponding equivalents from, for example, a rodent, one typically speaks of “CDR-grafting”, and this term is to be understood as being encompassed by the term “humanized” or grammatically related variants thereof as used herein. The term “humanized” or grammatically related variants thereof also encompasses cases in which, in addition to replacement of one or more CDR regions within a VH and/or VL of the first and/or second binding domain further mutation/s (e.g. substitutions) of at least one single amino acid residue/s within the framework (“FR”) regions between the CDRs has/have been effected such that the amino acids at that/those positions correspond/s to the amino acid/s at that/those position/s in the animal from which the CDR regions used for replacement is/are derived. As is known in the art, such individual mutations are often made in the framework regions following CDR-grafting in order to restore the original binding affinity of the non-human antibody used as a CDR-donor for its target molecule. The term “humanized” may further encompass (an) amino acid substitution(s) in the CDR regions from a non-human animal to the amino acid(s) of a corresponding CDR region from a human antibody, in addition to the amino acid substitutions in the framework regions as described above.

### Polynucleotides

The term “polynucleotide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA can be cDNA, genomic DNA, or synthetic DNA, and can be double-stranded or single-stranded. Single stranded DNA can be the coding (sense) strand or non-coding (anti-sense) strand. The polynucleotides of the disclosure can be mRNA.

In some embodiments, provided herein are polynucleotides encoding the anti-CD3 antibodies and bispecific antibodies described above. As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide is isolated. In some embodiments, a polynucleotide is substantially pure.

### Vectors

The term “vector” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product, and it is further understood that expression levels can be optimized to obtain sufficient expression using methods well known in the art.

### Cells

The host cell can be co-transfected with more than one expression vectors, each encoding a polypeptide chain of the antibody described herein. The vectors can contain identical selectable markers which enable equal expression of all polypeptides. Alternatively, a single vector can be used which encodes two or more polypeptides. The coding sequences for the polypeptides of compounds described herein can comprise cDNA or genomic DNA.

### Methods for Production

The antibodies or polypeptides described herein can be produced and isolated using methods known in the art. Polypeptides can be synthesized, in whole or in part, using chemical methods. Peptide synthesis can be performed using various solid phase techniques and automated synthesis may be achieved. Peptides can also be synthesized using combinatorial methodologies. Synthetic residues and polypeptides can be synthesized using a variety of procedures and methodologies known in the art.

A variety of host-expression vector systems can be utilized to recombinantly express the antibodies described herein or one or more of their polypeptide chains. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art. Such host-expression systems represent vehicles by which the coding sequences of the antibodies described herein can be produced and subsequently purified, but also represent cells which may, when transformed or transfected with the appropriate polynucleotide coding sequences, express the antibodies described herein in situ.

Once an antibody described herein or polypeptide described herein has been recombinantly expressed, it can be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the compound comprises an Fc domain (or portion thereof)), and sizing column chromatography), centrifugation, differential solubility, or by any other standard technique for the purification of polypeptides or antibodies.

### Pharmaceutical Compositions

The term “pharmaceutically acceptable carrier” or “pharmaceutical excipient” refers to a material that is suitable for drug administration to an individual along with an active agent without causing undesirable biological effects or interacting in a deleterious manner with any of the other components of the pharmaceutical composition.

In some embodiments, the pharmaceutical compositions disclosed herein can comprise one or more of a buffer system, a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. The formulation should suit the mode of administration. The parental preparation can be enclosed in ampoules, disposable syringes or multiple dose vials made of glass or plastic.

The amount of the antibodies, fragments, or antibody-drug conjugates of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

### Therapeutic Methods

The term “administration” or “administering” and their grammatical equivalents as used herein refer to the act of delivering, or causing to be delivered, a therapeutic or a pharmaceutical composition to the body of a subject by a method described herein or otherwise known in the art. Administration can be systemic or local. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

In some embodiments, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

Terms “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

The examples provided below are for purposes of illustration only, which are not intended to be limiting unless otherwise specified. Thus, the disclosure should in no way be construed as being limited to the following examples, but rather, should be construed to encompass any and all variations which become evident as a result of the teaching provided herein.

Below presents preferred embodiments of the present disclosure based on the drawings in order to illustrate the technical schemes of the present disclosure in detail.

### EXAMPLES

### Example 1: Immunization of Alpacas and Screening of Anti-CD3 VHH Antibodies

Alpacas were immunized with human pan T cells at intervals of 3 weeks. After more than 3 months immunization, 50 mL alpaca blood was extracted for PBMC separation. Phage libraries encoding VHH gene fragments were constructed by standard protocol (DOI:10.1038/nprot.2014.039).

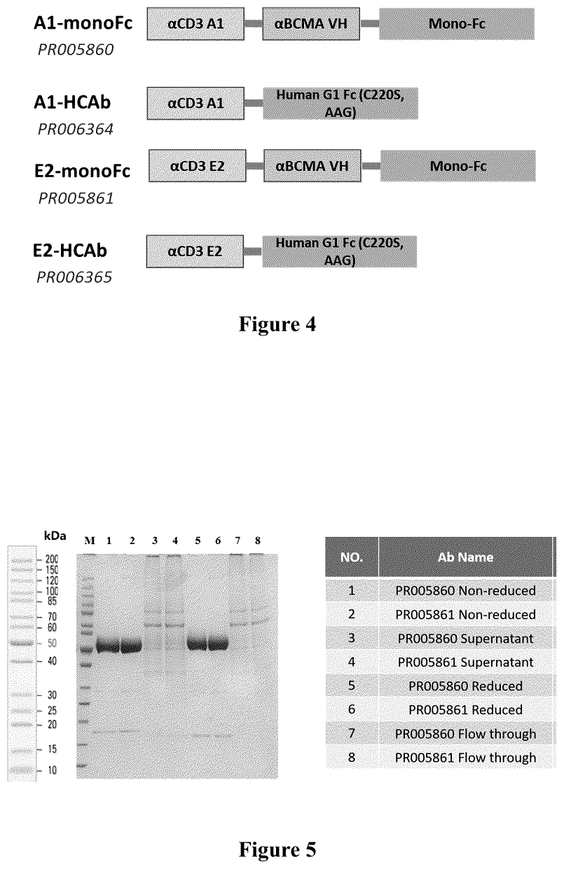

In first panning campaign, phage libraries were panned against cynomolgus CD3 epsilon (cyno CD3e) protein (ACRO Biosystems, CDE-C5226) and Jurkat (ATCC, TIB-152) cells. VHH genes from the panning output pool were cloned into pTT5-VHH-anti-BCMA-his vector which concatenated VHH gene fragments to the N-terminus of VH region (SEQ ID NO: 101) from anti-BCMA HCAb PR001046 identified previously (published in patent WO2021063349A1), and then the individual plasmids were prepared before transfection. HEK293T cells were transfected with each single plasmid carries anti-CD3 VHH and anti-BCMA VH in tandem, and the supernatants were harvested at 96 hours after transfection. The supernatants were then screened for binding to Jurkat cells by FACS. Two clones (A1 and E2) were identified as positive hits of binding to Jurkat, and their VHH genes were cloned into pTT5-VHH-anti-BCMA-monoFc vector for protein expression, denoted as A1-monoFc (PR005860) and E2-monoFc (PR005861) respectively as shown in FIG. 4. The amino acid sequences of A1 and E2 and their CDR regions are listed in Table 1.

In second panning campaign, phage libraries were panned against cynomolgus CD3 epsilon&delta heterodimeric (cyno CD3e/d) protein (ACRO Biosystems, CDD-C52W9) and cynomolgus PBMC cells. VHH genes from the panning output pool were cloned into pTT5-VHH-monoFc vector which carries human IgG Fc variant that can properly express as monomeric form instead of homodimeric form for conventional Fc. HEK293T cells were transfected with each single plasmid prepared, and the supernatants were harvested at 96 hours after transfection. The supernatants were then screened for binding to Jurkat cells by FACS. One clone (3G11) was identified as positive hit of binding to Jurkat, and its VHH gene was cloned into pTT5-VHH-hFc vector for protein expression, denoted as HCAb PR010991 shown in FIG. 7. The amino acid sequences of 3G11 and their CDR regions are listed in Table 1.

TABLE 1
Sequences of Clones A1, E2 and 3G11 (defined by Chothia numbering system)

| Clone | HCDR1 | HCDR2 | HCDR3 | VH |
|---|---|---|---|---|
| A1 | GRTSGIP | WSGGG | GRSTSNRA | RIRLVESGGGLVQAGGSLRLSCAASGRTS |
| | (SEQ ID NO: | (SEQ ID NO: | DQYNY | GIPAMAWFRQAPGKEREFVAMAWSGGG |
| | 17) | 41) | (SEQ ID NO: | TIYGDSVKNRFTISRDNTQNTIFLQMNSL |
| | | | 62) | KPEDTALYYCAAGRSTSNRADQYNYWG |
| | | | | QGTQVTVSS |
| | | | | (SEQ ID NO: 107) |
| E2 | GRTNGIP | WSGGG | GRTTSNKA | EVQLVESGGGLVQAGGSLRLSCAASGRT |
| | (SEQ ID NO: | (SEQ ID NO: | DHYNY | NGIPAMAWFRQAPGKEREFVAMAWSGG |
| | 18) | 41) | (SEQ ID NO: | GTIYGDSVKNRFTISRNNAQNTISLQMSS |
| | | | 63) | LRPEDTALYFCAAGRTTSNKADHYNYW |
| | | | | GQGTQVTVSS |
| | | | | (SEQ ID NO: 108) |
| 3G11 | GSRLNDN | FWTGGN | SRVVWYYC | RVHLAESGGGLVQAGGSLRLSCAASGSR |
| | (SEQ ID NO: | (SEQ ID NO: | SGYGCSRA | LNDNAMAWFRQAPGKEREFVGSIFWTG |
| | 20) | 42) | GDYVY | GNTYYADAVKGRLTISRDNAKNALYLQ |
| | | | (SEQ ID NO: | MNSLKPEDTAVYYCAASRVVWYYCSGY |
| | | | 64) | GCSRAGDYVYWGQGTQVTVSS |
| | | | | (SEQ ID NO: 110) |

As depicted in FIG. 4, A1 and E2 VHH genes were cloned into pTT5-VHH-anti-BCMA-monoFc vector that carries mutated human Fc sequence monoFc67 (SEQ ID NO: 203) to generate recombinant antibodies A1-monoFc (PR005860) and E2-monoFc (PR005861), respectively. PR005860 and PR005861 share same anti-BCMA VH fragment. Herein, the amino acid sequence of the anti-BCMA VH fragment is set forth in SEQ ID NO: 101. The HCDR1, HCDR2 and HCDR3 of this anti-BCMA VH fragment are set forth in SEQ ID NO: 12, 36 and 57, as defined by Chothia numbering system, respectively.

Additionally, A1 and E2 VHH genes were cloned into pTT5-VHH-hFc vector to generate recombinant antibodies A1-HCAb (PR006364) and E2-HCAb (PR006365), respectively. Mutation C220S was introduced to hinge region (between VH and CH2 of Fc) to avoid disulfide bond formation between Cys residues at position 220 (Eu numbering system) or other free thiol group. To abolish the Fc effector functions like ADCC, triple mutations L234A, L235A, P329G (denoted as “AAG”) were introduced to positions 234, 235 and 329 (Eu numbering system) of CH2 domain, respectively.

As depicted in FIG. 7, 3G11 VHH gene was cloned into pTT5-VHH-hFc vector to generate recombinant antibody 3G11-HCAb (PR010991). Mutation C220S was introduced to hinge region; and triple mutations L234A, L235A, G237A (denoted as “AAA”) were introduced to CH2 to remove Fc effector functions.

The amino acid sequences of A1-monoFc (PR005860), E2-monoFc (PR005861), A1-HCAb (PR006364), E2-HCAb (PR006365) and 3G11-HCAb (PR010991) are shown in Table 2.

TABLE 2
Sequences of recombinant antibodies derived from A1, E2, 3G11.

| SEQ ID NO: | PR006364 | anti-CD3 A1-HCAb hIgG1(C220S, AAG) |
|---|---|---|
| 141 | PR006364 Heavy Chain | RIRLVESGGGLVQAGGSLRLSCAASGRTSGIPAMA |
| | Sequence | WFRQAPGKEREFVAMAWSGGGTIYGDSVKNRFTI |
| | | SRDNTQNTIFLQMNSLKPEDTALYYCAAGRSTSNR |
| | | ADQYNYWGQGTQVTVSSASEPKSSDKTHTCPPCP |
| | | APEAAGGPSVFLFPPKPKDTLMISRTPEVTCVVVDV |
| | | SHEDPEVKFNWYVDGVEVHNAKTKPREEQYNSTY |
| | | RVVSVLTVLHQDWLNGKEYKCKVSNKALGAPIEK |
| | | TISKAKGQPREPQVYTLPPSREEMTKNQVSLTCLV |
| | | KGFYPSDIAVEWESNGQPENNYKTTPPVLDSDGSF |
| | | FLYSKLTVDKSRWQQGNVFSCSVMHEALHNHYTQ |
| | | KSLSLSPGK |
| 107 | PR006364 VH Sequence | RIRLVESGGGLVQAGGSLRLSCAASGRTSGIPAMA |
| | | WFRQAPGKEREFVAMAWSGGGTIYGDSVKNRFTI |
| | | SRDNTQNTIFLQMNSLKPEDTALYYCAAGRSTSNR |
| | | ADQYNYWGQGTQVTVSS |
| 7 | PR006364 VH FWR1 | RIRLVESGGGLVQAGGSLRLSCAAS |
| | (Chothia) | |
| 17 | PR006364 VH CDR1 | GRTSGIP |
| | (Chothia) | |
| 29 | PR006364 VH FWR2 | AMAWFRQAPGKEREFVAMA |
| | (Chothia) | |
| 41 | PR006364 VH CDR2 | WSGGG |
| | (Chothia) | |
| 50 | PR006364 VH FWR3 | TIYGDSVKNRFTISRDNTQNTIFLQMNSLKPEDTAL |
| | (Chothia) | YYCAA |
| 62 | PR006364 VH CDR3 | GRSTSNRADQYNY |
| | (Chothia) | |
| 82 | PR006364 VH FWR4 | WGQGTQVTVSS |
| | (Chothia) | |
| SEQ ID NO: | PR006365 | anti-CD3 E2-HCAb hIgG1(C220S, AAG) |
| 142 | PR006365 Heavy Chain | EVQLVESGGGLVQAGGSLRLSCAASGRTNGIPAMA |
| | Sequence | WFRQAPGKEREFVAMAWSGGGTIYGDSVKNRFTI |
| | | SRNNAQNTISLQMSSLRPEDTALYFCAAGRTTSNK |
| | | ADHYNYWGQGTQVTVSSASEPKSSDKTHTCPPCP |
| | | APEAAGGPSVFLFPPKPKDTLMISRTPEVTCVVVDV |
| | | SHEDPEVKFNWYVDGVEVHNAKTKPREEQYNSTY |
| | | RVVSVLTVLHQDWLNGKEYKCKVSNKALGAPIEK |
| | | TISKAKGQPREPQVYTLPPSREEMTKNQVSLTCLV |
| | | KGFYPSDIAVEWESNGQPENNYKTTPPVLDSDGSF |
| | | FLYSKLTVDKSRWQQGNVFSCSVMHEALHNHYTQ |
| | | KSLSLSPGK |
| 108 | PR006365 VH Sequence | EVQLVESGGGLVQAGGSLRLSCAASGRTNGIPAMA |
| | | WFRQAPGKEREFVAMAWSGGGTIYGDSVKNRFTI |
| | | SRNNAQNTISLQMSSLRPEDTALYFCAAGRTTSNK |
| | | ADHYNYWGQGTQVTVSS |
| 8 | PR006365 VH FWR1 | EVQLVESGGGLVQAGGSLRLSCAAS |
| | (Chothia) | |
| 18 | PR006365 VH CDR1 | GRTNGIP |
| | (Chothia) | |
| 29 | PR006365 VH FWR2 | AMAWFRQAPGKEREFVAMA |
| | (Chothia) | |
| 41 | PR006365 VH CDR2 | WSGGG |
| | (Chothia) | |
| 51 | PR006365 VH FWR3 | TIYGDSVKNRFTISRNNAQNTISLQMSSLRPEDTAL |
| | (Chothia) | YFCAA |
| 63 | PR006365 VH CDR3 | GRTTSNKADHYNY |
| | (Chothia) | |
| 82 | PR006365 VH FWR4 | WGQGTQVTVSS |
| | (Chothia) | |
| SEQ ID NO: | PR010991 | anti-CD3 3G11-HCAb hIgG1(C220S, AAA) |
| 144 | PR010991 Heavy Chain | RVHLAESGGGLVQAGGSLRLSCAASGSRLNDNAM |
| | Sequence | AWFRQAPGKEREFVGSIFWTGGNTYYADAVKGRL |
| | | TISRDNAKNALYLQMNSLKPEDTAVYYCAASRVV |
| | | WYYCSGYGCSRAGDYVYWGQGTQVTVSSASEPK |
| | | SSDKTHTCPPCPAPEAAGAPSVFLFPPKPKDTLMIS |
| | | RTPEVTCVVVDVSHEDPEVKFNWYVDGVEVHNA |
| | | KTKPREEQYNSTYRVVSVLTVLHQDWLNGKEYKC |
| | | KVSNKALPAPIEKTISKAKGQPREPQVYTLPPSREE |
| | | MTKNQVSLTCLVKGFYPSDIAVEWESNGQPENNY |
| | | KTTPPVLDSDGSFFLYSKLTVDKSRWQQGNVFSCS |
| | | VMHEALHNHYTQKSLSLSPGK |
| 110 | PR010991 VH Sequence | RVHLAESGGGLVQAGGSLRLSCAASGSRLNDNAM |
| | | AWFRQAPGKEREFVGSIFWTGGNTYYADAVKGRL |
| | | TISRDNAKNALYLQMNSLKPEDTAVYYCAASRVV |
| | | WYYCSGYGCSRAGDYVYWGQGTQVTVSS |
| 10 | PR010991 VH FWR1 | RVHLAESGGGLVQAGGSLRLSCAAS |
| | (Chothia) | |
| 20 | PR010991 VH CDR1 | GSRLNDN |
| | (Chothia) | |
| 31 | PR010991 VH FWR2 | AMAWFRQAPGKEREFVGSI |
| | (Chothia) | |
| 42 | PR010991 VH CDR2 | FWTGGN |
| | (Chothia) | |
| 53 | PR010991 VH FWR3 | TYYADAVKGRLTISRDNAKNALYLQMNSLKPEDT |
| | (Chothia) | AVYYCAA |
| 64 | PR010991 VH CDR3 | SRVVWYYCSGYGCSRAGDYVY |
| | (Chothia) | |
| 82 | PR010991 VH FWR4 | WGQGTQVTVSS |
| | (Chothia) | |
| SEQ ID NO: | PR005860 | anti-BCMAxCD3 A1-monoFc |
| 172 | Chain Sequence (AA) | RIRLVESGGGLVQAGGSLRLSCAASGRTSGIPAMA |
| | | WFRQAPGKEREFVAMAWSGGGTIYGDSVKNRFTI |
| | | SRDNTQNTIFLQMNSLKPEDTALYYCAAGRSTSNR |
| | | ADQYNYWGQGTQVTVSSEPKTPKPQPTSGHRGQG |
| | | GGGSEVQLVETGGGLIQPGGSLRLSCAASGFTVSD |
| | | NYMTWVRQAPGKGLEWVSVIFSGGNTYYADSVK |
| | | GRFTISRDNAKNTLYLQMNSLRAEDTALYYCARR |
| | | NYDDTRGTDVFDIWGQGTMVTVSSASAPELLGGPS |
| | | VFLFPPKPKDTLMISRTPEVTCVVVDVSHEDPEVKF |
| | | NWYVDGVEVHNAKTKPREEQYNSTYRVVSVLTVL |
| | | HQDWLNGKEYKCKVSNKALPAPIEKTISKAKGQPR |
| | | EPQVYTKPPSRDELTKNQVSLSCLVKGFYPSDIAVE |
| | | WESNGQPENNYKTTVPVLDSDGSFRLASYLTVDKS |
| | | RWQQGNVFSCSVMHEALHNHYTQKSLSLSPGK |
| SEQ ID NO: | PR005861 | anti-BCMAxCD3 E2-monoFc |
| 173 | Chain Sequence (AA) | EVQLVESGGGLVQAGGSLRLSCAASGRTNGIPAMA |
| | | WFRQAPGKEREFVAMAWSGGGTIYGDSVKNRFTI |
| | | SRNNAQNTISLQMSSLRPEDTALYFCAAGRTTSNK |
| | | ADHYNYWGQGTQVTVSSEPKTPKPQPTSGHRGQG |
| | | GGGSEVQLVETGGGLIQPGGSLRLSCAASGFTVSD |
| | | NYMTWVRQAPGKGLEWVSVIFSGGNTYYADSVK |
| | | GRFTISRDNAKNTLYLQMNSLRAEDTALYYCARR |
| | | NYDDTRGTDVFDIWGQGTMVTVSSASAPELLGGPS |
| | | VFLFPPKPKDTLMISRTPEVTCVVVDVSHEDPEVKF |
| | | NWYVDGVEVHNAKTKPREEQYNSTYRVVSVLTVL |
| | | HQDWLNGKEYKCKVSNKALPAPIEKTISKAKGQPR |
| | | EPQVYTKPPSRDELTKNQVSLSCLVKGFYPSDIAVE |
| | | WESNGQPENNYKTTVPVLDSDGSFRLASYLTVDKS |
| | | RWQQGNVFSCSVMHEALHNHYTQKSLSLSPGK |

### Example 2: Evaluate Binding Activity to CD3-Expressing Cells for Supernatants of Recombinant Anti-CD3 Antibodies

Plasmids of A1-monoFc (PR005860) and E2-monoFc (PR005861) were transfected into 293F host cells (ThermoFisher, R79007) separately. After cell culture for 3-5 days, supernatants were harvested and then exchanged to PBS buffer by 10 KDa ultracentrifuge. The antibody containing PBS solution was defined as bulk for binding and reporter gene assays.

### Binding Activity to Jurkat or J.RT-T3.5 Cell Line

J.RT3-T3.5 cell (ATCC, TIB-153) is a derivative mutant of the Jurkat leukemia cell line (ATCC, TIB-152) that lacks the beta chain of the T-cell antigen receptor, and does not express either CD3 or T cell receptor alpha/beta heterodimer on the cell surface.

The binding activities to target cells for the supernatants A1-monoFc (PR005860) and E2-monoFc (PR005861) were evaluated by FACS. The supernatants of the recombinant antibodies (i.e. A1-monoFc, E2-monoFc) were diluted from bulk solution to 8 different concentrations by 3-fold serial dilution. The diluted samples were incubated with target cells (Jurkat cells (ATCC, TIB-152) or J.RT-T3.5 cells (ATCC, TIB-153)) for 1 hour, and then the secondary antibody (Alexa Fluor®488 AffinityPure Goat Anti-Human IgG (Fcγ Fragment Specific), Jackson ImmunoResearch, 109-545-098) was added and incubated for 1 hour. The cells were washed twice with staining buffer and subjected to flow cytometry. The data were processed by FlowJo v10 (FlowJo, LLC) and non-linear curve fitting analyzed by GraphPad Prism 8.

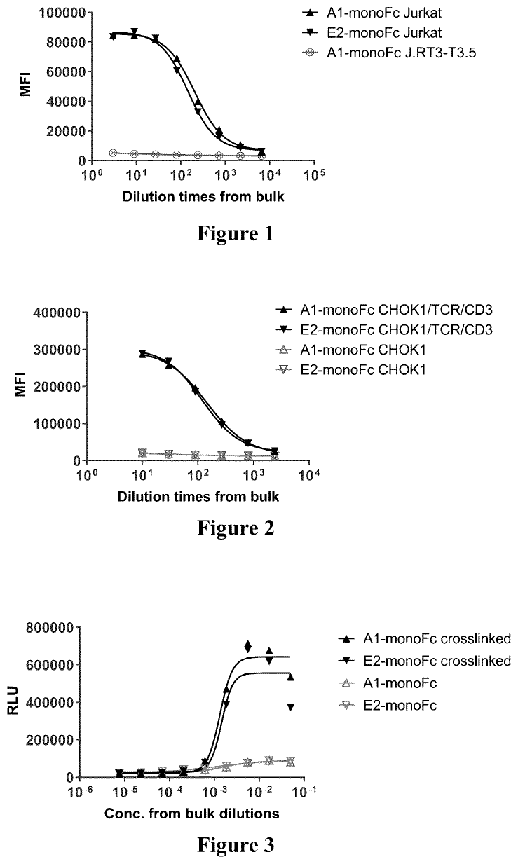

As shown in FIG. 1, the recombinant antibodies A1-monoFc (PR005860) and E2-monoFc (PR005861) can strongly bind to CD3/TCR-positive Jurkat cells in dose-dependent manner with negligible interaction to CD3/TCR-negative J.RT-T3.5 cells, suggesting they could specifically bind to CD3/TCR complex.

### Binding Activity to CHOK1/TCR/CD3 Recombinant Cell Line

CHOK1/TCR/CD3 is a recombinant stable cell line generated on CHO-K1 (ATCC, CCL-61) with over-expression of human CD3/TCR complex with multiple polypeptide subunits including human TCR alpha chain, TCR beta chain, human CD3 zeta chain, CD3 epsilon chain, CD3 delta chain, CD3 gamma chain. The native CHO-K1 cell does not express either CD3 or TCR.

The supernatants of the recombinant antibodies (i.e. A1-monoFc, E2-monoFc) were diluted from bulk solution to 6 different concentrations by 3-fold serial dilution. The diluted samples were incubated with CHO-K1 or CHOK1/TCR/CD3 cells for 1 hour, and then the secondary antibody (Alexa Fluor®488 AffinityPure Goat Anti-Human IgG (Fcγ Fragment Specific), Jackson ImmunoResearch, 109-545-098) was added and incubated for 1 hour. The cells were washed twice with staining buffer and subjected to flow cytometry. The data were processed by FlowJo v10 (FlowJo, LLC) and non-linear curve fitting analyzed by GraphPad Prism 8.

As shown in FIG. 2, A1-monoFc and E2-monoFc can strongly bind to CHOK1/TCR/CD3 cells but can not bind to CHO-K1 cells, suggesting they could specifically bind to CD3/TCR complex.

### Example 3: T Cell Activation Bioassay by Jurkat-NFAT-Luc Reporter Cell

Jurkat-NFAT-Luc is a genetically engineered Jurkat cell line that expresses endogenous TCR, CD3, CD28 receptors and a luciferase reporter driven by an NFAT-response element. When the TCR/CD3 complex on Jurkat-NFAT-Luc is engaged with anti-TCR/CD3 antibody, TCR/CD3 transduces intracellular downstream NFAT transcription, and the expressed NFAT transcription factor can bind to promoter of luciferase gene and regulate the luciferase gene expression. At the presence of substrate, the amount of luciferase can be quantified and reflect the TCR/CD3 activation.

Jurkat-NFAT-Luc reporter cell assay was used to evaluate the T cell activation activities of anti-CD3 antibodies. The cells were seeded at 1×10⁵ cells/well in 50 μL in completed medium. The supernatants of the recombinant antibodies (i.e. A1-monoFc, E2-monoFc) were diluted from bulk solution to 9 different concentrations by 3-fold serial dilution. The diluted samples were added at 25 μL per well, with duplicates. For each diluted sample, two duplicated wells were added with 25 μL crosslinking antibody (Goat F(ab′)2 Anti-Human IgG-Fc (Abcam, ab98587)) with final concentration of 100 nM or added with 25 μL completed medium as non-crosslinking control, respectively. Plates with diluted samples and cells were incubated at 37° C. for 6 hours, and ONE-Glo™ Luciferase Assay System (Promega, E6110) was used for quantified the luminescence signals. The data acquisition and processing were followed with the protocol provided by the vendor (Promega, J1621). Graph data as RLU (relative light unit) versus Log 10 of antibody concentration or bulk dilution, non-linear curve fitting, EC50 determination were analyzed with GraphPad Prism 8.

As shown in FIG. 3, A1-monoFc and E2-monoFc can activate Jurkat-NFAT-Luc reporter cells in dose dependent manner in the presence of crosslinking antibodies. But when there were no crosslinking antibodies in the antibody and cell mixture, A1-monoFc and E2-monoFc can barely activate the reporter cells compared to the crosslinked counterparts. The result suggests that A1-monoFc and E2-monoFc are crosslinking dependent TCR/CD3 agonistic antibodies.

### Example 4: Antibody Production and Purification

This example describes the procedure of generating purified recombinant antibodies listed in Table 2 or other antibodies of this invention.

The plasmid encoding specific recombinant antibody was transiently transfected into 293-F cells (ThermoFisher, R79007) with PEI (Polyscience, 24885). After transfection, the cells were incubated at 37° C. with 5% CO2 and shaking at 120 rpm. The cell culture supernatants containing target antibody were harvested 6-7 days post transfection, and the target antibody was purified by affinity capture method with Protein-A resin. The purity of the target antibody was tested by SEC-HPLC (Agilent 1260 Infinity II HPLC with Welch Xtimate SEC-300 Colum, 1×PBS pH 7.4 as mobile phase) and SDS-PAGE (SurePAGE, Bis-Tris, 4-12%, Genscript, M00653).

Recombinant antibodies were successfully expressed and purified for further characterization. The proportion of main composition for each antibody was more than 90%, indicating desired high purity.

As shown in FIG. 5, A1-monoFc (PR005860) and E2-monoFc (PR005861) were tested on SDS-PAGE gel in non-reduced and reduced conditions. Results showed that purified A1-monoFc and E2-monoFc kept monomeric nature in both non-reduced and reduced conditions, indicating that their CD3 agonistic activities were attributed by crosslinking with anti-human Fc antibodies (cross-linking antibody in Example 3) instead of dimeric Fc.

### Example 5: T Cell-Mediated Cytotoxicity Bioassay for BCMA+ Cells by LDH Method

The T cell mediated cytotoxicity of BCMAxCD3 bispecific antibody A1-monoFc (PR005860) was evaluated by LDH method. Briefly, BCMA+ cells of NCI-H929 (ATCC, CRL-3580) were seeded at 3×10⁴ cells/well in 50 μL RPMI1640 medium in U-shape 96-well plates, human PBMCs were added at 3×10⁵ cells/well in 50 μL assay medium. Purified A1-monoFc (PR005860) was diluted from final concentration of 2 μg/mL by 6-fold serial dilution, and then added into plates with 50 μL/well. Each diluted sample had triplicates to calibrate experiment variations. The cytotoxicity was determined by LDH method using CytoTox 96® Non-Radioactive Cytotoxicity Assay Kit (Promega, G1780).

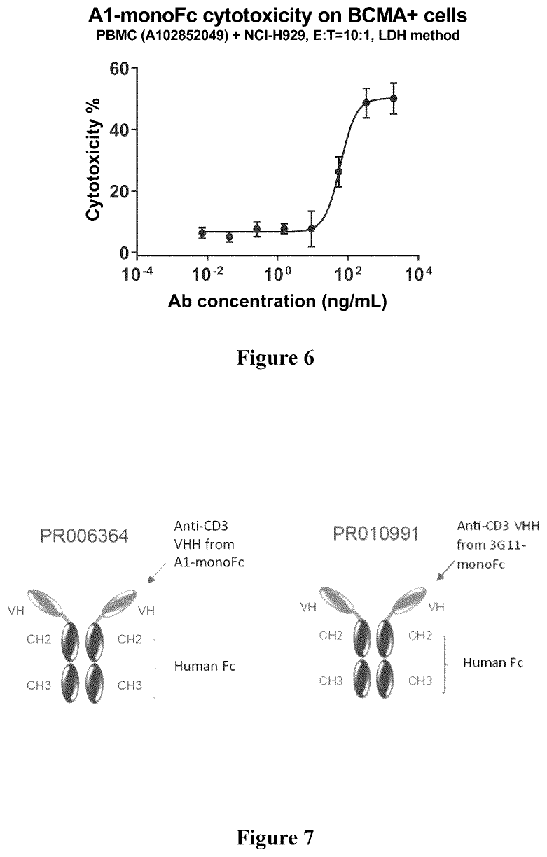

As showed in FIG. 6, PR005860 demonstrated dose-dependent cytotoxicity against NCI-H929 cells, suggesting PR005860 can activate T cells in PBMCs upon engagement of BCMA on NCI-H929 cells.

### Example 6: Binding Activity to CD3-Expressing Cells for Purified Anti-CD3 Antibodies

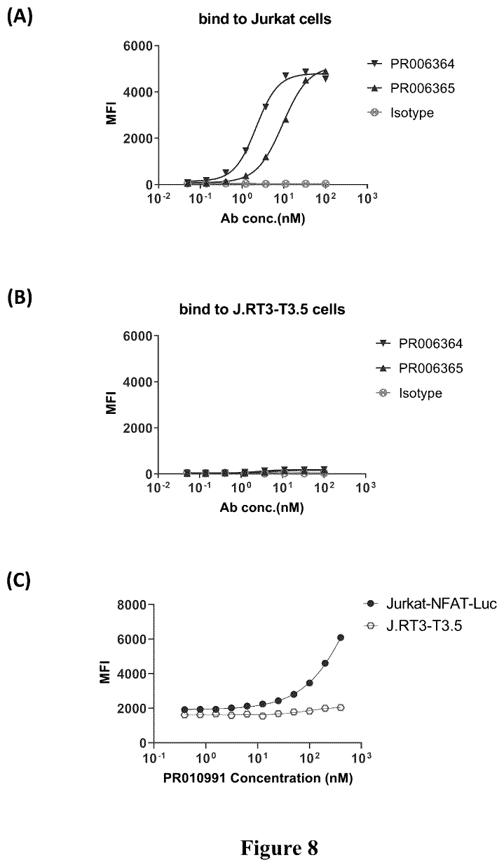

The binding activities to Jurkat and J.RT3-T3.5 cells for purified A1-HCAb (PR006364), E2-HCAb (PR006365) were determined by FACS, following the method described in Example 2. As shown in (A) and (B of FIG. 8, PR006364 and PR006365 can bind to Jurkat cells but not to J.RT3-T3.5 cells, which is consistent with the result in FIG. 1.

The binding activities to Jurkat-NFAT-Luc reporter cells and J.RT3-T3.5 cells for purified 3G11-HCAb (PR010991) were determined by FACS, following the method described in Example 2. Purified PR010991 was diluted from 400 nM to 11 different concentrations by 2-fold serial dilution. As shown in (C) of FIG. 8, PR010991 can bind to Jurkat-NFAT-Luc cells in dose-dependent manner but not to J.RT3-T3.5, suggesting PR010991 specifically recognized CD3/TCR complex on Jurkat.

### Example 7: Binding Activity to CD3 Recombinant Proteins by ELISA

The binding activities of recombinant anti-CD3 antibodies of this invention to various CD3 proteins were determined by ELISA. Since CD3/TCR complex has multiple subunits, various monomeric or heterodimeric CD3 recombinant proteins were purchased from Acro Biosystems for the ELISA assays, as listed in Table 3. The testing antibodies used in the ELISA assays are listed in Table 4.

ELISA assays were conducted as briefly described: CD3 proteins were coated onto ELISA plates at 1 μg/mL; testing antibodies OKT3 and PR006364 (A1-HCAb) were diluted from 2 μg/mL to 8 different concentrations by 3-fold serial dilution, or testing antibodies PR006370 (SP34) and PR010991 (3G11-HCAb) were diluted from 100 nM to 11 different concentrations by 2-fold serial dilution; secondary antibody (anti-human IgG Fc-HRP) was used to detect PR006364, PR006370 and PR010991, and secondary antibody (anti-mouse IgG Fc-HRP) was used to detect OKT3 binding signal. The absorption signal at 450 nm (OD450) was recorded by plate reader (Molecular Devices, SpectraMax), and the data were processed and non-linear curve fitting analyzed by GraphPad Prism 8.

TABLE 3
List of CD3 proteins as antigen used in the ELISA assays.

| Protein | Species | Catalog# | Description |
|---|---|---|---|
| hCD3e&d-llamaFc | Human | CDD-H5258 | Human CD3 |
| | | | epsilon & |
| | | | CD3 delta |
| | | | Heterodimer |
| | | | Protein fused |
| | | | with Llama Fc. |
| hCD3e&d-his | Human | CDD-H52W1 | Human CD3 |
| | | | epsilon & |
| | | | CD3 delta |
| | | | Heterodimer |
| | | | Protein with |
| | | | His Tag |
| | | | fused to |
| | | | C-terminus |
| | | | of CD3 |
| | | | epsilon chain. |
| hCD3e&g-his | Human | CDG-H52W5 | Human CD3 |
| | | | epsilon & |
| | | | CD3 gamma |
| | | | Heterodimer |
| | | | Protein with |
| | | | His Tag |
| | | | fused to |
| | | | C-terminus |
| | | | of CD3 |
| | | | epsilon chain. |
| hCD3e-his | Human | CDE-H5223 | Human CD3 |
| | | | epsilon |
| | | | Protein with |
| | | | His Tag. |
| hCD3d-his | Human | CDD-H5224 | Human CD3 delta |
| | | | Protein with |
| | | | His Tag. |
| cynoCD3e&d-his | Cynomolgus | CDD-C52W4 | Cynomolgus |
| | | | CD3 epsilon & |
| | | | CD3 delta |
| | | | Heterodimer |
| | | | Protein with |
| | | | His Tag |
| | | | fused to |
| | | | C-terminus |
| | | | of CD3 epsilon |
| | | | chain and Flag |
| | | | Tag fused to |
| | | | C-terminus |
| | | | of CD3 delta |
| | | | chain. |

TABLE 4
List of testing antibodies used in the ELISA assays.

| Antibody ID | SEQ ID NO. | Description |
|---|---|---|
| OKT3 | N/A | Anti-CD3 clone OKT3 (ThermoFisher, |
| | | 16-0037-81) |
| PR006370 | SEQ ID NOs: | Anti-CD3 clone SP34 derived |
| | (143, 168) for | mouse/human chimeric antibody in |
| | heavy, | human IgG1 Fc-silent variant (L234A, |
| | light chain | L235A, P329G). |
| PR006364 | SEQ ID NO: 141 | A1-HCAb with human IgG1 Fc-silent |
| | | variant (L234A, L235A, P329G). |
| PR010991 | SEQ ID NO: 144 | 3G11-HCAb with human IgG1 Fc-silent |
| | | variant (L234A, L235A, G237A). |

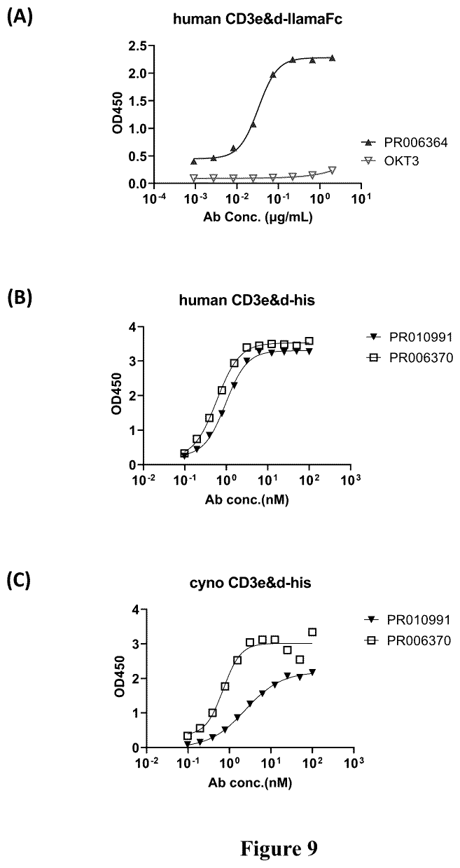

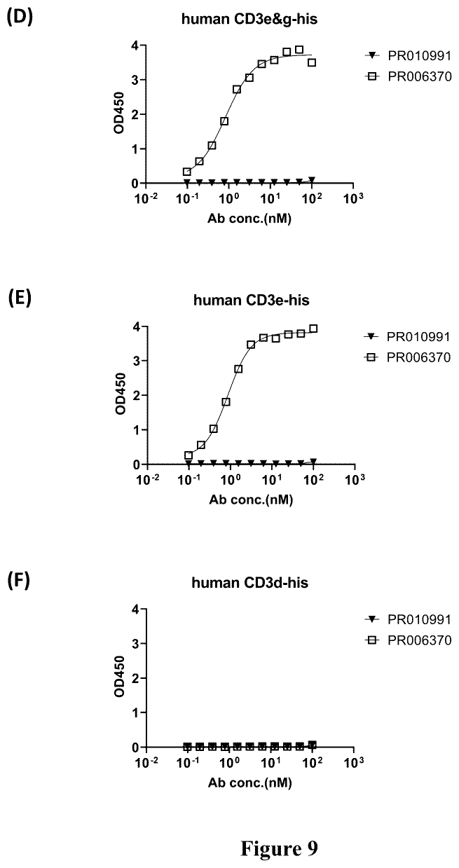

As shown in (A) of FIG. 9, OKT3 showed minimal binding to human CD3E&D heterodimer protein only at high concentration, while PR006364 (A1-HCAb) demonstrated strong and dose-dependent binding activity to human CD3E&D heterodimer protein, indicating that the binding epitope of A1 was on CD3 subunits rather than TCR alpha or beta subunits.

As shown in (B) ad (C) of FIG. 9, both PR010991 (3G11-HCAb) and PR006370 (SP34) can bind to both human CD3E&D heterodimer protein (hCD3e&d-his) and cynomolgus CD3E&D heterodimer protein (cynoCD3e&d-his). This indicates that 3G11 is anti-CD3 VHH with cross reactivity to both human and cyno CD3, same as SP34 which is well known CD3 agonistic antibody with cyno cross-reactivity. However, as shown in (D) and (E) of FIG. 9, PR006370 (SP34) can bind to both human CD3E&G heterodimer protein (hCD3e&g-his) and human CD3E monomeric protein (hCD3e-his), which is consistent with the fact that SP34's binding epitope is on the N-terminus of CD3E chain. But PR010991 (3G11-HCAb) can not bind to them, suggesting its different epitope than that of SP34.

Since 3G11 can bind to human CD3E&D heterodimer protein ((B of FIG. 9), but not to either monomeric CD3E ((E) of FIG. 9) or CD3D ((F) of FIG. 9), it would suggest that 3G11 may bind to a conformational epitope attributed from both CD3E and CD3D.

### Example 8: Generation of B7H4xCD3 Bispecific Antibodies

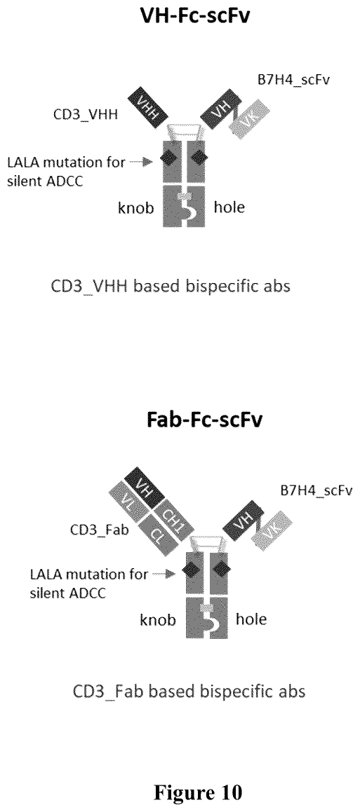

In order to evaluate if the agonistic activities of anti-CD3 can bring T cell mediated cytotoxicity, multiple B7H4xCD3 bispecific antibodies (listed in Table 5) were generated in two formats ‘Fab-Fc-scFv’ and ‘VH-Fc-scFv’ as illustrated in FIG. 10, by composing one B7H4-binding portion from anti-B7H4 PR003366 and one CD3-binding portion from various anti-CD3 of this invention or other sources. In order to form heterodimeric Fc for B7H4xCD3, “knob-into-hole” technology was employed. The “knob” mutations (S354C, T366W) were introduced to the heavy chain encoding anti-CD3 domain, and the “hole” mutations (Y349C, T366S, L368A, Y407V) were introduced to the heavy chain encoding anti-B7H4 domain. In order to abolish Fc effector functions, double mutations L234A and L235A (denoted as “LALA”) or triple mutations L234A, L235A, G237A (denoted as “AAA”) or triple mutations L234A, L235A, P329G (denoted as “AAG”) were introduced to CH2 of both heavy chains. The sequences of polypeptide chains of each B7H4xCD3 are listed in Table 6.

PR003366 is anti-B7H4 antibody in homodimeric scFv-Fc format, identified from Harbour Mice® H2L2 transgenic mouse as described in patent WO2022002012A1, and its sequences are listed in Table 7.

PR001848 and PR003886 are humanized variants derived from anti-CD3 clone SP34 (PR006370) with different binding activities, as described in patent WO2021063330A1, and their sequences are listed in Table 8. Mutations “LALA” or “AAG” were introduced to remove Fc effector functions.

PR006361 is anti-CD3 HCAb with human IgG1 Fc by inserting anti-CD3 VHH originated from patent WO2016180982A1 into pTT5-VHH-hFc vector, and its sequences are listed in Table 8. Mutations C220S and “AAG” were introduced to PR006361.

The recombinant B7H4xCD3 bispecific antibody was produced by transfecting the plasmids encoding the polypeptide chains of each antibody into mammalian host cells, followed by affinity capture method or other purification method as described in Example 4. The expression yield and purity determined by SEC-HPLC of B7H4xCD3 bispecific antibodies are listed in Table 9.

TABLE 5
Generation of B7H4×CD3 bispecific antibodies.

| | | | | Source of | Fc-silent |
|---|---|---|---|---|---|
| Format | B7H4×CD3 | Anti-B7H4 | Anti-CD3 | anti-CD3 | (mutations) |
| Fab-Fc-scFv | PR003733 | PR003366 | PR001848 | Humanized SP34, | hIgG1 |
| | | | | originated | (L234A, |
| | | | | from patent | L235A) |
| | | | | WO2021063330A1. | |
| Fab-Fc-scFv | PR003899 | PR003366 | PR003886 | Humanized SP34, | hIgG1 |
| | | | | originated | (L234A, |
| | | | | from patent | L235A) |
| | | | | WO2021063330A1. | |
| VH-Fc-scFv | PR006001 | PR003366 | PR006361 | Anti-CD3/TCR | hIgG1 |
| | | | | VHH, originated | (L234A, |
| | | | | from patent | L235A) |
| | | | | WO2016180982A1. | |
| VH-Fc-scFv | PR006002 | PR003366 | PR006364 | A1-HCAb of | hIgG1 |
| | | | | this invention. | (L234A, |
| | | | | | L235A) |
| VH-Fc-scFv | PR006003 | PR003366 | PR006365 | E2-HCAb of | hIgG1 |
| | | | | this invention. | (L234A, |
| | | | | | L235A) |
| VH-Fc-scFv | PR008551 | PR003366 | PR010991 | | hIgG1 |
| | | | | 3G11-HCAb of | (L234A, |
| | | | | this invention. | L235A, |
| | | | | | G237A) |

TABLE 6
Polypeptide chain sequences of B7H4xCD3 bispecific antibodies.

| | | anti-B7H4xCD3 Fab(PR001848)-Fc-scFv(PR003366), |
|---|---|---|
| SEQ ID NO: | PR003733 | hIgG1(KiH, LALA) |
| 169 | anti-CD3 heavy chain | EVQLLESGGGLVQPGGSLRLSCAASGFTFSTYAMN |
| | | WVRQAPGKGLEWVSRIRSKYNNYATYYADSVKD |
| | | RFTISRDDSKSTLYLQMNSLRAEDTAVYYCVRHGN |
| | | FGNSYVSWFAYWGQGTLVTVSSASTKGPSVFPLAP |
| | | SSKSTSGGTAALGCLVKDYFPEPVTVSWNSGALTS |
| | | GVHTFPAVLQSSGLYSLSSVVTVPSSSLGTQTYICN |
| | | VNHKPSNTKVDKKVEPKSCDKTHTCPPCPAPEAAG |
| | | GPSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKAK |
| | | GQPREPQVYTLPPCREEMTKNQVSLWCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| 167 | anti-CD3 light chain | QAVVTQEPSLTVSPGGTVTLTCRSSTGAVTTSNYA |
| | | NWVQQKPGQAPRGLIGGTNKRAPWTPARFSGSLL |
| | | GDKAALTLLGAQPEDEAEYFCALWYSNLWVFGGG |
| | | TKLTVLGQPKAAPSVTLFPPSSEELQANKATLVCLI |
| | | SDFYPGAVTVAWKADSSPVKAGVETTTPSKQSNN |
| | | KYAASSYLSLTPEQWKSHRSYSCQVTHEGSTVEKT |
| | | VAPTECS |
| 170 | anti-B7H4 scFv-Fc chain | EIVMTQSPASLSVSPGERATLSCRASQSVSSNLAWY |
| | | QQKPGQAPRLLIYGASTRATGIPARVSGGGSGTEFT |
| | | LTISSLQSEDFAVYYCQQYKNWPFTFGPGTKLEIKG |
| | | GGGSGGGGSGGGGSGGGGSQVQLVQSGAEVKKP |
| | | GSSMRVSCKASEDTFSSYAISWVRQAPGQGLEWM |
| | | GGTAPIFGTTNYAQKFQGRVTITADKSTSTAYMEL |
| | | SSLRSEDTAVYYCARGGPYFDYWGQGTLVTVSSA |
| | | SEPKSSDKTHTCPPCPAPEAAGGPSVFLFPPKPKDT |
| | | LMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVCTLPPS |
| | | REEMTKNQVSLSCAVKGFYPSDIAVEWESNGQPEN |
| | | NYKTTPPVLDSDGSFFLVSKLTVDKSRWQQGNVFS |
| | | CSVMHEALHNHYTQKSLSLSPGK |
| | | anti-B7H4xCD3 Fab(PR003886)-Fc-scFv(PR003366), |
| SEQ ID NO: | PR003899 | hIgG1(KIH, LALA) |
| 171 | anti-CD3 heavy chain | EVQLVESGGGLVQPGGSLKLSCAASGFTFSTYAMN |
| | | WVRQASGKGLEWVGRIRSKYNNYATYYADSVKD |
| | | RFTISRDDSKNTAYLQMNSLKTEDTAVYYCTRHGN |
| | | FGNSYVSWFAYWGQGTLVTVSSASTKGPSVFPLAP |
| | | SSKSTSGGTAALGCLVKDYFPEPVTVSWNSGALTS |
| | | GVHTFPAVLQSSGLYSLSSVVTVPSSSLGTQTYICN |
| | | VNHKPSNTKVDKKVEPKSCDKTHTCPPCPAPEAAG |
| | | GPSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKAK |
| | | GQPREPQVYTLPPCREEMTKNQVSLWCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| 167 | anti-CD3 light chain | QAVVTQEPSLTVSPGGTVTLTCRSSTGAVTTSNYA |
| | | NWVQQKPGQAPRGLIGGTNKRAPWTPARFSGSLL |
| | | GDKAALTLLGAQPEDEAEYFCALWYSNLWVFGGG |
| | | TKLTVLGQPKAAPSVTLFPPSSEELQANKATLVCLI |
| | | SDFYPGAVTVAWKADSSPVKAGVETTTPSKQSNN |
| | | KYAASSYLSLTPEQWKSHRSYSCQVTHEGSTVEKT |
| | | VAPTECS |
| 170 | anti-B7H4 scFv-Fc chain | EIVMTQSPASLSVSPGERATLSCRASQSVSSNLAWY |
| | | QQKPGQAPRLLIYGASTRATGIPARVSGGGSGTEFT |
| | | LTISSLQSEDFAVYYCQQYKNWPFTFGPGTKLEIKG |
| | | GGGSGGGGSGGGGSGGGGSQVQLVQSGAEVKKP |
| | | GSSMRVSCKASEDTFSSYAISWVRQAPGQGLEWM |
| | | GGTAPIFGTTNYAQKFQGRVTITADKSTSTAYMEL |
| | | SSLRSEDTAVYYCARGGPYFDYWGQGTLVTVSSA |
| | | SEPKSSDKTHTCPPCPAPEAAGGPSVFLFPPKPKDT |
| | | LMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVCTLPPS |
| | | REEMTKNQVSLSCAVKGFYPSDIAVEWESNGQPEN |
| | | NYKTTPPVLDSDGSFFLVSKLTVDKSRWQQGNVFS |
| | | CSVMHEALHNHYTQKSLSLSPGK |
| | | anti-B7H4xCD3 VH(PR006361)-Fc-scFv(PR003366), |
| SEQ ID NO: | PR006001 | hIgG1(dCH1, KiH, LALA) |
| 174 | anti-CD3 VH-Fc chain | EVQLVESGGGPVQAGGSLRLSCAASGRTYRGYSM |
| | | GWFRQAPGKEREFVAAIVWSGGNTYYEDSVKGRF |
| | | TISRDNAKNTMYLQMTSLKPEDSATYYCAAKIRPY |
| | | IFKIAGQYDYWGQGTQVTVSSASEPKSSDKTHTCP |
| | | PCPAPEAAGGPSVFLFPPKPKDTLMISRTPEVTCVV |
| | | VDVSHEDPEVKFNWYVDGVEVHNAKTKPREEQY |
| | | NSTYRVVSVLTVLHQDWLNGKEYKCKVSNKALPA |
| | | PIEKTISKAKGQPREPQVYTLPPCREEMTKNQVSLW |
| | | CLVKGFYPSDIAVEWESNGQPENNYKTTPPVLDSD |
| | | GSFFLYSKLTVDKSRWQQGNVFSCSVMHEALHNH |
| | | YTQKSLSLSPGK |
| 170 | anti-B7H4 scFv-Fc chain | EIVMTQSPASLSVSPGERATLSCRASQSVSSNLAWY |
| | | QQKPGQAPRLLIYGASTRATGIPARVSGGGSGTEFT |
| | | LTISSLQSEDFAVYYCQQYKNWPFTFGPGTKLEIKG |
| | | GGGSGGGGSGGGGSGGGGSQVQLVQSGAEVKKP |
| | | GSSMRVSCKASEDTFSSYAISWVRQAPGQGLEWM |
| | | GGTAPIFGTTNYAQKFQGRVTITADKSTSTAYMEL |
| | | SSLRSEDTAVYYCARGGPYFDYWGQGTLVTVSSA |
| | | SEPKSSDKTHTCPPCPAPEAAGGPSVFLFPPKPKDT |
| | | LMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVCTLPPS |
| | | REEMTKNQVSLSCAVKGFYPSDIAVEWESNGQPEN |
| | | NYKTTPPVLDSDGSFFLVSKLTVDKSRWQQGNVFS |
| | | CSVMHEALHNHYTQKSLSLSPGK |
| | | anti-B7H4xCD3 VH(A1)-Fc-scFv(PR003366), |
| SEQ ID NO: | PR006002 | hIgG1(dCH1, KiH, LALA) |
| 175 | anti-CD3 VH-Fc chain | RIRLVESGGGLVQAGGSLRLSCAASGRTSGIPAMA |
| | | WFRQAPGKEREFVAMAWSGGGTIYGDSVKNRFTI |
| | | SRDNTQNTIFLQMNSLKPEDTALYYCAAGRSTSNR |
| | | ADQYNYWGQGTQVTVSSASEPKSSDKTHTCPPCP |
| | | APEAAGGPSVFLFPPKPKDTLMISRTPEVTCVVVDV |
| | | SHEDPEVKFNWYVDGVEVHNAKTKPREEQYNSTY |
| | | RVVSVLTVLHQDWLNGKEYKCKVSNKALPAPIEK |
| | | TISKAKGQPREPQVYTLPPCREEMTKNQVSLWCLV |
| | | KGFYPSDIAVEWESNGQPENNYKTTPPVLDSDGSF |
| | | FLYSKLTVDKSRWQQGNVFSCSVMHEALHNHYTQ |
| | | KSLSLSPGK |
| 170 | anti-B7H4 scFv-Fc chain | EIVMTQSPASLSVSPGERATLSCRASQSVSSNLAWY |
| | | QQKPGQAPRLLIYGASTRATGIPARVSGGGSGTEFT |
| | | LTISSLQSEDFAVYYCQQYKNWPFTFGPGTKLEIKG |
| | | GGGSGGGGSGGGGSGGGGSQVQLVQSGAEVKKP |
| | | GSSMRVSCKASEDTFSSYAISWVRQAPGQGLEWM |
| | | GGTAPIFGTTNYAQKFQGRVTITADKSTSTAYMEL |
| | | SSLRSEDTAVYYCARGGPYFDYWGQGTLVTVSSA |
| | | SEPKSSDKTHTCPPCPAPEAAGGPSVFLFPPKPKDT |
| | | LMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVCTLPPS |
| | | REEMTKNQVSLSCAVKGFYPSDIAVEWESNGQPEN |
| | | NYKTTPPVLDSDGSFFLVSKLTVDKSRWQQGNVFS |
| | | CSVMHEALHNHYTQKSLSLSPGK |
| | | anti-B7H4xCD3 VH(E2)-Fc-scFv(PR003366), |
| SEQ ID NO: | PR006003 | hIgG1(dCH1, KiH, LALA) |
| 176 | anti-CD3 VH-Fc chain | EVQLVESGGGLVQAGGSLRLSCAASGRTNGIPAMA |
| | | WFRQAPGKEREFVAMAWSGGGTIYGDSVKNRFTI |
| | | SRNNAQNTISLQMSSLRPEDTALYFCAAGRTTSNK |
| | | ADHYNYWGQGTQVTVSSASEPKSSDKTHTCPPCP |
| | | APEAAGGPSVFLFPPKPKDTLMISRTPEVTCVVVDV |
| | | SHEDPEVKFNWYVDGVEVHNAKTKPREEQYNSTY |
| | | RVVSVLTVLHQDWLNGKEYKCKVSNKALPAPIEK |
| | | TISKAKGQPREPQVYTLPPCREEMTKNQVSLWCLV |
| | | KGFYPSDIAVEWESNGQPENNYKTTPPVLDSDGSF |
| | | FLYSKLTVDKSRWQQGNVFSCSVMHEALHNHYTQ |
| | | KSLSLSPGK |
| 170 | anti-B7H4 scFv-Fc chain | EIVMTQSPASLSVSPGERATLSCRASQSVSSNLAWY |
| | | QQKPGQAPRLLIYGASTRATGIPARVSGGGSGTEFT |
| | | LTISSLQSEDFAVYYCQQYKNWPFTFGPGTKLEIKG |
| | | GGGSGGGGSGGGGSGGGGSQVQLVQSGAEVKKP |
| | | GSSMRVSCKASEDTFSSYAISWVRQAPGQGLEWM |
| | | GGTAPIFGTTNYAQKFQGRVTITADKSTSTAYMEL |
| | | SSLRSEDTAVYYCARGGPYFDYWGQGTLVTVSSA |
| | | SEPKSSDKTHTCPPCPAPEAAGGPSVFLFPPKPKDT |
| | | LMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVCTLPPS |
| | | REEMTKNQVSLSCAVKGFYPSDIAVEWESNGQPEN |
| | | NYKTTPPVLDSDGSFFLVSKLTVDKSRWQQGNVFS |
| | | CSVMHEALHNHYTQKSLSLSPGK |
| | | anti-B7H4xCD3 VH(3G11)-Fc-scFv(PR003366), |
| SEQ ID NO: | PR008551 | hIgG1(dCH1, KiH, AAA) |
| 177 | anti-CD3 VH-Fc chain | RVHLAESGGGLVQAGGSLRLSCAASGSRLNDNAM |
| | | AWFRQAPGKEREFVGSIFWTGGNTYYADAVKGRL |
| | | TISRDNAKNALYLQMNSLKPEDTAVYYCAASRVV |
| | | WYYCSGYGCSRAGDYVYWGQGTQVTVSSASEPK |
| | | SSDKTHTCPPCPAPEAAGAPSVFLFPPKPKDTLMIS |
| | | RTPEVTCVVVDVSHEDPEVKFNWYVDGVEVHNA |
| | | KTKPREEQYNSTYRVVSVLTVLHQDWLNGKEYKC |
| | | KVSNKALPAPIEKTISKAKGQPREPQVYTLPPCREE |
| | | MTKNQVSLWCLVKGFYPSDIAVEWESNGQPENNY |
| | | KTTPPVLDSDGSFFLYSKLTVDKSRWQQGNVFSCS |
| | | VMHEALHNHYTQKSLSLSPGK |
| 178 | anti-B7H4 scFv-Fc chain | EIVMTQSPASLSVSPGERATLSCRASQSVSSNLAWY |
| | | QQKPGQAPRLLIYGASTRATGIPARVSGGGSGTEFT |
| | | LTISSLQSEDFAVYYCQQYKNWPFTFGPGTKLEIKG |
| | | GGGSGGGGSGGGGSGGGGSQVQLVQSGAEVKKP |
| | | GSSMRVSCKASEDTFSSYAISWVRQAPGQGLEWM |
| | | GGTAPIFGTTNYAQKFQGRVTITADKSTSTAYMEL |
| | | SSLRSEDTAVYYCARGGPYFDYWGQGTLVTVSSA |
| | | SEPKSSDKTHTCPPCPAPEAAGAPSVFLFPPKPKDT |
| | | LMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVCTLPPS |
| | | REEMTKNQVSLSCAVKGFYPSDIAVEWESNGQPEN |
| | | NYKTTPPVLDSDGSFFLVSKLTVDKSRWQQGNVFS |
| | | CSVMHEALHNHYTQKSLSLSPGK |

TABLE 7
Sequences of anti-B7H4 PR003366.

| SEQ ID NO: | PR003366 | anti-B7H4 scFv(L4H)-hFc |
|---|---|---|
| 202 | PR003366 scFv-Fc | EIVMTQSPASLSVSPGERATLSCRASQSVSSNLAWY |
| | Sequence | QQKPGQAPRLLIYGASTRATGIPARVSGGGSGTEFT |
| | | LTISSLQSEDFAVYYCQQYKNWPFTFGPGTKLEIKG |
| | | GGGSGGGGSGGGGSGGGGSQVQLVQSGAEVKKP |
| | | GSSMRVSCKASEDTFSSYAISWVRQAPGQGLEWM |
| | | GGTAPIFGTTNYAQKFQGRVTITADKSTSTAYMEL |
| | | SSLRSEDTAVYYCARGGPYFDYWGQGTLVTVSSG |
| | | GGASEPKSCDKTHTCPPCPAPELLGGPSVFLFPPKP |
| | | KDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYT |
| | | LPPSREEMTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 134 | PR003366 VL Sequence | EIVMTQSPASLSVSPGERATLSCRASQSVSSNLAWY |
| | | QQKPGQAPRLLIYGASTRATGIPARVSGGGSGTEFT |
| | | LTISSLQSEDFAVYYCQQYKNWPFTFGPGTKLEIK |
| 85 | PR003366 VL FWR1 | EIVMTQSPASLSVSPGERATLSC |
| | (Chothia) | |
| 88 | PR003366 VL CDR1 | RASQSVSSNLA |
| | (Chothia) | |
| 90 | PR003366 VL FWR2 | WYQQKPGQAPRLLIY |
| | (Chothia) | |
| 93 | PR003366 VL CDR2 | GASTRAT |
| | (Chothia) | |
| 95 | PR003366 VL FWR3 | GIPARVSGGGSGTEFTLTISSLQSEDFAVYYC |
| | (Chothia) | |
| 98 | PR003366 VL CDR3 | QQYKNWPFT |
| | (Chothia) | |
| 100 | PR003366 VL FWR4 | FGPGTKLEIK |
| | (Chothia) | |
| 103 | PR003366 VH Sequence | QVQLVQSGAEVKKPGSSMRVSCKASEDTFSSYAIS |
| | | WVRQAPGOGLEWMGGTAPIFGTTNYAQKFQGRV |
| | | TITADKSTSTAYMELSSLRSEDTAVYYCARGGPYF |
| | | DYWGQGTLVTVSS |
| 3 | PR003366 VH FWR1 | QVQLVQSGAEVKKPGSSMRVSCKAS |
| | (Chothia) | |
| 14 | PR003366 VH CDR1 | EDTFSSY |
| | (Chothia) | |
| 25 | PR003366 VH FWR2 | AISWVRQAPGQGLEWMGGT |
| | (Chothia) | |
| 38 | PR003366 VH CDR2 | APIFGT |
| | (Chothia) | |
| 46 | PR003366 VH FWR3 | TNYAQKFQGRVTITADKSTSTAYMELSSLRSEDTA |
| | (Chothia) | VYYCAR |
| 59 | PR003366 VH CDR3 | GGPYFDY |
| | (Chothia) | |
| 81 | PR003366 VH FWR4 | WGQGTLVTVSS |
| | (Chothia) | |

TABLE 8
Sequences of anti-CD3 PR006370, PR001848, PR003886 and PR006361.

| SEQ ID NO: | PR006370 | anti-CD3 SP34 chimeric hIgG1(AAG) |
|---|---|---|
| 143 | PR006370 Heavy Chain | EVQLVESGGGLVQPKGSLKLSCAASGFTFNTYAMNW |
| | Sequence | VRQAPGKGLEWVARIRSKYNNYATYYADSVKDRFTI |
| | | SRDDSQSILYLQMNNLKTEDTAMYYCVRHGNFGNSY |
| | | VSWFAYWGQGTLVTVSAASTKGPSVFPLAPSSKSTSG |
| | | GTAALGCLVKDYFPEPVTVSWNSGALTSGVHTFPAVL |
| | | QSSGLYSLSSVVTVPSSSLGTQTYICNVNHKPSNTKVD |
| | | KKVEPKSCDKTHTCPPCPAPEAAGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALGAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPGK |
| 109 | PR006370 VH Sequence | EVQLVESGGGLVQPKGSLKLSCAASGFTFNTYAMNW |
| | | VRQAPGKGLEWVARIRSKYNNYATYYADSVKDRFTI |
| | | SRDDSQSILYLQMNNLKTEDTAMYYCVRHGNFGNSY |
| | | VSWFAYWGQGTLVTVSA |
| 9 | PR006370 VH FWR1 | EVQLVESGGGLVQPKGSLKLSCAAS |
| | (Chothia) | |
| 19 | PR006370 VH CDR1 | GFTFNTY |
| | (Chothia) | |
| 30 | PR006370 VH FWR2 | AMNWVRQAPGKGLEWVARI |
| | (Chothia) | |
| 37 | PR006370 VH CDR2 | RSKYNNYA |
| | (Chothia) | |
| 52 | PR006370 VH FWR3 | TYYADSVKDRFTISRDDSQSILYLQMNNLKTEDTAMY |
| | (Chothia) | YCVR |
| 58 | PR006370 VH CDR3 | HGNFGNSYVSWFAY |
| | (Chothia) | |
| 83 | PR006370 VH FWR4 | WGQGTLVTVSA |
| | (Chothia) | |
| 168 | PR006370 Light Chain | QAVVTQESALTTSPGETVTLTCRSSTGAVTTSNYANW |
| | Sequence | VQEKPDHLFTGLIGGTNKRAPGVPARFSGSLIGDKAA |
| | | LTITGAQTEDEAIYFCALWYSNLWVFGGGTKLTVLGQ |
| | | PKAAPSVTLFPPSSEELQANKATLVCLISDFYPGAVTV |
| | | AWKADSSPVKAGVETTTPSKQSNNKYAASSYLSLTPE |
| | | QWKSHRSYSCQVTHEGSTVEKTVAPTECS |
| 135 | PR006370 VL Sequence | QAVVTQESALTTSPGETVTLTCRSSTGAVTTSNYANW |
| | | VQEKPDHLFTGLIGGTNKRAPGVPARFSGSLIGDKAA |
| | | LTITGAQTEDEAIYFCALWYSNLWVFGGGTKLTVL |
| 86 | PR006370 VL FWR1 | QAVVTQESALTTSPGETVTLTC |
| | (Chothia) | |
| 87 | PR006370 VL CDR1 | RSSTGAVTTSNYAN |
| | (Chothia) | |
| 91 | PR006370 VL FWR2 | WVQEKPDHLFTGLIG |
| | (Chothia) | |
| 92 | PR006370 VL CDR2 | GTNKRAP |
| | (Chothia) | |
| 96 | PR006370 VL FWR3 | GVPARFSGSLIGDKAALTITGAQTEDEAIYFC |
| | (Chothia) | |
| 97 | PR006370 VL CDR3 | ALWYSNLWV |
| | (Chothia) | |
| 99 | PR006370 VL FWR4 | FGGGTKLTVL |
| | (Chothia) | |
| SEQ ID NO: | PR001848 | anti-CD3 humanized SP34 hIgG1(AAG) |
| 137 | PR001848 Heavy Chain | EVQLLESGGGLVQPGGSLRLSCAASGFTFSTYAMNW |
| | Sequence | VRQAPGKGLEWVSRIRSKYNNYATYYADSVKDRFTIS |
| | | RDDSKSTLYLQMNSLRAEDTAVYYCVRHGNFGNSYV |
| | | SWFAYWGQGTLVTVSSASTKGPSVFPLAPSSKSTSGG |
| | | TAALGCLVKDYFPEPVTVSWNSGALTSGVHTFPAVLQ |
| | | SSGLYSLSSVVTVPSSSLGTQTYICNVNHKPSNTKVDK |
| | | KVEPKSCDKTHTCPPCPAPEAAGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALGAPIEKTISKAKGQPREPQVYTLPPSREEMTKN |
| | | QVSLTCLVKGFYPSDIAVEWESNGQPENNYKTTPPVL |
| | | DSDGSFFLYSKLTVDKSRWQQGNVFSCSVMHEALHN |
| | | HYTQKSLSLSPGK |
| 102 | PR001848 VH Sequence | EVQLLESGGGLVQPGGSLRLSCAASGFTFSTYAMNW |
| | | VRQAPGKGLEWVSRIRSKYNNYATYYADSVKDRFTIS |
| | | RDDSKSTLYLQMNSLRAEDTAVYYCVRHGNFGNSYV |
| | | SWFAYWGQGTLVTVSS |
| 2 | PR001848 VH FWR1 | EVQLLESGGGLVQPGGSLRLSCAAS |
| | (Chothia) | |
| 13 | PR001848 VH CDR1 | GFTFSTY |
| | (Chothia) | |
| 24 | PR001848 VH FWR2 | AMNWVRQAPGKGLEWVSRI |
| | (Chothia) | |
| 37 | PR001848 VH CDR2 | RSKYNNYA |
| | (Chothia) | |
| 45 | PR001848 VH FWR3 | TYYADSVKDRFTISRDDSKSTLYLQMNSLRAEDTAVY |
| | (Chothia) | YCVR |
| 58 | PR001848 VH CDR3 | HGNFGNSYVSWFAY |
| | (Chothia) | |
| 81 | PR001848 VH FWR4 | WGQGTLVTVSS |
| | (Chothia) | |
| 167 | PR001848 Light Chain | QAVVTQEPSLTVSPGGTVTLTCRSSTGAVTTSNYANW |
| | Sequence | VQQKPGQAPRGLIGGTNKRAPWTPARFSGSLLGDKA |
| | | ALTLLGAQPEDEAEYFCALWYSNLWVFGGGTKLTVL |
| | | GQPKAAPSVTLFPPSSEELQANKATLVCLISDFYPGAV |
| | | TVAWKADSSPVKAGVETTTPSKQSNNKYAASSYLSLT |
| | | PEQWKSHRSYSCQVTHEGSTVEKTVAPTECS |
| 133 | PR001848 VL Sequence | QAVVTQEPSLTVSPGGTVTLTCRSSTGAVTTSNYANW |
| | | VQQKPGQAPRGLIGGTNKRAPWTPARFSGSLLGDKA |
| | | ALTLLGAQPEDEAEYFCALWYSNLWVFGGGTKLTVL |
| 84 | PR001848 VL FWR1 | QAVVTQEPSLTVSPGGTVTLTC |
| | (Chothia) | |
| 87 | PR001848 VL CDR1 | RSSTGAVTTSNYAN |
| | (Chothia) | |
| 89 | PR001848 VL FWR2 | WVQQKPGQAPRGLIG |
| | (Chothia) | |
| 92 | PR001848 VL CDR2 | GTNKRAP |
| | (Chothia) | |
| 94 | PR001848 VL FWR3 | WTPARFSGSLLGDKAALTLLGAQPEDEAEYFC |
| | (Chothia) | |
| 97 | PR001848 VL CDR3 | ALWYSNLWV |
| | (Chothia) | |
| 99 | PR001848 VL FWR4 | FGGGTKLTVL |
| | (Chothia) | |
| SEQ ID NO: | PR003886 | anti-CD3 humanized SP34 hIgG1(LALA) |
| 138 | PR003886 Heavy Chain | EVQLVESGGGLVQPGGSLKLSCAASGFTFSTYAMNW |
| | Sequence | VRQASGKGLEWVGRIRSKYNNYATYYADSVKDRFTI |
| | | SRDDSKNTAYLQMNSLKTEDTAVYYCTRHGNFGNSY |
| | | VSWFAYWGQGTLVTVSSASTKGPSVFPLAPSSKSTSG |
| | | GTAALGCLVKDYFPEPVTVSWNSGALTSGVHTFPAVL |
| | | QSSGLYSLSSVVTVPSSSLGTQTYICNVNHKPSNTKVD |
| | | KKVEPKSCDKTHTCPPCPAPEAAGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALPAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPGK |
| 104 | PR003886 VH Sequence | EVQLVESGGGLVQPGGSLKLSCAASGFTFSTYAMNW |
| | | VRQASGKGLEWVGRIRSKYNNYATYYADSVKDRFTI |
| | | SRDDSKNTAYLQMNSLKTEDTAVYYCTRHGNFGNSY |
| | | VSWFAYWGQGTLVTVSS |
| 4 | PR003886 VH FWR1 | EVQLVESGGGLVQPGGSLKLSCAAS |
| | (Chothia) | |
| 13 | PR003886 VH CDR1 | GFTFSTY |
| | (Chothia) | |
| 26 | PR003886 VH FWR2 | AMNWVRQASGKGLEWVGRI |
| | (Chothia) | |
| 37 | PR003886 VH CDR2 | RSKYNNYA |
| | (Chothia) | |
| 47 | PR003886 VH FWR3 | TYYADSVKDRFTISRDDSKNTAYLQMNSLKTEDTAV |
| | (Chothia) | YYCTR |
| 58 | PR003886 VH CDR3 | HGNFGNSYVSWFAY |
| | (Chothia) | |
| 81 | PR003886 VH FWR4 | WGQGTLVTVSS |
| | (Chothia) | |
| 167 | PR003886 Light Chain | QAVVTQEPSLTVSPGGTVTLTCRSSTGAVTTSNYANW |
| | Sequence | VQQKPGQAPRGLIGGTNKRAPWTPARFSGSLLGDKA |
| | | ALTLLGAQPEDEAEYFCALWYSNLWVFGGGTKLTVL |
| | | GQPKAAPSVTLFPPSSEELQANKATLVCLISDFYPGAV |
| | | TVAWKADSSPVKAGVETTTPSKQSNNKYAASSYLSLT |
| | | PEQWKSHRSYSCQVTHEGSTVEKTVAPTECS |
| 133 | PR003886 VL Sequence | QAVVTQEPSLTVSPGGTVTLTCRSSTGAVTTSNYANW |
| | | VQQKPGQAPRGLIGGTNKRAPWTPARFSGSLLGDKA |
| | | ALTLLGAQPEDEAEYFCALWYSNLWVFGGGTKLTVL |
| 84 | PR003886 VL FWR1 | QAVVTQEPSLTVSPGGTVTLTC |
| | (Chothia) | |
| 87 | PR003886 VL CDR1 | RSSTGAVTTSNYAN |
| | (Chothia) | |
| 89 | PR003886 VL FWR2 | WVQQKPGQAPRGLIG |
| | (Chothia) | |
| 92 | PR003886 VL CDR2 | GTNKRAP |
| | (Chothia) | |
| 94 | PR003886 VL FWR3 | WTPARFSGSLLGDKAALTLLGAQPEDEAEYFC |
| | (Chothia) | |
| 97 | PR003886 VL CDR3 | ALWYSNLWV |
| | (Chothia) | |
| 99 | PR003886 VL FWR4 | FGGGTKLTVL |
| | (Chothia) | |
| | | anti-CD3/TCR HCAb hIgG1(AAG) from |
| SEQ ID NO: | PR006361 | WO2016180982A1 |
| 140 | PR006361 Heavy Chain | EVQLVESGGGPVQAGGSLRLSCAASGRTYRGYSMGW |
| | Sequence | FRQAPGKEREFVAAIVWSGGNTYYEDSVKGRFTISRD |
| | | NAKNTMYLQMTSLKPEDSATYYCAAKIRPYIFKIAGQ |
| | | YDYWGQGTQVTVSSASEPKSSDKTHTCPPCPAPEAAG |
| | | GPSVFLFPPKPKDTLMISRTPEVTCVVVDVSHEDPEVK |
| | | FNWYVDGVEVHNAKTKPREEQYNSTYRVVSVLTVLH |
| | | QDWLNGKEYKCKVSNKALGAPIEKTISKAKGQPREPQ |
| | | VYTLPPSREEMTKNQVSLTCLVKGFYPSDIAVEWESN |
| | | GQPENNYKTTPPVLDSDGSFFLYSKLTVDKSRWQQG |
| | | NVFSCSVMHEALHNHYTQKSLSLSPGK |
| 106 | PR006361 VH Sequence | EVQLVESGGGPVQAGGSLRLSCAASGRTYRGYSMGW |
| | | FRQAPGKEREFVAAIVWSGGNTYYEDSVKGRFTISRD |
| | | NAKNTMYLQMTSLKPEDSATYYCAAKIRPYIFKIAGQ |
| | | YDYWGQGTQVTVSS |
| 6 | PR006361 VH FWR1 | EVQLVESGGGPVQAGGSLRLSCAAS |
| | (Chothia) | |
| 16 | PR006361 VH CDR1 | GRTYRGY |
| | (Chothia) | |
| 28 | PR006361 VH FWR2 | SMGWFRQAPGKEREFVAAI |
| | (Chothia) | |
| 40 | PR006361 VH CDR2 | VWSGGN |
| | (Chothia) | |
| 49 | PR006361 VH FWR3 | TYYEDSVKGRFTISRDNAKNTMYLQMTSLKPEDSATY |
| | (Chothia) | YCAA |
| 61 | PR006361 VH CDR3 | KIRPYIFKIAGQYDY |
| | (Chothia) | |
| 82 | PR006361 VH FWR4 | WGQGTQVTVSS |
| | (Chothia) | |

TABLE 9
Antibody production summary for B7H4×CD3.

| | | | | | SEC |
|---|---|---|---|---|---|
| | | | | Yield | purity |
| Format | B7H4×CD3 | Anti-B7H4 | Anti-CD3 | (mg/L) | (%) |
| Fab-Fc-scFv | PR003733 | PR003366 | PR001848 | 171 | 99.28 |
| Fab-Fc-scFv | PR003899 | PR003366 | PR003886 | 180 | 99.17 |
| VH-Fc-scFv | PR006001 | PR003366 | PR006361 | 111 | 100 |
| VH-Fc-scFv | PR006002 | PR003366 | PR006364 | 80 | 100 |
| VH-Fc-scFv | PR006003 | PR003366 | PR006365 | 52 | 94 |
| VH-Fc-scFv | PR008551 | PR003366 | PR010991 | 205 | 100 |

### Example 9: T Cell-Mediated Cytotoxicity of B7H4xCD3 Bispecific Antibodies Determined by RTCA Method

The T cell mediated cytotoxicity of B7H4xCD3 bispecific antibodies to specific target cells were determined by RTCA (Real-time quantitative cell analysis) method. Briefly, the target cells, specifically, the tumor cells MDA-MB-468 (ATCC, HTB-132) with high B7H4 expression, were seeded at 2×10⁴ cells/well in 50 μL assay medium; and the effector cells, specifically, human PBMCs or isolated pan T cells, were seeded at 2×10⁵ cells/well in 50 μL assay medium. Then diluted testing bispecific antibodies were added into wells in 50 μL assay medium. The assay plates were incubated at 37° C., 5% CO₂ for 24 hours, followed by real-time monitoring on Agilent xCELLigence RTCA instrument. The data were recorded and processed with the protocol provided by the vendor, and the results were analyzed and presented by GraphPad Prism 8.

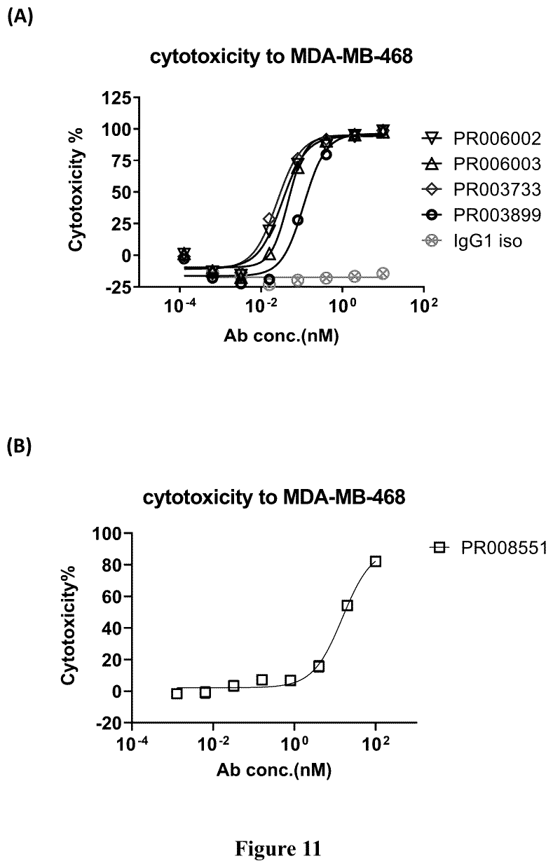

B7H4xCD3 bispecific antibodies (PR003733, PR003899, PR006002, PR006003, PR008551) were tested for cytotoxicity on MDA-MB-468, and the results are shown in FIG. 11 and Table 10.

TABLE 10
Characteristic parameters of cytotoxicity to MDA-MB-468.

| | | | | | Max |
|---|---|---|---|---|---|
| | | | | EC50 | cytotoxicity |
| Format | B7H4×CD3 | Anti-B7H4 | Anti-CD3 | (nM) | (%) |
| Fab-Fc-scFv | PR003733 | PR003366 | PR001848 (SP34) | 0.02484 | 95.16 |
| Fab-Fc-scFv | PR003899 | PR003366 | PR003886 (SP34) | 0.1124 | 96.31 |
| VH-Fc-scFv | PR006002 | PR003366 | PR006364 (A1) | 0.03301 | 94.43 |
| VH-Fc-scFv | PR006003 | PR003366 | PR006365 (E2) | 0.04652 | 95.06 |
| VH-Fc-scFv | PR008551 | PR003366 | PR010991 (3G11) | 15.18 | 90.7 |

As shown in (A) of FIG. 11, A1 and E2 derived B7H4xCD3 bispecific antibodies PR006002 and PR006003 can raise strong and dose dependent cytotoxicity to MDA-MB-468 cells, with comparable potency to that of PR003733 which was derived from humanized SP34. As shown in (B) of FIG. 11, 3G11 derived B7H4xCD3 bispecific antibody PR008551 can also raise dose-dependent cytotoxicity to MDA-MB-468 cells with reaching to >90% of top cytotoxicity. The results confirmed the potent bioactivities of anti-CD3 VHH sequences (A1, E2, 3G11) of this invention.

### Example 10: Humanization for PR006364 (A1-HCAb)

The variable region of alpaca-derived anti-CD3 PR006364 (A1-HCAb) was further humanized by established techniques, e.g. “CDR-grafting”. Briefly, the three CDR regions of PR006364 were grafted onto the four framework regions provided from the human germline gene segments IGHV3-23 and IGHJ4 to generate humanized variant sequences, in the order of FWR1-HCDR1-FWR2-HCDR2-FWR3-HCDR3-FWR4; in which, the sequences of framework regions FWR1, FWR2 and FWR3 were derived from IGHV3-23, and the sequence of framework FWR4 was derived from IGHJ4, as defined by Chothia numbering system. Additionally, one or more key residues in framework regions were changed back to the counterpart residues at parental VH of PR006364, in order to retain the biological activity.

Totally, 5 humanized variants of PR006364 were generated: PR011628, PR011629, PR011630, PR011631 and PR011632. The amino acid sequences of PR006364-derived humanized variants and their CDR regions are listed in Table 12. The “humanness H-score” which indicates how the specific antibody sequences look like human antibodies in real world, was used to rank these humanized sequences together with their parental VH sequences from alpacas. The “humanness H-score” for each VH sequence was calculated by online tool Hscore (URL: http://www.bioinf.org.uk/abs/shab/), and listed in Table 11. The H-score values of the humanized variants are higher than that of PR006364, suggesting that the humanized variants have high homology to human antibodies.

The humanized variants were produced by the methods described in Example 4. The purified antibody samples were then characterized for binding activity to human CD3E&D heterodimer protein by the method described in Example 7, binding activity to Jurkat-NFAT-Luc reporter cells by the method described in Example 6.

TABLE 11
Summary of humanized VH sequences with “humanness H-score”
and characteristics of transient production.

| | Germline | Humanness | | SEC Purity | Yield | Conc. |
|---|---|---|---|---|---|---|
| Antibody ID | (by IMGT) | (H-score) | Sample Lot. # | (%) | (mg/L) | (mg/ml) |
| PR006364 | Alpaca | −1.296 | 20220826B003 | 97.290 | 476.7 | 6.81 |
| | IGHV3-3*01 | | | | | |
| PR006365 | Alpaca | −1.209 | 20220829B009 | 99.644 | 477.8 | 8.03 |
| | IGHV3-3*01 | | | | | |
| PR011628 | Human | 0.818 | 20221116M004 | 99.639 | 561.7 | 4.72 |
| | IGHV3-23*01 | | | | | |
| PR011629 | Human | 0.236 | 20221116M005 | 99.562 | 733.7 | 6.67 |
| | IGHV3-23*01 | | | | | |
| PR011630 | Human | 0.246 | 20221116M006 | 89.169 | 548.0 | 5.83 |
| | IGHV3-23*01 | | | | | |
| PR011631 | Human | 0.828 | 20221116M007 | 90.806 | 465.9 | 4.48 |
| | IGHV3-23*01 | | | | | |
| PR011632 | Human | 0.784 | 20221116M008 | 100.000 | 80.0 | 1.00 |
| | IGHV3-23*04 | | | | | |

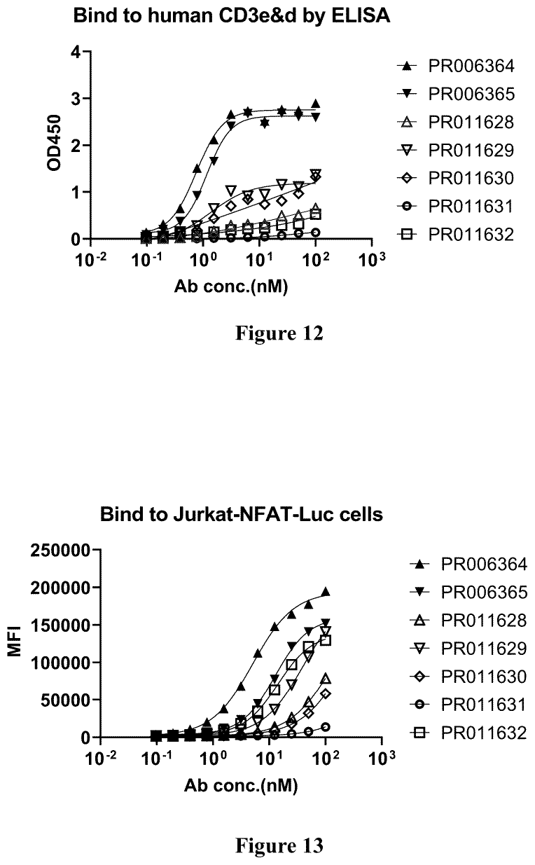

As shown in FIG. 12 and FIG. 13, all humanized variants of PR006364 can bind to human CD3E&D heterodimer protein and Jurkat-NFAT-Luc reporter cells in dose-dependent manner, though with different affinities. This result suggests that the humanization for A1-HCAb is successful.

TABLE 12
Sequences of the humanized variants of PR006364.

| SEQ ID NO: | PR011628 | anti-CD3 S135 HCAb hIgG1(C220S,AAG) |
|---|---|---|
| 145 | PR011628 Heavy Chain | EVQLLESGGGLVQPGGSLRLSCAASGRTSGIPAMSW |
| | Sequence | VRQAPGKGLEWVAMAWSGGGTIYADSVKGRFTISR |
| | | DNSKNTLYLQMNSLRAEDTAVYYCAAGRSTSNRAD |
| | | QYNYWGQGTLVTVSSASEPKSSDKTHTCPPCPAPEA |
| | | AGGPSVFLFPPKPKDTLMISRTPEVTCVVVDVSHEDP |
| | | EVKFNWYVDGVEVHNAKTKPREEQYNSTYRVVSV |
| | | LTVLHQDWLNGKEYKCKVSNKALGAPIEKTISKAK |
| | | GQPREPQVYTLPPSREEMTKNQVSLTCLVKGFYPSDI |
| | | AVEWESNGQPENNYKTTPPVLDSDGSFFLYSKLTVD |
| | | KSRWQQGNVFSCSVMHEALHNHYTQKSLSLSPGK |
| 111 | PR011628 VH Sequence | EVQLLESGGGLVQPGGSLRLSCAASGRTSGIPAMSW |
| | | VRQAPGKGLEWVAMAWSGGGTIYADSVKGRFTISR |
| | | DNSKNTLYLQMNSLRAEDTAVYYCAAGRSTSNRAD |
| | | QYNYWGQGTLVTVSS |
| 2 | PR011628 VH FWR1 | EVQLLESGGGLVQPGGSLRLSCAAS |
| | (Chothia) | |
| 17 | PR011628 VH CDR1 | GRTSGIP |
| | (Chothia) | |
| 32 | PR011628 VH FWR2 | AMSWVRQAPGKGLEWVAMA |
| | (Chothia) | |
| 41 | PR011628 VH CDR2 | WSGGG |
| | (Chothia) | |
| 54 | PR011628 VH FWR3 | TIYADSVKGRFTISRDNSKNTLYLQMNSLRAEDTAV |
| | (Chothia) | YYCAA |
| 62 | PR011628 VH CDR3 | GRSTSNRADQYNY |
| | (Chothia) | |
| 81 | PR011628 VH FWR4 | WGQGTLVTVSS |
| | (Chothia) | |
| SEQ ID NO: | PR011629 | anti-CD3 S136 HCAb hIgG1(C220S,AAG) |
| 146 | PR011629 Heavy Chain | EVQLLESGGGLVQPGGSLRLSCAASGRTSGIPAMSW |
| | Sequence | VRQAPGKEREFVAMAWSGGGTIYADSVKGRFTISR |
| | | DNSKNTLYLQMNSLRAEDTAVYYCAAGRSTSNRAD |
| | | QYNYWGQGTQVTVSSASEPKSSDKTHTCPPCPAPEA |
| | | AGGPSVFLFPPKPKDTLMISRTPEVTCVVVDVSHEDP |
| | | EVKFNWYVDGVEVHNAKTKPREEQYNSTYRVVSV |
| | | LTVLHQDWLNGKEYKCKVSNKALGAPIEKTISKAK |
| | | GQPREPQVYTLPPSREEMTKNQVSLTCLVKGFYPSDI |
| | | AVEWESNGQPENNYKTTPPVLDSDGSFFLYSKLTVD |
| | | KSRWQQGNVFSCSVMHEALHNHYTQKSLSLSPGK |
| 112 | PR011629 VH Sequence | EVQLLESGGGLVQPGGSLRLSCAASGRTSGIPAMSW |
| | | VRQAPGKEREFVAMAWSGGGTIYADSVKGRFTISR |
| | | DNSKNTLYLQMNSLRAEDTAVYYCAAGRSTSNRAD |
| | | QYNYWGQGTQVTVSS |
| 2 | PR011629 VH FWR1 | EVQLLESGGGLVQPGGSLRLSCAAS |
| | (Chothia) | |
| 17 | PR011629 VH CDR1 | GRTSGIP |
| | (Chothia) | |
| 33 | PR011629 VH FWR2 | AMSWVRQAPGKEREFVAMA |
| | (Chothia) | |
| 41 | PR011629 VH CDR2 | WSGGG |
| | (Chothia) | |
| 54 | PR011629 VH FWR3 | TIYADSVKGRFTISRDNSKNTLYLQMNSLRAEDTAV |
| | (Chothia) | YYCAA |
| 62 | PR011629 VH CDR3 | GRSTSNRADQYNY |
| | (Chothia) | |
| 82 | PR011629 VH FWR4 | WGQGTQVTVSS |
| | (Chothia) | |
| SEQ ID NO: | PR011630 | anti-CD3 S137 HCAb hIgG1(C220S,AAG) |
| 147 | PR011630 Heavy Chain | EVQLLESGGGLVQPGGSLRLSCAASGRTSGIPAMSW |
| | Sequence | VRQAPGKEREFVAMAWSGGGTIYADSVKGRFTISR |
| | | DNSKNTLYLQMNSLRAEDTAVYYCAAGRSTSNYAD |
| | | QYNYWGQGTQVTVSSASEPKSSDKTHTCPPCPAPEA |
| | | AGGPSVFLFPPKPKDTLMISRTPEVTCVVVDVSHEDP |
| | | EVKFNWYVDGVEVHNAKTKPREEQYNSTYRVVSV |
| | | LTVLHQDWLNGKEYKCKVSNKALGAPIEKTISKAK |
| | | GQPREPQVYTLPPSREEMTKNQVSLTCLVKGFYPSDI |
| | | AVEWESNGQPENNYKTTPPVLDSDGSFFLYSKLTVD |
| | | KSRWQQGNVFSCSVMHEALHNHYTQKSLSLSPGK |
| 113 | PR011630 VH Sequence | EVQLLESGGGLVQPGGSLRLSCAASGRTSGIPAMSW |
| | | VRQAPGKEREFVAMAWSGGGTIYADSVKGRFTISR |
| | | DNSKNTLYLQMNSLRAEDTAVYYCAAGRSTSNYAD |
| | | QYNYWGQGTQVTVSS |
| 2 | PR011630 VH FWR1 | EVQLLESGGGLVQPGGSLRLSCAAS |
| | (Chothia) | |
| 17 | PR011630 VH CDR1 | GRTSGIP |
| | (Chothia) | |
| 33 | PR011630 VH FWR2 | AMSWVRQAPGKEREFVAMA |
| | (Chothia) | |
| 41 | PR011630 VH CDR2 | WSGGG |
| | (Chothia) | |
| 54 | PR011630 VH FWR3 | TIYADSVKGRFTISRDNSKNTLYLQMNSLRAEDTAV |
| | (Chothia) | YYCAA |
| 65 | PR011630 VH CDR3 | GRSTSNYADQYNY |
| | (Chothia) | |
| 82 | PR011630 VH FWR4 | WGQGTQVTVSS |
| | (Chothia) | |
| SEQ ID NO: | PR011631 | anti-CD3 S138 HCAb hIgG1(C220S,AAG) |
| 148 | PR011631 Heavy Chain | EVQLLESGGGLVQPGGSLRLSCAASGRTSGIPAMSW |
| | Sequence | VRQAPGKGLEWVAMAWSGGGTIYADSVKGRFTISR |
| | | DNSKNTLYLQMNSLRAEDTAVYYCAAGRSTSNYAD |
| | | QYNYWGQGTLVTVSSASEPKSSDKTHTCPPCPAPEA |
| | | AGGPSVFLFPPKPKDTLMISRTPEVTCVVVDVSHEDP |
| | | EVKFNWYVDGVEVHNAKTKPREEQYNSTYRVVSV |
| | | LTVLHQDWLNGKEYKCKVSNKALGAPIEKTISKAK |
| | | GQPREPQVYTLPPSREEMTKNQVSLTCLVKGFYPSDI |
| | | AVEWESNGQPENNYKTTPPVLDSDGSFFLYSKLTVD |
| | | KSRWQQGNVFSCSVMHEALHNHYTQKSLSLSPGK |
| 114 | PR011631 VH Sequence | EVQLLESGGGLVQPGGSLRLSCAASGRTSGIPAMSW |
| | | VRQAPGKGLEWVAMAWSGGGTIYADSVKGRFTISR |
| | | DNSKNTLYLQMNSLRAEDTAVYYCAAGRSTSNYAD |
| | | QYNYWGQGTLVTVSS |
| 2 | PR011631 VH FWR1 | EVQLLESGGGLVQPGGSLRLSCAAS |
| | (Chothia) | |
| 17 | PR011631 VH CDR1 | GRTSGIP |
| | (Chothia) | |
| 32 | PR011631 VH FWR2 | AMSWVRQAPGKGLEWVAMA |
| | (Chothia) | |
| 41 | PR011631 VH CDR2 | WSGGG |
| | (Chothia) | |
| 54 | PR011631 VH FWR3 | TIYADSVKGRFTISRDNSKNTLYLQMNSLRAEDTAV |
| | (Chothia) | YYCAA |
| 65 | PR011631 VH CDR3 | GRSTSNYADQYNY |
| | (Chothia) | |
| 81 | PR011631 VH FWR4 | WGQGTLVTVSS |
| | (Chothia) | |
| SEQ ID NO: | PR011632 | anti-CD3 S140 HCAb hIgG1(C220S,AAG) |
| 149 | PR011632 Heavy Chain | EVQLVESGGGLVQPGGSLRLSCAASGRTSGIPAMAW |
| | Sequence | FRQAPGKGLEWVAMAWSGGGTIYADSVKGRFTISR |
| | | DNSKNTLYLQMNSLRAEDTAVYYCARGRSTSNRAD |
| | | QYNYWGQGTLVTVSSASEPKSSDKTHTCPPCPAPEA |
| | | AGGPSVFLFPPKPKDTLMISRTPEVTCVVVDVSHEDP |
| | | EVKFNWYVDGVEVHNAKTKPREEQYNSTYRVVSV |
| | | LTVLHQDWLNGKEYKCKVSNKALGAPIEKTISKAK |
| | | GQPREPQVYTLPPSREEMTKNQVSLTCLVKGFYPSDI |
| | | AVEWESNGQPENNYKTTPPVLDSDGSFFLYSKLTVD |
| | | KSRWQQGNVFSCSVMHEALHNHYTQKSLSLSPGK |
| 115 | PR011632 VH Sequence | EVQLVESGGGLVQPGGSLRLSCAASGRTSGIPAMAW |
| | | FRQAPGKGLEWVAMAWSGGGTIYADSVKGRFTISR |
| | | DNSKNTLYLQMNSLRAEDTAVYYCARGRSTSNRAD |
| | | QYNYWGQGTLVTVSS |
| 11 | PR011632 VH FWR1 | EVQLVESGGGLVQPGGSLRLSCAAS |
| | (Chothia) | |
| 17 | PR011632 VH CDR1 | GRTSGIP |
| | (Chothia) | |
| 34 | PR011632 VH FWR2 | AMAWFRQAPGKGLEWVAMA |
| | (Chothia) | |
| 41 | PR011632 VH CDR2 | WSGGG |
| | (Chothia) | |
| 55 | PR011632 VH FWR3 | TIYADSVKGRFTISRDNSKNTLYLQMNSLRAEDTAV |
| | (Chothia) | YYCAR |
| 62 | PR011632 VH CDR3 | GRSTSNRADQYNY |
| | (Chothia) | |
| 81 | PR011632 VH FWR4 | WGQGTLVTVSS |
| | (Chothia) | |

### Example 11: B7H4xCD3 Bispecific Antibodies Derived from Humanized PR006364

In order to evaluate if the humanized variants of anti-CD3 PR006364 (A1-HCAb) can bring T cell mediated cytotoxicity, multiple B7H4xCD3 bispecific antibodies (listed in Table 13) were generated in the format of ‘VH-Fc-scFv’ as illustrated in FIG. 10, by composing one B7H4-binding portion from anti-B7H4 PR003366 and one CD3-binding portion from the humanized variants. As described previously in Example 8, “knob-into-hole” technology was employed to enhance the formation of heterodimeric heavy chains, and mutations “AAA” were used to remove Fc effector functions. The sequences of polypeptide chains of each B7H4xCD3 are listed in Table 14.

The recombinant B7H4xCD3 bispecific antibodies were produced by the method described in Example 8. The expression yield and purity determined by SEC-HPLC are listed in Table 13. All the B7H4xCD3 samples showed good productivity.

TABLE 13
Antibody production summary for B7H4×CD3
derived from humanized PR006364.

| | | | | SEC | |
|---|---|---|---|---|---|
| | | Anti-CD3 | Sample | Purity | Yield |
| B7H4×CD3 | Anti-B7H4 | HCAb | Lot. # | (%) | (mg/L) |
| PR006002 | PR003366 | PR006364 | 20201030A13 | 100 | 80 |
| PR010982 | PR003366 | PR011628 | CY20221008 | 96.66 | 78 |
| PR010983 | PR003366 | PR011629 | CY20221008 | 96.06 | 94 |
| PR010984 | PR003366 | PR011630 | CY20221008 | 95.98 | 111 |
| PR010985 | PR003366 | PR011631 | CY20221008 | 95.42 | 89 |
| PR010987 | PR003366 | PR011632 | CY20221008 | 98.37 | 185 |

TABLE 14
Polypeptide chain sequences of B7H4xCD3 bispecific antibodies derived from
humanized PR006364.

| | | anti-B7H4xCD3 VH(A1)-Fc-scFv(PR003366), hIgG1(dCH1, |
|---|---|---|
| SEQ ID NO: | PR006002 | KIH, LALA) |
| 175 | anti-CD3 VH-Fc | RIRLVESGGGLVQAGGSLRLSCAASGRTSGIPAMAWFRQ |
| | chain | APGKEREFVAMAWSGGGTIYGDSVKNRFTISRDNTQNTI |
| | | FLQMNSLKPEDTALYYCAAGRSTSNRADQYNYWGQGT |
| | | QVTVSSASEPKSSDKTHTCPPCPAPEAAGGPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPCREEMTKNQ |
| | | VSLWCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| 170 | anti-B7H4 scFv-Fc | EIVMTQSPASLSVSPGERATLSCRASQSVSSNLAWYQQK |
| | chain | PGQAPRLLIYGASTRATGIPARVSGGGSGTEFTLTISSLQS |
| | | EDFAVYYCQQYKNWPFTFGPGTKLEIKGGGGSGGGGSG |
| | | GGGSGGGGSQVQLVQSGAEVKKPGSSMRVSCKASEDTF |
| | | SSYAISWVRQAPGQGLEWMGGTAPIFGTTNYAQKFQGR |
| | | VTITADKSTSTAYMELSSLRSEDTAVYYCARGGPYFDYW |
| | | GQGTLVTVSSASEPKSSDKTHTCPPCPAPEAAGGPSVFLF |
| | | PPKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVCTLPPSREEM |
| | | TKNQVSLSCAVKGFYPSDIAVEWESNGQPENNYKTTPPV |
| | | LDSDGSFFLVSKLTVDKSRWQQGNVFSCSVMHEALHNH |
| | | YTQKSLSLSPGK |
| | | anti-B7H4xCD3 S135-Fc-scFv(PR003366), hIgG1(dCH1, |
| SEQ ID NO: | PR010982 | KIH, AAA) |
| 179 | anti-CD3 VH-Fc | EVQLLESGGGLVQPGGSLRLSCAASGRTSGIPAMSWVRQ |
| | chain | APGKGLEWVAMAWSGGGTIYADSVKGRFTISRDNSKNT |
| | | LYLQMNSLRAEDTAVYYCAAGRSTSNRADQYNYWGQG |
| | | TLVTVSSASEPKSSDKTHTCPPCPAPEAAGAPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPCREEMTKNQ |
| | | VSLWCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| 178 | anti-B7H4 scFv-Fc | EIVMTQSPASLSVSPGERATLSCRASQSVSSNLAWYQQK |
| | chain | PGQAPRLLIYGASTRATGIPARVSGGGSGTEFTLTISSLQS |
| | | EDFAVYYCQQYKNWPFTFGPGTKLEIKGGGGSGGGGSG |
| | | GGGSGGGGSQVQLVQSGAEVKKPGSSMRVSCKASEDTF |
| | | SSYAISWVRQAPGOGLEWMGGTAPIFGTTNYAQKFQGR |
| | | VTITADKSTSTAYMELSSLRSEDTAVYYCARGGPYFDYW |
| | | GQGTLVTVSSASEPKSSDKTHTCPPCPAPEAAGAPSVFLF |
| | | PPKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVCTLPPSREEM |
| | | TKNQVSLSCAVKGFYPSDIAVEWESNGQPENNYKTTPPV |
| | | LDSDGSFFLVSKLTVDKSRWQQGNVFSCSVMHEALHNH |
| | | YTQKSLSLSPGK |
| | | anti-B7H4xCD3 S136-Fc-scFv(PR003366), hIgG1(dCH1, |
| SEQ ID NO: | PR010983 | KIH, AAA) |
| 180 | anti-CD3 VH-Fc | EVQLLESGGGLVQPGGSLRLSCAASGRTSGIPAMSWVRQ |
| | chain | APGKEREFVAMAWSGGGTIYADSVKGRFTISRDNSKNTL |
| | | YLQMNSLRAEDTAVYYCAAGRSTSNRADQYNYWGQGT |
| | | QVTVSSASEPKSSDKTHTCPPCPAPEAAGAPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPCREEMTKNQ |
| | | VSLWCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| 178 | anti-B7H4 scFv-Fc | EIVMTQSPASLSVSPGERATLSCRASQSVSSNLAWYQQK |
| | chain | PGQAPRLLIYGASTRATGIPARVSGGGSGTEFTLTISSLQS |
| | | EDFAVYYCQQYKNWPFTFGPGTKLEIKGGGGSGGGGSG |
| | | GGGSGGGGSQVQLVQSGAEVKKPGSSMRVSCKASEDTF |
| | | SSYAISWVRQAPGQGLEWMGGTAPIFGTTNYAQKFQGR |
| | | VTITADKSTSTAYMELSSLRSEDTAVYYCARGGPYFDYW |
| | | GQGTLVTVSSASEPKSSDKTHTCPPCPAPEAAGAPSVFLF |
| | | PPKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVCTLPPSREEM |
| | | TKNQVSLSCAVKGFYPSDIAVEWESNGQPENNYKTTPPV |
| | | LDSDGSFFLVSKLTVDKSRWQQGNVFSCSVMHEALHNH |
| | | YTQKSLSLSPGK |
| | | anti-B7H4xCD3 S137-Fc-scFv(PR003366), hIgG1(dCH1, |
| SEQ ID NO: | PR010984 | KIH, AAA) |
| 181 | anti-CD3 VH-Fc | EVQLLESGGGLVQPGGSLRLSCAASGRTSGIPAMSWVRQ |
| | chain | APGKEREFVAMAWSGGGTIYADSVKGRFTISRDNSKNTL |
| | | YLQMNSLRAEDTAVYYCAAGRSTSNYADQYNYWGQGT |
| | | QVTVSSASEPKSSDKTHTCPPCPAPEAAGAPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPCREEMTKNQ |
| | | VSLWCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| 178 | anti-B7H4 scFv-Fc | EIVMTQSPASLSVSPGERATLSCRASQSVSSNLAWYQQK |
| | chain | PGQAPRLLIYGASTRATGIPARVSGGGSGTEFTLTISSLQS |
| | | EDFAVYYCQQYKNWPFTFGPGTKLEIKGGGGSGGGGSG |
| | | GGGSGGGGSQVQLVQSGAEVKKPGSSMRVSCKASEDTF |
| | | SSYAISWVRQAPGQGLEWMGGTAPIFGTTNYAQKFQGR |
| | | VTITADKSTSTAYMELSSLRSEDTAVYYCARGGPYFDYW |
| | | GQGTLVTVSSASEPKSSDKTHTCPPCPAPEAAGAPSVFLF |
| | | PPKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVCTLPPSREEM |
| | | TKNQVSLSCAVKGFYPSDIAVEWESNGQPENNYKTTPPV |
| | | LDSDGSFFLVSKLTVDKSRWQQGNVFSCSVMHEALHNH |
| | | YTQKSLSLSPGK |
| | | anti-B7H4xCD3 S138-Fc-scFv(PR003366), hIgG1(dCH1, |
| SEQ ID NO: | PR010985 | KIH, AAA) |
| 182 | anti-CD3 VH-Fc | EVQLLESGGGLVQPGGSLRLSCAASGRTSGIPAMSWVRQ |
| | chain | APGKGLEWVAMAWSGGGTIYADSVKGRFTISRDNSKNT |
| | | LYLQMNSLRAEDTAVYYCAAGRSTSNYADQYNYWGQG |
| | | TLVTVSSASEPKSSDKTHTCPPCPAPEAAGAPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPCREEMTKNQ |
| | | VSLWCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| 178 | anti-B7H4 scFv-Fc | EIVMTQSPASLSVSPGERATLSCRASQSVSSNLAWYQQK |
| | chain | PGQAPRLLIYGASTRATGIPARVSGGGSGTEFTLTISSLQS |
| | | EDFAVYYCQQYKNWPFTFGPGTKLEIKGGGGSGGGGSG |
| | | GGGSGGGGSQVQLVQSGAEVKKPGSSMRVSCKASEDTF |
| | | SSYAISWVRQAPGQGLEWMGGTAPIFGTTNYAQKFQGR |
| | | VTITADKSTSTAYMELSSLRSEDTAVYYCARGGPYFDYW |
| | | GQGTLVTVSSASEPKSSDKTHTCPPCPAPEAAGAPSVFLF |
| | | PPKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVCTLPPSREEM |
| | | TKNQVSLSCAVKGFYPSDIAVEWESNGQPENNYKTTPPV |
| | | LDSDGSFFLVSKLTVDKSRWQQGNVFSCSVMHEALHNH |
| | | YTQKSLSLSPGK |
| | | anti-B7H4xCD3 S140-Fc-scFv(PR003366), hIgG1(dCH1, |
| SEQ ID NO: | PR010987 | KIH, AAA) |
| 183 | anti-CD3 VH-Fc | EVQLVESGGGLVQPGGSLRLSCAASGRTSGIPAMAWFRQ |
| | chain | APGKGLEWVAMAWSGGGTIYADSVKGRFTISRDNSKNT |
| | | LYLQMNSLRAEDTAVYYCARGRSTSNRADQYNYWGQG |
| | | TLVTVSSASEPKSSDKTHTCPPCPAPEAAGAPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPCREEMTKNQ |
| | | VSLWCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| 178 | anti-B7H4 scFv-Fc | EIVMTQSPASLSVSPGERATLSCRASQSVSSNLAWYQQK |
| | chain | PGQAPRLLIYGASTRATGIPARVSGGGSGTEFTLTISSLQS |
| | | EDFAVYYCQQYKNWPFTFGPGTKLEIKGGGGSGGGGSG |
| | | GGGSGGGGSQVQLVQSGAEVKKPGSSMRVSCKASEDTF |
| | | SSYAISWVRQAPGQGLEWMGGTAPIFGTTNYAQKFQGR |
| | | VTITADKSTSTAYMELSSLRSEDTAVYYCARGGPYFDYW |
| | | GQGTLVTVSSASEPKSSDKTHTCPPCPAPEAAGAPSVFLF |
| | | PPKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVCTLPPSREEM |
| | | TKNQVSLSCAVKGFYPSDIAVEWESNGQPENNYKTTPPV |
| | | LDSDGSFFLVSKLTVDKSRWQQGNVFSCSVMHEALHNH |
| | | YTQKSLSLSPGK |

The T cell mediated cytotoxicity of B7H4xCD3 bispecific antibodies to specific target cells were determined by RTCA method as described in Example 9. In this example, 1B71H4 highly expressed tumor cell MDA-MB-468 (ATCC, HTB-132) and 1B71H4-negative tumor cell MDA-MB-231 (ATCC, HTB-26) were used as target cells, and isolated human T cells were used as effector cells, the E:T ratio was 10:1. The cells were incubated with testing antibody samples for 24 hours before analysis.

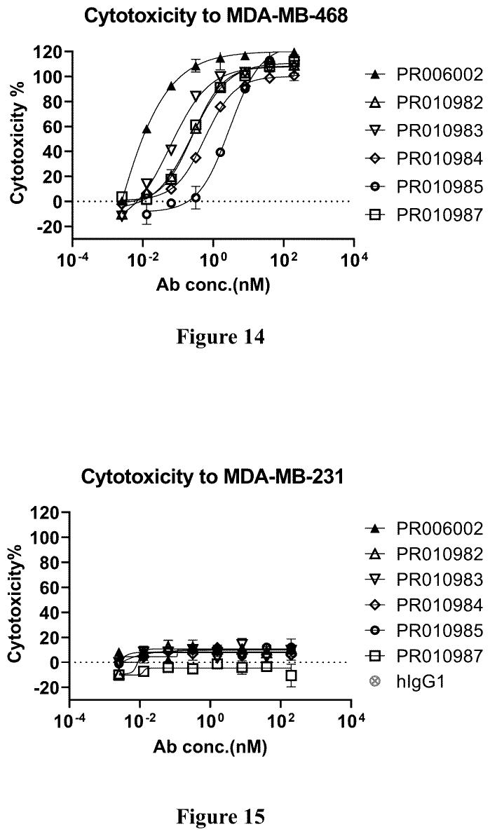

As shown in FIG. 14 and Table 15, all of B7H4xCD3 bispecific antibodies generated from PR006364 and humanized variants can raise strong and dose dependent cytotoxicity to MDA-MB-468 cells. And the maximum cytotoxicity can reach to almost 100%, suggesting the complete lysis for B7H4 target cells; although the EC50 values were different, which was in alignment with different binding activities to CD3 as described previously (FIG. 13). On the other hand, as shown in FIG. 15, none of them can bring obvious cytotoxicity to MDA-MB-231 cells, which suggests that the cytotoxicity of B7H4xCD3 is specific to B7H4-positive cells. Furthermore, the different cytotoxicity potency of these humanized variants with almost same maximum cytotoxicity but various EC50 values will give multiple options for generating T cell engager bispecific antibodies in different therapeutic scenarios.

TABLE 15
Characteristic parameters of cytotoxicity to MDA-MB-468.

| | | | | Max |
|---|---|---|---|---|
| | | | EC50 | cytotoxicity |
| B7H4×CD3 | Anti-B7H4 | Anti-CD3 | (nM) | (%) |
| PR006002 | PR003366 | PR006364 | 0.001114 | 120.2 |
| PR010982 | PR003366 | PR011628 | 0.2471 | 111 |
| PR010983 | PR003366 | PR011629 | 0.06015 | 108 |
| PR010984 | PR003366 | PR011630 | 0.5758 | 100.2 |
| PR010985 | PR003366 | PR011631 | 2.91 | 125.1 |
| PR010987 | PR003366 | PR011632 | 0.2628 | 108.5 |

### Example 12: Evaluate Cross Reactivity to Cynomolgus Monkey

The cross reactivity to cynomolgus monkey for 3G11-HCAb (PR010991) and derived B7H4xCD3 PR008551 was evaluated by assays of binding activity to cynomolgus monkey PBMCs by FACS method and T cell mediated cytotoxicity with cynomolgus monkey PBMCs by RTCA method. In this example, Anti-CD3/TCR HCAb PR006361 derived from patent WO2016180982A1 and its derived B7H4xCD3 PR006001 were used as control antibodies. These antibodies are generated as described in Example 8.

Cynomolgus monkey PBMCs were isolated from 10 mL fresh cynomolgus monkey blood using Ficoll Paque Plus (Cytiva 17-1440-02) following the vendor's instructions. The recombinant antibodies were diluted from 200 nM to 11 different concentrations by 2-fold serial dilution. The diluted samples were incubated with cynomolgus monkey PBMCs for 1 hour, and then the secondary antibody (Alexa Fluor®488 AffinityPure Goat Anti-Human IgG (Fcγ Fragment Specific), Jackson ImmunoResearch, 109-545-098) was added and incubated for 1 hour. The cells were washed twice with staining buffer and subjected to flow cytometry. The data were processed by FlowJo v10 (FlowJo, LLC) and non-linear curve fitting analyzed by GraphPad Prism 8.

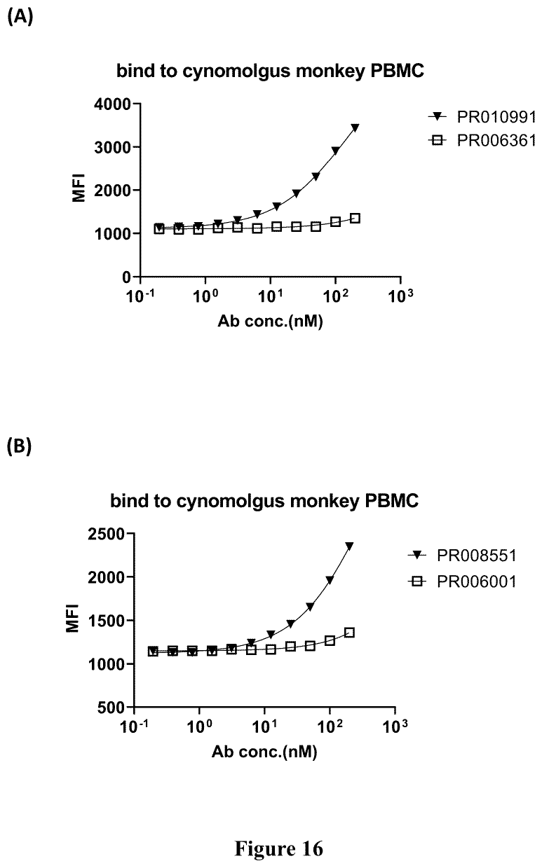

As showed in FIG. 16, 3G11-HCAb (PR010991) and derived B7H4xCD3 PR008551 can bind to cynomolgus monkey PBMCs in dose-dependent manner, but control antibody PR006361 and derived B7H4xCD3 PR006001 can not. This suggests 3G11 has cross reactivity to cynomolgus monkey, which shows advantages over PR006361 from prior art.

The T cell mediated cytotoxicity with cynomolgus monkey PBMCs for B7H4xCD3 PR008551 was determined by RTCA method as described in Example 9. In this example, B7H4 highly expressed MDA-MB-468 cells were used as target cells, and cynomolgus monkey PBMCs were used as effector cells, the E:T ratio was 10:1. The test antibody was 5-fold serial diluted from 100 nM to 8 concentrations. The cells were incubated with testing antibody samples for 24 hours or 48 hours before analysis.

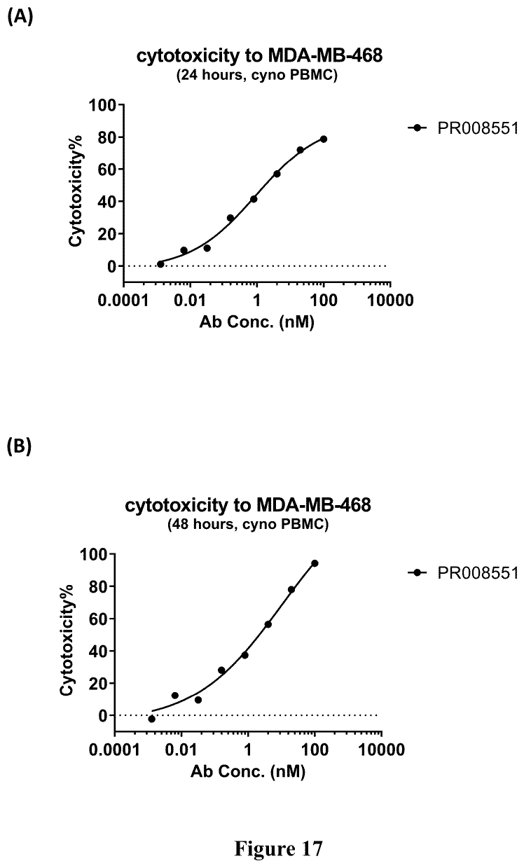

As shown in FIG. 17, PR008551 induced dose-dependent cynomolgus monkey PBMC mediated cytotoxicity to MDA-MB-468 cells; the top cytotoxicity reached to almost 80% of the maximum after 24 hours incubation and increased to higher than 95% after 48 hours incubation indicating almost complete lysis to the target cells.

Combining the results of FIG. 16 and FIG. 17, it approves 3G11-HCAb (PR010991) has cross reactivity to cynomolgus monkey; it can bind and activate CD3 on T cells in cynomolgus monkey PBMCs and bring cytotoxicity to tumor cells in the context of CD3 engager bispecific antibodies.

### Example 13: 3G11-HCAb derived variants

In order to identify what residues within VH region of 3G11-HCAb (PR010991) are contributing to the binding activity to CD3, multiple VH variants were derived from 3G11 by introducing mutations into CDRs by saturation mutagenesis or other techniques.

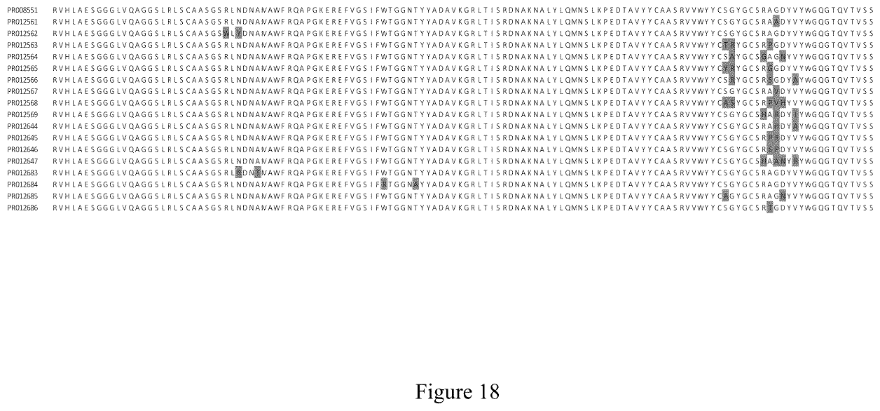

Totally 17 VH variants were obtained. These VH variants were constructed into the vector pTT5-VHH-hFc to generate recombinant HCAbs of PR010991's derivatives, and also constructed into the plasmids encoding polypeptide chains of PR008551 to generate recombinant B7H4xCD3 of PR008551's derivatives. The newly generated HCAb antibodies and B7H4xCD3 bispecific antibodies are listed in Table 16 and their amino acid sequences are listed in Table 17 and Table 18. The alignment for VH sequences of 3G11 VH (as part of PR008551) and its variants from PR008551's derivatives is shown in FIG. 18.

TABLE 16
3G11-HCAb derived variants and generated
B7H4×CD3 bispecific antibodies.

| 3G11 derived variant | Ab ID for HCAb | Ab ID for B7H4×CD3 |
|---|---|---|
| 3G11 | PR010991 | PR008551 |
| 3G11_S1 | PR012876 | PR012561 |
| 3G11_S2 | PR012877 | PR012562 |
| 3G11_S3 | PR012878 | PR012563 |
| 3G11_S4 | PR012879 | PR012564 |
| 3G11_S5 | PR012880 | PR012565 |
| 3G11_S6 | PR012881 | PR012566 |
| 3G11_S7 | PR012882 | PR012567 |
| 3G11_S8 | PR012883 | PR012568 |
| 3G11_S9 | PR012884 | PR012569 |
| 3G11_S10 | PR012885 | PR012644 |
| 3G11_S11 | PR012886 | PR012645 |
| 3G11_S12 | PR012887 | PR012646 |
| 3G11_S13 | PR012888 | PR012647 |
| 3G11_S14 | PR012889 | PR012683 |
| 3G11_S15 | PR012890 | PR012684 |
| 3G11_S16 | PR012891 | PR012685 |
| 3G11_S17 | PR012892 | PR012686 |

TABLE 17
Sequences of 3G11-HCAb derived HCAb variants.

| SEQ ID NO: | PR012876 | anti-CD3 3G11_S1 HCAb hIgG1(C220S, AAA) |
|---|---|---|
| 150 | PR012876 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSRAADYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 116 | PR012876 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSRAADYVYWGQGTQVTVSS |
| 10 | PR012876 VH FWR1 | RVHLAESGGGLVQAGGSLRLSCAAS |
| | (Chothia) | |
| 20 | PR012876 VH CDR1 | GSRLNDN |
| | (Chothia) | |
| 31 | PR012876 VH FWR2 | AMAWFRQAPGKEREFVGSI |
| | (Chothia) | |
| 42 | PR012876 VH CDR2 | FWTGGN |
| | (Chothia) | |
| 53 | PR012876 VH FWR3 | TYYADAVKGRLTISRDNAKNALYLQMNSLKPEDTAV |
| | (Chothia) | YYCAA |
| 66 | PR012876 VH CDR3 | SRVVWYYCSGYGCSRAADYVY |
| | (Chothia) | |
| 82 | PR012876 VH FWR4 | WGQGTQVTVSS |
| | (Chothia) | |
| SEQ ID NO: | PR012877 | anti-CD3 3G11_S2 HCAb hIgG1(C220S, AAA) |
| 151 | PR012877 Heavy Chain | RVHLAESGGGLVQAGGSLRLSCAASGSWLYDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSRAGDYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 117 | PR012877 VH Sequence | RVHLAESGGGLVQAGGSLRLSCAASGSWLYDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSRAGDYVYWGQGTQVTVSS |
| 10 | PR012877 VH FWR1 | RVHLAESGGGLVQAGGSLRLSCAAS |
| | (Chothia) | |
| 21 | PR012877 VH CDR1 | GSWLYDN |
| | (Chothia) | |
| 31 | PR012877 VH FWR2 | AMAWFRQAPGKEREFVGSI |
| | (Chothia) | |
| 42 | PR012877 VH CDR2 | FWTGGN |
| | (Chothia) | |
| 53 | PR012877 VH FWR3 | TYYADAVKGRLTISRDNAKNALYLQMNSLKPEDTAV |
| | (Chothia) | YYCAA |
| 64 | PR012877 VH CDR3 | SRVVWYYCSGYGCSRAGDYVY |
| | (Chothia) | |
| 82 | PR012877 VH FWR4 | WGQGTQVTVSS |
| | (Chothia) | |
| SEQ ID NO: | PR012878 | anti-CD3 3G11_S3 HCAb hIgG1(C220S, AAA) |
| 152 | PR012878 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCT |
| | | RYGCSRPGDYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 118 | PR012878 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCT |
| | | RYGCSRPGDYVYWGQGTQVTVSS |
| 10 | PR012878 VH FWR1 | RVHLAESGGGLVQAGGSLRLSCAAS |
| | (Chothia) | |
| 20 | PR012878 VH CDR1 | GSRLNDN |
| | (Chothia) | |
| 31 | PR012878 VH FWR2 | AMAWFRQAPGKEREFVGSI |
| | (Chothia) | |
| 42 | PR012878 VH CDR2 | FWTGGN |
| | (Chothia) | |
| 53 | PR012878 VH FWR3 | TYYADAVKGRLTISRDNAKNALYLQMNSLKPEDTAV |
| | (Chothia) | YYCAA |
| 67 | PR012878 VH CDR3 | SRVVWYYCTRYGCSRPGDYVY |
| | (Chothia) | |
| 82 | PR012878 VH FWR4 | WGQGTQVTVSS |
| | (Chothia) | |
| SEQ ID NO: | PR012879 | anti-CD3 3G11_S4 HCAb hIgG1(C220S, AAA) |
| 153 | PR012879 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | AYGCSGAGNYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 119 | PR012879 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | AYGCSGAGNYVYWGQGTQVTVSS |
| 10 | PR012879 VH FWR1 | RVHLAESGGGLVQAGGSLRLSCAAS |
| | (Chothia) | |
| 20 | PR012879 VH CDR1 | GSRLNDN |
| | (Chothia) | |
| 31 | PR012879 VH FWR2 | AMAWFRQAPGKEREFVGSI |
| | (Chothia) | |
| 42 | PR012879 VH CDR2 | FWTGGN |
| | (Chothia) | |
| 53 | PR012879 VH FWR3 | TYYADAVKGRLTISRDNAKNALYLQMNSLKPEDTAV |
| | (Chothia) | YYCAA |
| 68 | PR012879 VH CDR3 | SRVVWYYCSAYGCSGAGNYVY |
| | (Chothia) | |
| 82 | PR012879 VH FWR4 | WGQGTQVTVSS |
| | (Chothia) | |
| SEQ ID NO: | PR012880 | anti-CD3 3G11_S5 HCAb hIgG1(C220S, AAA) |
| 154 | PR012880 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCY |
| | | RYGCSRGGDYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 120 | PR012880 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCY |
| | | RYGCSRGGDYVYWGQGTQVTVSS |
| 10 | PR012880 VH FWR1 | RVHLAESGGGLVQAGGSLRLSCAAS |
| | (Chothia) | |
| 20 | PR012880 VH CDR1 | GSRLNDN |
| | (Chothia) | |
| 31 | PR012880 VH FWR2 | AMAWFRQAPGKEREFVGSI |
| | (Chothia) | |
| 42 | PR012880 VH CDR2 | FWTGGN |
| | (Chothia) | |
| 53 | PR012880 VH FWR3 | TYYADAVKGRLTISRDNAKNALYLQMNSLKPEDTAV |
| | (Chothia) | YYCAA |
| 69 | PR012880 VH CDR3 | SRVVWYYCYRYGCSRGGDYVY |
| | (Chothia) | |
| 82 | PR012880 VH FWR4 | WGQGTQVTVSS |
| | (Chothia) | |
| SEQ ID NO: | PR012881 | anti-CD3 3G11_S6 HCAb hIgG1(C220S, AAA) |
| 155 | PR012881 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | RYGCSRSGDYAYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 121 | PR012881 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | RYGCSRSGDYAYWGQGTQVTVSS |
| 10 | PR012881 VH FWR1 | RVHLAESGGGLVQAGGSLRLSCAAS |
| | (Chothia) | |
| 20 | PR012881 VH CDR1 | GSRLNDN |
| | (Chothia) | |
| 31 | PR012881 VH FWR2 | AMAWFRQAPGKEREFVGSI |
| | (Chothia) | |
| 42 | PR012881 VH CDR2 | FWTGGN |
| | (Chothia) | |
| 53 | PR012881 VH FWR3 | TYYADAVKGRLTISRDNAKNALYLQMNSLKPEDTAV |
| | (Chothia) | YYCAA |
| 70 | PR012881 VH CDR3 | SRVVWYYCSRYGCSRSGDYAY |
| | (Chothia) | |
| 82 | PR012881 VH FWR4 | WGQGTQVTVSS |
| | (Chothia) | |
| SEQ ID NO: | PR012882 | anti-CD3 3G11_S7 HCAb hIgG1(C220S, AAA) |
| 156 | PR012882 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSRAVDYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 122 | PR012882 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSRAVDYVYWGQGTQVTVSS |
| 10 | PR012882 VH FWR1 | RVHLAESGGGLVQAGGSLRLSCAAS |
| | (Chothia) | |
| 20 | PR012882 VH CDR1 | GSRLNDN |
| | (Chothia) | |
| 31 | PR012882 VH FWR2 | AMAWFRQAPGKEREFVGSI |
| | (Chothia) | |
| 42 | PR012882 VH CDR2 | FWTGGN |
| | (Chothia) | |
| 53 | PR012882 VH FWR3 | TYYADAVKGRLTISRDNAKNALYLQMNSLKPEDTAV |
| | (Chothia) | YYCAA |
| 71 | PR012882 VH CDR3 | SRVVWYYCSGYGCSRAVDYVY |
| | (Chothia) | |
| 82 | PR012882 VH FWR4 | WGQGTQVTVSS |
| | (Chothia) | |
| SEQ ID NO: | PR012883 | anti-CD3 3G11_S8 HCAb hIgG1(C220S, AAA) |
| 157 | PR012883 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCA |
| | | SYGCSRPVHYVYWGQGTQVTVSSASEPKSSDKTHTCP |
| | | PCPAPEAAGAPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| 123 | PR012883 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCA |
| | | SYGCSRPVHYVYWGQGTQVTVSS |
| 10 | PR012883 VH FWR1 | RVHLAESGGGLVQAGGSLRLSCAAS |
| | (Chothia) | |
| 20 | PR012883 VH CDR1 | GSRLNDN |
| | (Chothia) | |
| 31 | PR012883 VH FWR2 | AMAWFRQAPGKEREFVGSI |
| | (Chothia) | |
| 42 | PR012883 VH CDR2 | FWTGGN |
| | (Chothia) | |
| 53 | PR012883 VH FWR3 | TYYADAVKGRLTISRDNAKNALYLQMNSLKPEDTAV |
| | (Chothia) | YYCAA |
| 72 | PR012883 VH CDR3 | SRVVWYYCASYGCSRPVHYVY |
| | (Chothia) | |
| 82 | PR012883 VH FWR4 | WGQGTQVTVSS |
| | (Chothia) | |
| SEQ ID NO: | PR012884 | anti-CD3 3G11_S9 HCAb hIgG1(C220S, AAA) |
| 158 | PR012884 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSHARDYIYWGQGTQVTVSSASEPKSSDKTHTCP |
| | | PCPAPEAAGAPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| 124 | PR012884 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSHARDYIYWGQGTQVTVSS |
| 10 | PR012884 VH FWR1 | RVHLAESGGGLVQAGGSLRLSCAAS |
| | (Chothia) | |
| 20 | PR012884 VH CDR1 | GSRLNDN |
| | (Chothia) | |
| 31 | PR012884 VH FWR2 | AMAWFRQAPGKEREFVGSI |
| | (Chothia) | |
| 42 | PR012884 VH CDR2 | FWTGGN |
| | (Chothia) | |
| 53 | PR012884 VH FWR3 | TYYADAVKGRLTISRDNAKNALYLQMNSLKPEDTAV |
| | (Chothia) | YYCAA |
| 73 | PR012884 VH CDR3 | SRVVWYYCSGYGCSHARDYIY |
| | (Chothia) | |
| 82 | PR012884 VH FWR4 | WGQGTQVTVSS |
| | (Chothia) | |
| SEQ ID NO: | PR012885 | anti-CD3 3G11_S10 HCAb hIgG1(C220S, AAA) |
| 159 | PR012885 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSRAHDYAYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 125 | PR012885 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSRAHDYAYWGQGTQVTVSS |
| 10 | PR012885 VH FWR1 | RVHLAESGGGLVQAGGSLRLSCAAS |
| | (Chothia) | |
| 20 | PR012885 VH CDR1 | GSRLNDN |
| | (Chothia) | |
| 31 | PR012885 VH FWR2 | AMAWFRQAPGKEREFVGSI |
| | (Chothia) | |
| 42 | PR012885 VH CDR2 | FWTGGN |
| | (Chothia) | |
| 53 | PR012885 VH FWR3 | TYYADAVKGRLTISRDNAKNALYLQMNSLKPEDTAV |
| | (Chothia) | YYCAA |
| 74 | PR012885 VH CDR3 | SRVVWYYCSGYGCSRAHDYAY |
| | (Chothia) | |
| 82 | PR012885 VH FWR4 | WGQGTQVTVSS |
| | (Chothia) | |
| SEQ ID NO: | PR012886 | anti-CD3 3G11_S11 HCAb hIgG1(C220S, AAA) |
| 160 | PR012886 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSRPRDYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 126 | PR012886 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSRPRDYVYWGQGTQVTVSS |
| 10 | PR012886 VH FWR1 | RVHLAESGGGLVQAGGSLRLSCAAS |
| | (Chothia) | |
| 20 | PR012886 VH CDR1 | GSRLNDN |
| | (Chothia) | |
| 31 | PR012886 VH FWR2 | AMAWFRQAPGKEREFVGSI |
| | (Chothia) | |
| 42 | PR012886 VH CDR2 | FWTGGN |
| | (Chothia) | |
| 53 | PR012886 VH FWR3 | TYYADAVKGRLTISRDNAKNALYLQMNSLKPEDTAV |
| | (Chothia) | YYCAA |
| 75 | PR012886 VH CDR3 | SRVVWYYCSGYGCSRPRDYVY |
| | (Chothia) | |
| 82 | PR012886 VH FWR4 | WGQGTQVTVSS |
| | (Chothia) | |
| SEQ ID NO: | PR012887 | anti-CD3 3G11_S12 HCAb hIgG1(C220S, AAA) |
| 161 | PR012887 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSRSPDYVYWGQGTQVTVSSASEPKSSDKTHTCP |
| | | PCPAPEAAGAPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| 127 | PR012887 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSRSPDYVYWGQGTQVTVSS |
| 10 | PR012887 VH FWR1 | RVHLAESGGGLVQAGGSLRLSCAAS |
| | (Chothia) | |
| 20 | PR012887 VH CDR1 | GSRLNDN |
| | (Chothia) | |
| 31 | PR012887 VH FWR2 | AMAWFRQAPGKEREFVGSI |
| | (Chothia) | |
| 42 | PR012887 VH CDR2 | FWTGGN |
| | (Chothia) | |
| 53 | PR012887 VH FWR3 | TYYADAVKGRLTISRDNAKNALYLQMNSLKPEDTAV |
| | (Chothia) | YYCAA |
| 76 | PR012887 VH CDR3 | SRVVWYYCSGYGCSRSPDYVY |
| | (Chothia) | |
| 82 | PR012887 VH FWR4 | WGQGTQVTVSS |
| | (Chothia) | |
| SEQ ID NO: | PR012888 | anti-CD3 3G11_S13 HCAb hIgG1(C220S, AAA) |
| 162 | PR012888 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSHAANYRYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 128 | PR012888 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSHAANYRYWGQGTQVTVSS |
| 10 | PR012888 VH FWR1 | RVHLAESGGGLVQAGGSLRLSCAAS |
| | (Chothia) | |
| 20 | PR012888 VH CDR1 | GSRLNDN |
| | (Chothia) | |
| 31 | PR012888 VH FWR2 | AMAWFRQAPGKEREFVGSI |
| | (Chothia) | |
| 42 | PR012888 VH CDR2 | FWTGGN |
| | (Chothia) | |
| 53 | PR012888 VH FWR3 | TYYADAVKGRLTISRDNAKNALYLQMNSLKPEDTAV |
| | (Chothia) | YYCAA |
| 77 | PR012888 VH CDR3 | SRVVWYYCSGYGCSHAANYRY |
| | (Chothia) | |
| 82 | PR012888 VH FWR4 | WGQGTQVTVSS |
| | (Chothia) | |
| SEQ ID NO: | PR012889 | anti-CD3 3G11_S14 HCAb hIgG1(C220S, AAA) |
| 163 | PR012889 Heavy Chain | RVHLAESGGGLVQAGGSLRLSCAASGSRLRDNTMAW |
| | Sequence | FRQAPGKEREFVGSIFWTGGNTYYADAVKGRLTISRD |
| | | NAKNALYLQMNSLKPEDTAVYYCAASRVVWYYCSG |
| | | YGCSRAGDYVYWGQGTQVTVSSASEPKSSDKTHTCP |
| | | PCPAPEAAGAPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| 129 | PR012889 VH Sequence | RVHLAESGGGLVQAGGSLRLSCAASGSRLRDNTMAW |
| | | FRQAPGKEREFVGSIFWTGGNTYYADAVKGRLTISRD |
| | | NAKNALYLQMNSLKPEDTAVYYCAASRVVWYYCSG |
| | | YGCSRAGDYVYWGQGTQVTVSS |
| 10 | PR012889 VH FWR1 | RVHLAESGGGLVQAGGSLRLSCAAS |
| | (Chothia) | |
| 22 | PR012889 VH CDR1 | GSRLRDN |
| | (Chothia) | |
| 35 | PR012889 VH FWR2 | TMAWFRQAPGKEREFVGSI |
| | (Chothia) | |
| 42 | PR012889 VH CDR2 | FWTGGN |
| | (Chothia) | |
| 53 | PR012889 VH FWR3 | TYYADAVKGRLTISRDNAKNALYLQMNSLKPEDTAV |
| | (Chothia) | YYCAA |
| 64 | PR012889 VH CDR3 | SRVVWYYCSGYGCSRAGDYVY |
| | (Chothia) | |
| 82 | PR012889 VH FWR4 | WGQGTQVTVSS |
| | (Chothia) | |
| SEQ ID NO: | PR012890 | anti-CD3 3G11_S15 HCAb hIgG1(C220S, AAA) |
| 164 | PR012890 Heavy Chain | RVHLAESGGGLVQAGGSLRLSCAASGSRLNDNAMA |
| | Sequence | WFRQAPGKEREFVGSIFRTGGNAYYADAVKGRLTISR |
| | | DNAKNALYLQMNSLKPEDTAVYYCAASRVVWYYCS |
| | | GYGCSRAGDYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 130 | PR012890 VH Sequence | RVHLAESGGGLVQAGGSLRLSCAASGSRLNDNAMA |
| | | WFRQAPGKEREFVGSIFRTGGNAYYADAVKGRLTISR |
| | | DNAKNALYLQMNSLKPEDTAVYYCAASRVVWYYCS |
| | | GYGCSRAGDYVYWGQGTQVTVSS |
| 10 | PR012890 VH FWR1 | RVHLAESGGGLVQAGGSLRLSCAAS |
| | (Chothia) | |
| 20 | PR012890 VH CDR1 | GSRLNDN |
| | (Chothia) | |
| 31 | PR012890 VH FWR2 | AMAWFRQAPGKEREFVGSI |
| | (Chothia) | |
| 43 | PR012890 VH CDR2 | FRTGGN |
| | (Chothia) | |
| 56 | PR012890 VH FWR3 | AYYADAVKGRLTISRDNAKNALYLQMNSLKPEDTAV |
| | (Chothia) | YYCAA |
| 64 | PR012890 VH CDR3 | SRVVWYYCSGYGCSRAGDYVY |
| | (Chothia) | |
| 82 | PR012890 VH FWR4 | WGQGTQVTVSS |
| | (Chothia) | |
| SEQ ID NO: | PR012891 | anti-CD3 3G11_S16 HCAb hIgG1(C220S, AAA) |
| 165 | PR012891 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCA |
| | | GYGCSRAGNYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 131 | PR012891 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCA |
| | | GYGCSRAGNYVYWGQGTQVTVSS |
| 10 | PR012891 VH FWR1 | RVHLAESGGGLVQAGGSLRLSCAAS |
| | (Chothia) | |
| 20 | PR012891 VH CDR1 | GSRLNDN |
| | (Chothia) | |
| 31 | PR012891 VH FWR2 | AMAWFRQAPGKEREFVGSI |
| | (Chothia) | |
| 42 | PR012891 VH CDR2 | FWTGGN |
| | (Chothia) | |
| 53 | PR012891 VH FWR3 | TYYADAVKGRLTISRDNAKNALYLQMNSLKPEDTAV |
| | (Chothia) | YYCAA |
| 78 | PR012891 VH CDR3 | SRVVWYYCAGYGCSRAGNYVY |
| | (Chothia) | |
| 82 | PR012891 VH FWR4 | WGQGTQVTVSS |
| | (Chothia) | |
| SEQ ID NO: | PR012892 | anti-CD3 3G11_S17 HCAb hIgG1(C220S, AAA) |
| 166 | PR012892 Heavy Chain | RVHLAESGGGLVQAGGSLRLSCAASGSRLNDNAMA |
| | Sequence | WFRQAPGKEREFVGSIFWTGGNTYYADAVKGRLTISR |
| | | DNAKNALYLQMNSLKPEDTAVYYCAASRVVWYYCS |
| | | GYGCSRTGDYVYWGQGTQVTVSSASEPKSSDKTHTC |
| | | PPCPAPEAAGAPSVFLFPPKPKDTLMISRTPEVTCVVV |
| | | DVSHEDPEVKFNWYVDGVEVHNAKTKPREEQYNSTY |
| | | RVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 132 | PR012892 VH Sequence | RVHLAESGGGLVQAGGSLRLSCAASGSRLNDNAMA |
| | | WFRQAPGKEREFVGSIFWTGGNTYYADAVKGRLTISR |
| | | DNAKNALYLQMNSLKPEDTAVYYCAASRVVWYYCS |
| | | GYGCSRTGDYVYWGQGTQVTVSS |
| 10 | PR012892 VH FWR1 | RVHLAESGGGLVQAGGSLRLSCAAS |
| | (Chothia) | |
| 20 | PR012892 VH CDR1 | GSRLNDN |
| | (Chothia) | |
| 31 | PR012892 VH FWR2 | AMAWFRQAPGKEREFVGSI |
| | (Chothia) | |
| 42 | PR012892 VH CDR2 | FWTGGN |
| | (Chothia) | |
| 53 | PR012892 VH FWR3 | TYYADAVKGRLTISRDNAKNALYLQMNSLKPEDTAV |
| | (Chothia) | YYCAA |
| 79 | PR012892 VH CDR3 | SRVVWYYCSGYGCSRTGDYVY |
| | (Chothia) | |
| 82 | PR012892 VH FWR4 | WGQGTQVTVSS |
| | (Chothia) | |

TABLE 18
Polypeptide chain sequences of B7H4xCD3 generated from 3G11 variants.

| | | anti-B7H4xCD3 3G11_S1-Fc-scFv(PR003366), |
|---|---|---|
| SEQ ID NO: | PR012561 | hIgG1(dCH1, KIH, AAA) |
| 185 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSRAADYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S2-Fc-scFv(PR003366), |
| SEQ ID NO: | PR012562 | hIgG1(dCH1, KIH, AAA) |
| 186 | anti-CD3 VH-Fc | RVHLAESGGGLVQAGGSLRLSCAASGSWLYDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSRAGDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S3-Fc-scFv(PR003366), |
| SEQ ID NO: | PR012563 | hIgG1(dCH1, KIH, AAA) |
| 187 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCTRYGCSRPGDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGOGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S4-Fc-scFv(PR003366), |
| SEQ ID NO: | PR012564 | hIgG1(dCH1, KIH, AAA) |
| 188 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSAYGCSGAGNYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S5-Fc-scFv(PR003366), |
| SEQ ID NO: | PR012565 | hIgG1(dCH1, KIH, AAA) |
| 189 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCYRYGCSRGGDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S6-Fc-scFv(PR003366), |
| SEQ ID NO: | PR012566 | hIgG1(dCH1, KIH, AAA) |
| 190 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSRYGCSRSGDYA |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S7-Fc-scFv(PR003366), |
| SEQ ID NO: | PR012567 | hIgG1(dCH1, KIH, AAA) |
| 191 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSRAVDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTESSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S8-Fc-scFv(PR003366), |
| SEQ ID NO: | PR012568 | hIgG1(dCH1, KIH, AAA) |
| 192 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCASYGCSRPVHYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S9-Fc-scFv(PR003366), |
| SEQ ID NO: | PR012569 | hIgG1(dCH1, KIH, AAA) |
| 193 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSHARDYI |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTESSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S10-Fc-scFv(PR003366), |
| SEQ ID NO: | PR012644 | hIgG1(dCH1, KIH, AAA) |
| 194 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSRAHDYA |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S11-Fc-scFv(PR003366), |
| SEQ ID NO: | PR012645 | hIgG1(dCH1, KIH, AAA) |
| 195 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSRPRDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S12-Fc-scFv(PR003366), |
| SEQ ID NO: | PR012646 | hIgG1(dCH1, KIH, AAA) |
| 196 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSRSPDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S13-Fc-scFv(PR003366), |
| SEQ ID NO: | PR012647 | hIgG1(dCH1, KIH, AAA) |
| 197 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSHAANYR |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S14-Fc-scFv(PR003366), |
| SEQ ID NO: | PR012683 | hIgG1(dCH1, KIH, AAA) |
| 198 | anti-CD3 VH-Fc | RVHLAESGGGLVQAGGSLRLSCAASGSRLRDNTMAWFRQ |
| | chain | APGKEREFVGSIFWTGGNTYYADAVKGRLTISRDNAKNAL |
| | | YLQMNSLKPEDTAVYYCAASRVVWYYCSGYGCSRAGDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S15-Fc-scFv(PR003366), |
| SEQ ID NO: | PR012684 | hIgG1(dCH1, KIH, AAA) |
| 199 | anti-CD3 VH-Fc | RVHLAESGGGLVQAGGSLRLSCAASGSRLNDNAMAWFRQ |
| | chain | APGKEREFVGSIFRTGGNAYYADAVKGRLTISRDNAKNALY |
| | | LQMNSLKPEDTAVYYCAASRVVWYYCSGYGCSRAGDYVY |
| | | WGQGTQVTVSSASEPKSSDKTHTCPPCPAPEAAGAPSVFLF |
| | | PPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGOGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S16-Fc-scFv(PR003366), |
| SEQ ID NO: | PR012685 | hIgG1(dCH1, KIH, AAA) |
| 200 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCAGYGCSRAGNY |
| | | VYWGQGTQVTVSSASEPKSSDKTHTCPPCPAPEAAGAPSVF |
| | | LFPPKPKDTLMISRTPEVTCVVVDVSHEDPEVKFNWYVDGV |
| | | EVHNAKTKPREEQYNSTYRVVSVLTVLHQDWLNGKEYKC |
| | | KVSNKALPAPIEKTISKAKGQPREPQVYTLPPCREEMTKNQ |
| | | VSLWCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGOGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| | | anti-B7H4xCD3 3G11_S17-Fc-scFv(PR003366), |
| SEQ ID NO: | PR012686 | hIgG1(dCH1, KiH, AAA) |
| 201 | anti-CD3 VH-Fc | RVHLAESGGGLVQAGGSLRLSCAASGSRLNDNAMAWFRQ |
| | chain | APGKEREFVGSIFWTGGNTYYADAVKGRLTISRDNAKNAL |
| | | YLQMNSLKPEDTAVYYCAASRVVWYYCSGYGCSRTGDYV |
| | | YWGQGTQVTVSSASEPKSSDKTHTCPPCPAPEAAGAPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCREEMTKNQVS |
| | | LWCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 178 | anti-B7H4 scFv-Fc | EIVMTQSPASLSVSPGERATLSCRASQSVSSNLAWYQQKPG |
| | chain | QAPRLLIYGASTRATGIPARVSGGGSGTEFTLTISSLQSEDFA |
| | | VYYCQQYKNWPFTFGPGTKLEIKGGGGSGGGGSGGGGSGG |
| | | GGSQVQLVQSGAEVKKPGSSMRVSCKASEDTFSSYAISWV |
| | | RQAPGQGLEWMGGTAPIFGTTNYAQKFQGRVTITADKSTST |
| | | AYMELSSLRSEDTAVYYCARGGPYFDYWGQGTLVTVSSAS |
| | | EPKSSDKTHTCPPCPAPEAAGAPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVCTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |

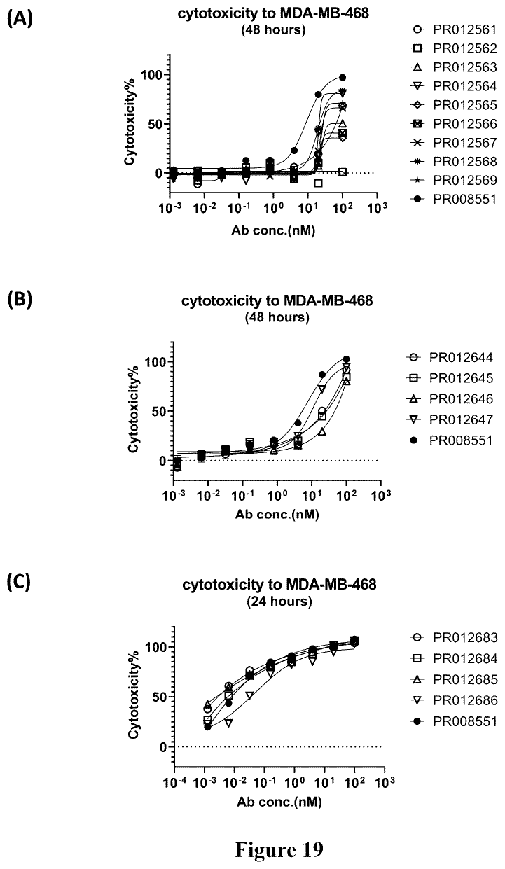

The newly generated B7H4xCD3 bispecific antibodies listed in Table 16 were further evaluated for T cell mediated cytotoxicity by using the RTCA method described in Example 9. In this example, B7H4 highly expressed MDA-MB-468 cells were used as target cells, and human T cells isolated from PBMCs were used as effector cells, the E:T ratio was 10:1. The testing antibodies were 5-fold serial diluted from 100 nM to 8 concentrations. The cells were incubated with testing antibody samples for 24 hours or 48 hours before analysis. The results were shown in FIG. 19, all of B7H4xCD3 bispecific antibodies derived from 3G11 variants retained most of activities to induce cytotoxicity to MDA-MB-468. Some variants (e.g., PR012683, PR012685) even showed the enhanced cytotoxicity potency at low concentrations.

### Example 14: Binding Affinity to CD3 Proteins Determined by BLI Method

To determine the binding affinities of anti-CD3 antibodies to human or cynomolgus monkey CD3e&d heterodimer proteins, bio-layer interferometry (BLI) assays were carried out by using Octet® RED96e.

Biotinylated human CD3e&d protein (Acro Biosystems, CDD-H82W6) or biotinylated cynomolgus CD3e&d protein (Acro Biosystems, CDD-C82W6) was first loaded onto the surface of SA Octet biosensors (Sartorius, 18-5019) to reach capture levels between 0.5-0.8 nm. The loaded biosensors were then dipped in wells containing 2-fold serial dilutions of testing antibodies to detect association signals, followed by dissociation steps in wells containing kinetic buffer. The association phase was 180 seconds, and the dissociation phase was 900 seconds. The sensorgrams were recorded, and the reference signals were subtracted before curve fitting using ForteBio Data Analysis 11.0 software. Association rates (kon) and dissociation rates (koff) were calculated using a simple one-to-one Langmuir binding model. The equilibrium dissociation constant (KD) was calculated as the ratio of koff/kon.

As shown in Table 19, PR010991 (3G11-HCAb) showed strong binding affinity to both human CD3e&d protein and cynomolgus CD3e&d protein, which is consistent to the results by ELISA (FIG. 9). However, PR006361 from prior art can bind only to human CD3e&d protein but not to cynomolgus CD3e&d protein. Besides, PR006364 (A1-HCAb) and its humanized variant PR011629 also showed strong binding affinity to human CD3 protein.

TABLE 19
kinetics parameters of anti-CD3 antibodies binding to CD3 proteins.

| | | Antibody | | | | |
|---|---|---|---|---|---|---|
| Antigen | Antibody | Conc. (nM) | KD (M) | kon(1/Ms) | kdis(1/s) | Full R\hat{ }2 |
| Biotinylated | PR010991 | 1.5-24 | <1.0E−12 | 9.06E+05 | <1.0E−07 | 0.9998 |
| Human | PR006361 | 0.375-12 | 5.75E−11 | 1.71E+06 | 9.84E−05 | 0.9983 |
| CD3 E&D | PR011629 | 0.375-12 | 1.68E−10 | 8.33E+05 | 1.40E−04 | 0.9993 |
| | PR006364 | 0.375-12 | 3.42E−11 | 7.27E+05 | 2.49E−05 | 0.999 |
| Biotinylated | PR010991 | 1.5-24 | <1.0E−12 | 7.08E+05 | <1.0E−07 | 0.998 |
| cynomolgus | PR006361 | 12.5-200 | No binding | N/A | | |
| CD3 E&D | | | | | | |

### Example 15: Generation of ROR1xCD3 Bispecific Antibodies from A1-HCAb

ROR1xCD3 bispecific antibody PR011870 was generated in the format of single-chain VH-in-tandem by composing two ROR1-binding VH domains from previously discovered anti-ROR1 HCAb (PR005340) and one CD3-binding VH domain from A1-HCAb (PR006364) with peptide linkers between the domains. A poly-his tag was appended to the C-terminus of PR011870 to facilitate the purification. The amino acid sequences of ROR1xCD3 PR011870 and anti-ROR1 PR005340 are listed in Table 20.

### PR011870-Protein

PR011870 protein was produced by the method described in Example 4 with affinity-capture using Ni-excel columns (Cytiva).

### PR011870-mRNA

In this example, PR011870-mRNA encoding PR011870 was prepared and encapsulated in Lipid-nanoparticle (LNP).

Briefly, the coding sequence for PR011870 (listed in Table 21) was cloned into a pUC-plasmid vector containing a T7 promoter, human α-globin 5′UTR, mtRNR1 and AES 3′UTR motifs, and a poly-A tail. The plasmid was then linearized and used as transcription template. The mRNA was produced in vitro using T7 RNA polymerase-mediated transcription. N1-Me-Pseudo UTP was used instead of UTP, followed by purification with lithium chloride precipitation.

Lipid-nanoparticle (LNP) formulations were prepared by mixing ethanol and aqueous phase at a 1:3 volumetric ratio in a microfluidic device. The ethanol phase was prepared by solubilizing a mixture of ionizable lipid, 1, 2-distearoyl-sn-glycero-3-phosphocholine (DSPC), cholesterol and PEG-lipid at a molar ratio of 50:10:38.5:1.5. The aqueous phase was prepared in citrate buffer with PR011870-mRNA. LNPs were dialyzed against PBS, and concentrated to desired concentrations. Sucrose was added as a cryoprotectant. The formulated LNP was stored at −70° C. until further use. All formulations were tested for particle size, distribution, RNA concentration and encapsulation.

### Fluc-mRNA

The firefly luciferase (Fluc) encoding mRNA was also prepared in the same procedure (sequence in Table 21), and then encapsulated in LNP. Fluc-mRNA is serving as negative control in other examples.

TABLE 20
Sequences of ROR1xCD3 PR011870 and anti-ROR1 PR005340.

| | | anti-ROR1xCD3 PR005340_VH-(G4S)3- |
|---|---|---|
| SEQ ID NO: | PR011870 | PR005340_VH-(G4S)3-PR006364_VH-6His |
| 184 | Polypeptide Sequence | EVQLVESGGGLVKPGGSLRLSCAASGFTFSDFYMSWIR |
| | | QAPGKGLEWISYISSSGSTRYYADSVKGRFTISRDNAKN |
| | | SLYLQMNSLRAEDTAIYYCARDIPSSSSDWVDLQFDYW |
| | | GQGTLVTVSSGGGGSGGGGSGGGGSEVQLVESGGGLV |
| | | KPGGSLRLSCAASGFTFSDFYMSWIRQAPGKGLEWISYI |
| | | SSSGSTRYYADSVKGRFTISRDNAKNSLYLQMNSLRAE |
| | | DTAIYYCARDIPSSSSDWVDLQFDYWGQGTLVTVSSGG |
| | | GGSGGGGSGGGGSRIRLVESGGGLVQAGGSLRLSCAAS |
| | | GRTSGIPAMAWFRQAPGKEREFVAMAWSGGGTIYGDS |
| | | VKNRFTISRDNTQNTIFLQMNSLKPEDTALYYCAAGRS |
| | | TSNRADQYNYWGQGTQVTVSSASHHHHHH |
| SEQ ID NO: | PR005340 | anti-ROR1 R1015P135A09 HCAb hIgG1(C220S) |
| 139 | PR005340 | EVQLVESGGGLVKPGGSLRLSCAASGFTFSDYYMSWIR |
| | Heavy Chain | QAPGKGLEWISYISSSGSTIHYADSVKGRFTISRDNAKN |
| | Sequence | SLYLQMNSLRAEDTAIYYCARDIPSSSSDWVSLQFDYW |
| | | GQGTLVTVSSEPKSSDKTHTCPPCPAPELLGGPSVFLFPP |
| | | KPKDTLMISRTPEVTCVVVDVSHEDPEVKFNWYVDGV |
| | | EVHNAKTKPREEQYNSTYRVVSVLTVLHQDWLNGKE |
| | | YKCKVSNKALPAPIEKTISKAKGQPREPQVYTLPPSREE |
| | | MTKNQVSLTCLVKGFYPSDIAVEWESNGQPENNYKTT |
| | | PPVLDSDGSFFLYSKLTVDKSRWQQGNVFSCSVMHEA |
| | | LHNHYTQKSLSLSPGK |
| 105 | PR005340 VH | EVQLVESGGGLVKPGGSLRLSCAASGFTFSDYYMSWIR |
| | Sequence | QAPGKGLEWISYISSSGSTIHYADSVKGRFTISRDNAKN |
| | | SLYLQMNSLRAEDTAIYYCARDIPSSSSDWVSLQFDYW |
| | | GQGTLVTVSS |
| 5 | PR005340 VH FWR1 | EVQLVESGGGLVKPGGSLRLSCAAS |
| | (Chothia) | |
| 15 | PR005340 VH CDR1 | GFTFSDY |
| | (Chothia) | |
| 27 | PR005340 VH FWR2 | YMSWIRQAPGKGLEWISYI |
| | (Chothia) | |
| 39 | PR005340 VH CDR2 | SSSGST |
| | (Chothia) | |
| 48 | PR005340 VH FWR3 | IHYADSVKGRFTISRDNAKNSLYLQMNSLRAEDTAIYY |
| | (Chothia) | CAR |
| 60 | PR005340 VH CDR3 | DIPSSSSDWVSLQFDY |
| | (Chothia) | |
| 81 | PR005340 VH FWR4 | WGQGTLVTVSS |
| | (Chothia) | |

TABLE 21
Coding sequences of PR011870 and Fluc.

| | Sequence | SEQ ID NO |
|---|---|---|
| PR011870 | ATGGAGACCGACACCCTGCTGCTGT | 204 |
| Coding | GGGTGCTGCTGCTGTGGGTGCCCGG | |
| sequence | ATCCACCGGCGAGGTGCAGCTGGTG | |
| | GAGTCTGGGGGAGGCTTGGTCAAGC | |
| | CTGGAGGATCCCTGAGACTCTCCTG | |
| | TGCAGCCTCTGGATTCACCTTCAGT | |
| | GACTTCTACATGAGTTGGATCCGCC | |
| | AGGCTCCAGGGAAGGGGCTGGAGTG | |
| | GATTTCATACATTAGTAGTAGTGGT | |
| | AGCACCCGCTACTACGCAGACTCTG | |
| | TGAAGGGCCGATTCACCATCTCCAG | |
| | GGACAACGCCAAGAACTCACTGTAT | |
| | CTGCAAATGAACAGCCTGAGAGCCG | |
| | AGGACACGGCCATATATTATTGTGC | |
| | GAGAGATATCCCATCGTCTAGCAGC | |
| | GACTGGGTTGATCTACAGTTTGACT | |
| | ACTGGGGCCAGGGAACCCTGGTCAC | |
| | CGTCTCCTCAGGCGGAGGAGGTTCG | |
| | GGAGGCGGTGGTAGTGGTGGAGGAG | |
| | GCTCTGAGGTGCAGCTGGTGGAGTC | |
| | TGGGGGAGGCTTGGTCAAGCCTGGA | |
| | GGATCCCTGAGACTCTCCTGTGCAG | |
| | CCTCTGGATTCACCTTCAGTGACTT | |
| | CTACATGAGTTGGATCCGCCAGGCT | |
| | CCAGGGAAGGGGCTGGAGTGGATTT | |
| | CATACATTAGTAGTAGTGGTAGCAC | |
| | CCGCTACTACGCAGACTCTGTGAAG | |
| | GGCCGATTCACCATCTCCAGGGACA | |
| | ACGCCAAGAACTCACTGTATCTGCA | |
| | AATGAACAGCCTGAGAGCCGAGGAC | |
| | ACGGCCATATATTATTGTGCGAGAG | |
| | ATATCCCATCGTCTAGCAGCGACTG | |
| | GGTTGATCTACAGTTTGACTACTGG | |
| | GGCCAGGGAACCCTGGTCACCGTCT | |
| | CCTCAGGAGGCGGTGGTAGTGGTGG | |
| | AGGAGGCTCTGGTGGCGGAGGGTCT | |
| | CGAATACGTCTGGTAGAGTCTGGGG | |
| | GAGGATTGGTGCAGGCTGGGGGCTC | |
| | TCTGAGACTCTCCTGTGCAGCCTCT | |
| | GGACGCACCTCCGGTATTCCTGCCA | |
| | TGGCCTGGTTCCGCCAGGCTCCAGG | |
| | GAAGGAGCGTGAGTTTGTAGCTATG | |
| | GCATGGAGTGGTGGTGGCACAATCT | |
| | ATGGAGACTCCGTGAAAAACCGATT | |
| | CACCATCTCCAGAGACAACACCCAG | |
| | AACACGATATTTCTGCAAATGAACA | |
| | GCCTGAAACCTGAGGACACGGCCCT | |
| | TTATTACTGTGCAGCAGGTCGGAGC | |
| | ACTAGTAATAGGGCCGACCAGTATA | |
| | ACTACTGGGGCCAGGGGACCCAGGT | |
| | CACCGTCTCCTCAGCTAGCCATCAT | |
| | CACCATCACCATTGA | |
| Fluc | ATGGAGGACGCCAAGAACATCAAGA | 205 |
| Coding | AGGGCCCCGCCCCCTTCTACCCCCT | |
| sequence | GGAGGACGGCACCGCCGGCGAGCAG | |
| | CTGCACAAGGCCATGAAGCGGTACG | |
| | CCCTGGTGCCCGGCACCATCGCCTT | |
| | CACCGACGCCCACATCGAGGTGGAC | |
| | ATCACCTACGCCGAGTACTTCGAGA | |
| | TGAGCGTGCGGCTGGCCGAGGCCAT | |
| | GAAGCGGTACGGCCTGAACACCAAC | |
| | CACCGGATCGTGGTGTGCAGCGAGA | |
| | ACAGCCTGCAGTTCTTCATGCCCGT | |
| | GCTGGGCGCCCTGTTCATCGGCGTG | |
| | GCCGTGGCCCCCGCCAACGACATCT | |
| | ACAACGAGCGGGAGCTGCTGAACAG | |
| | CATGGGCATCAGCCAGCCCACCGTG | |
| | GTGTTCGTGAGCAAGAAGGGCCTGC | |
| | AGAAGATCCTGAACGTGCAGAAGAA | |
| | GCTGCCCATCATCCAGAAGATCATC | |
| | ATCATGGACAGCAAGACCGACTACC | |
| | AGGGCTTCCAGAGCATGTACACCTT | |
| | CGTGACCAGCCACCTGCCCCCCGGC | |
| | TTCAACGAGTACGACTTCGTGCCCG | |
| | AGAGCTTCGACCGGGACAAGACCAT | |
| | CGCCCTGATCATGAACAGCAGCGGC | |
| | AGCACCGGCCTGCCCAAGGGCGTGG | |
| | CCCTGCCCCACCGGACCGCCTGCGT | |
| | GCGGTTCAGCCACGCCCGGGACCCC | |
| | ATCTTCGGCAACCAGATCATCCCCG | |
| | ACACCGCCATCCTGAGCGTGGTGCC | |
| | CTTCCACCACGGCTTCGGCATGTTC | |
| | ACCACCCTGGGCTACCTGATCTGCG | |
| | GCTTCCGGGTGGTGCTGATGTACCG | |
| | GTTCGAGGAGGAGCTGTTCCTGCGG | |
| | AGCCTGCAGGACTACAAGATCCAGA | |
| | GCGCCCTGCTGGTGCCCACCCTGTT | |
| | CAGCTTCTTCGCCAAGAGCACCCTG | |
| | ATCGACAAGTACGACCTGAGCAACC | |
| | TGCACGAGATCGCCAGCGGCGGCGC | |
| | CCCCCTGAGCAAGGAGGTGGGCGAG | |
| | GCCGTGGCCAAGCGGTTCCACCTGC | |
| | CCGGCATCCGGCAGGGCTACGGCCT | |
| | GACCGAGACCACCAGCGCCATCCTG | |
| | ATCACCCCCGAGGGCGACGACAAGC | |
| | CCGGCGCCGTGGGCAAGGTGGTGCC | |
| | CTTCTTCGAGGCCAAGGTGGTGGAC | |
| | CTGGACACCGGCAAGACCCTGGGCG | |
| | TGAACCAGCGGGGCGAGCTGTGCGT | |
| | GCGGGGCCCCATGATCATGAGCGGC | |
| | TACGTGAACAACCCCGAGGCCACCA | |
| | ACGCCCTGATCGACAAGGACGGCTG | |
| | GCTGCACAGCGGCGACATCGCCTAC | |
| | TGGGACGAGGACGAGCACTTCTTCA | |
| | TCGTGGACCGGCTGAAGAGCCTGAT | |
| | CAAGTACAAGGGCTACCAGGTGGCC | |
| | CCCGCCGAGCTGGAGAGCATCCTGC | |
| | TGCAGCACCCCAACATCTTCGACGC | |
| | CGGCGTGGCCGGCCTGCCCGACGAC | |
| | GACGCCGGCGAGCTGCCCGCCGCCG | |
| | TGGTGGTGCTGGAGCACGGCAAGAC | |
| | CATGACCGAGAAGGAGATCGTGGAC | |
| | TACGTGGCCAGCCAGGTGACCACCG | |
| | CCAAGAAGCTGCGGGGCGGCGTGGT | |
| | GTTCGTGGACGAGGTGCCCAAGGGC | |
| | CTGACCGGCAAGCTGGACGCCCGGA | |
| | AGATCCGGGAGATCCTGATCAAGGC | |
| | CAAGAAGGGCGGCAAGATCGCCGTG | |
| | TGA | |

### Example 16: T Cell-Mediated Cytotoxicity of ROR1xCD3 Bispecific Antibodies

The T cell mediated cytotoxicity of ROR1xCD3 bispecific antibodies including PR011870-protein and PR011870-mRNA to specific target cells were determined by RTCA method as described in Example 9. In this example, ROR1 highly expressed tumor cell PANC-1 (ATCC, CRL-1469) were used as target cells, and isolated human T cells were used as effector cells, the E:T ratio was 5:1. The cells were incubated with testing samples (PR011870-protein, PR011870-mRNA and Fluc-mRNA prepared in Example 15) for 27 hours before analysis.

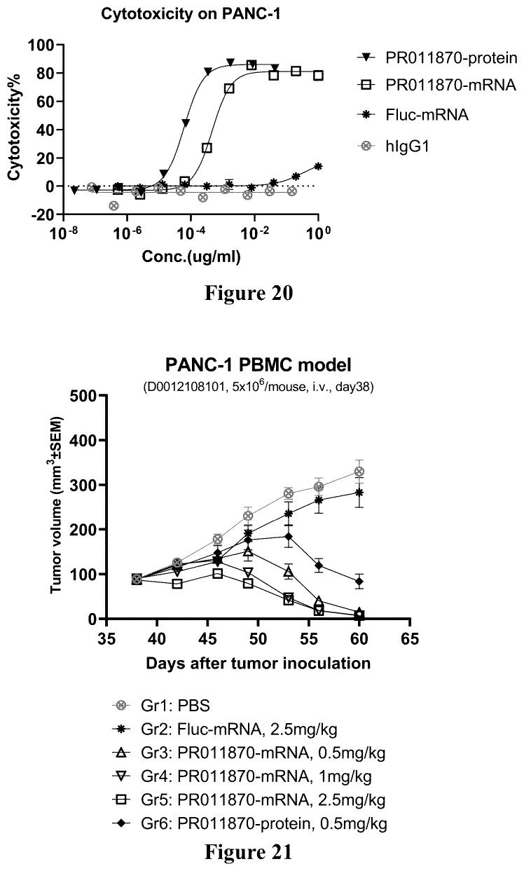

The results are shown in FIG. 20 and Table 22. ROR1xCD3 bispecific antibody PR0011870 in both recombinant protein form and mRNA form can raise strong and dose-dependent cytotoxicity to the target cell PANC-1, suggesting that A1-HCAb can be used to generate multiple TAAxCD3 engager bispecific antibodies no matter to targeting B7H4 or ROR1 or other tumor associated antigens.

TABLE 22
Characteristic parameters of cytotoxicity to PANC-1.

| Testing sample | EC50 (pg/ml) |
|---|---|
| PR011870-protein | 0.061 |
| PR011870-mRNA | 0.46 |

### Example 17: In Vivo Pharmacology Study for ROR1xCD3 Bispecific Antibody

In vivo pharmacology study was conducted by using NCG mice (GemPharmatech, Strain NO. T001475) to establish PANC-1 tumor model and human PBMC immune system. Briefly, on the day of cell inoculation, each NCG mouse was subcutaneously inoculated with PANC-1 (ATCC, CRL-1469) cells. When the mean tumor volume of each group of mice reached 88 mm³, the mice were divided into 6 groups, and three administrations for each testing sample were performed once per week (at day 39, day 46 and day 53 after the day of tumor cell inoculation) through tail veins, as listed in Table 23. After the start of administration, the body weight and the tumor volume were measured twice a week. The tumor volume was calculated as follows: tumor volume (mm³)=0.5×long diameter of tumor×short diameter of tumor². The data were analyzed using t-test.

The result of tumor volume changes is shown in FIG. 21, and the tumor growth inhibition rate TGI (%) at day 60 is summarized in Table 23. Specifically, the mean tumor volume of the PBS vehicle group of mice at day 60 after inoculation was 330 mm³. The mean tumor volume of Fluc-mRNA (2.5 mg/kg) treatment group at day 60 after inoculation was 283 mm³, with TGI (%) of 14.18%. The mean tumor volume of PR011870-mRNA (2.5 mg/kg) treatment group at day 60 was 7 mm³ with TGI (%) of 97.92% which showed significant difference (p value<0.05) compared to the vehicle group. PR011870-mRNA showed potent anti-tumor activity at 1.0 mg/kg and 0.5 mg/kg with TGI (%) of 97.73% and 95.53% respectively (p value<0.05). The mean tumor volume of PR011870-protein treatment group was 83 mm³ with TGI (%) of 74.72% (p value<0.05). During the treatment, all animals showed good tolerance for the treatments, with no obvious weight loss.

This study demonstrated that ROR1xCD3 bispecific antibody PR011870 in both recombinant protein form and mRNA form are very efficient and potent in treating PANC-1 tumors. Surprisingly, mRNA form even showed stronger anti-tumor activity that protein form of same dose, bringing almost complete response, i.e. TGI=95.53% vs. TGI=74.72%, and the efficacy of mRNA form could be more persistent.

TABLE 23
Summary of in vivo pharmacology study.

| Group No. | Testing sample | Dose | TGI (%) at day 60 |
|---|---|---|---|
| Gr1 | PBS buffer | | N/A |
| Gr2 | Fluc-mRNA | 2.5 mg/kg | 14.18% |
| Gr3 | PR011870-mRNA | 0.5 mg/kg | 95.53% |
| Gr4 | PR011870-mRNA | 1.0 mg/kg | 97.73% |
| Gr5 | PR011870-mRNA | 2.5 mg/kg | 97.92% |
| Gr6 | PR011870-protein | 0.5 mg/kg | 74.72% |

This study also approves the bioactivity of anti-CD3 antibodies of this invention, which can bring significant values of generating innovative biologics to treat diseases.

It is to be understood that the foregoing description of two preferred embodiments is intended to be purely illustrative of the principles of the disclosure, rather than exhaustive thereof, and that changes and variations will be apparent to those skilled in the art, and that the present disclosure is not intended to be limited other than expressly set forth in the following claims.

1. (canceled)

2. A CD3-targeting antibody comprising a heavy chain variable region comprising HCDR1, HCDR2 and HCDR3, wherein,
the HCDR1, HCDR2 and HCDR3 comprise the amino acid sequence set forth in SEQ ID NOs: 17, 41 and 62, respectively; or
the HCDR1, HCDR2 and HCDR3 comprise the amino acid sequence set forth in SEQ ID NOs: 18, 41 and 63, respectively; or
the HCDR1, HCDR2 and HCDR3 comprise the amino acid sequence set forth in SEQ ID NOs: 20, 42 and 64, respectively; or
the HCDR1, HCDR2 and HCDR3 comprise the amino acid sequence set forth in SEQ ID NOs: 17, 41 and 65, respectively; or
the HCDR1, HCDR2 and HCDR3 comprise the amino acid sequence set forth in SEQ ID NOs: 20, 42 and 66, respectively; or
the HCDR1, HCDR2 and HCDR3 comprise the amino acid sequence set forth in SEQ ID NOs: 21, 42 and 64, respectively; or
the HCDR1, HCDR2 and HCDR3 comprise the amino acid sequence set forth in SEQ ID NOs: 20, 42 and 67, respectively; or
the HCDR1, HCDR2 and HCDR3 comprise the amino acid sequence set forth in SEQ ID NOs: 20, 42 and 68, respectively; or
the HCDR1, HCDR2 and HCDR3 comprise the amino acid sequence set forth in SEQ ID NOs: 20, 42 and 69, respectively; or
the HCDR1, HCDR2 and HCDR3 comprise the amino acid sequence set forth in SEQ ID NOs: 20, 42 and 70, respectively; or
the HCDR1, HCDR2 and HCDR3 comprise the amino acid sequence set forth in SEQ ID NOs: 20, 42 and 71, respectively; or
the HCDR1, HCDR2 and HCDR3 comprise the amino acid sequence set forth in SEQ ID NOs: 20, 42 and 72, respectively; or
the HCDR1, HCDR2 and HCDR3 comprise the amino acid sequence set forth in SEQ ID NOs: 20, 42 and 73, respectively; or
the HCDR1, HCDR2 and HCDR3 comprise the amino acid sequence set forth in SEQ ID NOs: 20, 42 and 74, respectively; or
the HCDR1, HCDR2 and HCDR3 comprise the amino acid sequence set forth in SEQ ID NOs: 20, 42 and 75, respectively; or
the HCDR1, HCDR2 and HCDR3 comprise the amino acid sequence set forth in SEQ ID NOs: 20, 42 and 76, respectively; or
the HCDR1, HCDR2 and HCDR3 comprise the amino acid sequence set forth in SEQ ID NOs: 20, 42 and 77, respectively; or
the HCDR1, HCDR2 and HCDR3 comprise the amino acid sequence set forth in SEQ ID NOs: 22, 42 and 64, respectively; or
the HCDR1, HCDR2 and HCDR3 comprise the amino acid sequence set forth in SEQ ID NOs: 20, 43 and 64, respectively; or
the HCDR1, HCDR2 and HCDR3 comprise the amino acid sequence set forth in SEQ ID NOs: 20, 42 and 78, respectively; or
the HCDR1, HCDR2 and HCDR3 comprise the amino acid sequence set forth in SEQ ID NOs: 20, 42 and 79, respectively; the CDR regions are defined as Chothia numbering system.

3. The antibody according to claim 2, wherein the heavy chain variable region (VH) comprises the amino acid sequence as set forth in any one of SEQ ID NOs: 107-108 and SEQ ID NOs: 110-132;
or, the VH fragment has at least 90%, at least 95%, at least 98%, at least 99% of sequence identity to the amino acid sequence as set forth in any one of SEQ ID NOs: 107-108 and SEQ ID NOs: 110-132.

4. The antibody according to claim 2, wherein the antibody further comprises a heavy chain constant region; preferably, the heavy chain constant region is derived from hIgG1, hIgG2, hIgG3 and hIgG4 and a variant thereof; more preferably, the heavy chain constant region is derived from hIgG1.

5. The antibody according to claim 4, wherein the antibody further comprises one or more mutations at heavy chain constant region; preferably, the mutation could be selected from L234A, L235A, G237A, P329G (Eu numbering) or any combination.

6. The antibody according to claim 3, wherein the antibody is a heavy chain only antibody comprising an amino acid sequence as set forth in any one of SEQ ID NOs: 141-142 and SEQ ID NOs: 144-166.

7. A bispecific antibody comprising a first antigen-binding fragment targeting CD3 and a second antigen-binding fragment targeting tumor associated antigen (TAA); wherein the first antigen-binding fragment targeting CD3 is as described in claim 2; preferably, the TAA could be any one of CD19, BCMA, TSHR, CD171, CS-1, CLL-1, GD3, Tn Ag, FLT3, CD38, CD123, CD44v6, B7H3, B7H4, KIT, IL-13Ra2, IL-11Ra, PSCA, PSMA, PRSS21, VEGFR2, LewisY, CD24, PDGFR-beta, SSEA-4, MUC1, EGFR, NCAM, CAIX, LMP2, EphA2, sLe, GM3, TGS5, HMWMAA, GD2, FOLR1, FOLR2, TEM1/CD248, TEM7R, CLDN6, CLDN18.2, GPRC5D, CXORF61, CD97, CD179a, ALK, PLAC1, GloboH, NY-BR-1, UPK2, HAVCR1, ADRB3, PANX3, GPR20, LY6K, OR51E2, TAARP, WT1, ETV6-AML, SPA17, XAGE1, Tie 2, MAD-CT-1, MAD-CT-2, FOSL1, hTERT, ML-IAP, ERG, NA17, PAX3, AR, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLECi2A, BST2, EMR2, LY75, GPC3, FCRL5, IGLL1, CD20, CD30, HER2, ROR1, FLT3, TAAG72, CD22, CD33, GD2, gp100Tn, FAP, TYR, EPCAM, CEA, IGF-1R, EphB2, MSLN, CDH17, CD32b, EGFRvIII, GPNMB, GPR64, HER3, LRP6, LYPD8, NKG2D, SLC34A2, SLC39A6, SLITRK6, GUCY2C and TACSTD2; more preferably, BCMA, B7H4 or ROR1.

8. The bispecific antibody according to claim 7, wherein the second antigen-binding fragment targets B7H4; preferably, the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; the CDR regions are defined as Chothia numbering system.

9. The bispecific antibody according to claim 7, wherein the second antigen-binding fragment targets BCMA; preferably,
the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 12, 36 and 57, respectively; the CDR regions are defined as Chothia numbering system.

10. The bispecific antibody according to claim 7, wherein the second antigen-binding fragment targets ROR1; preferably,
the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 15, 39 and 60, respectively; the CDR regions are defined as Chothia numbering system.

11. The bispecific antibody according to claim 7, wherein,
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 62, respectively; the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 12, 36 and 57, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 18, 41 and 63, respectively; the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 12, 36 and 57, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 62, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 18, 41 and 63, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 64, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 65, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 66, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 21, 42 and 64, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 67, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 68, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 69, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 70, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 71, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 72, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 73, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 74, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 75, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 76, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 77, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 22, 42 and 64, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 43 and 64, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 78, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 20, 42 and 79, respectively; the second antigen-binding fragment comprises one light chain variable region (VL) and one heavy chain variable region (VH), the VL region comprises an LCDR1, an LCDR2 and an LCDR3, wherein the LCDR1, LCDR2 and LCDR3 comprise the amino acid sequence set forth in SEQ ID NOs: 88, 93 and 98, respectively, the VH region comprises an HCDR1, an HCDR2 and an HCDR3, wherein the HCDR1, HCDR2 and HCDR3 comprise the amino acid sequence set forth in SEQ ID NOs: 14, 38 and 59, respectively; or
the first antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 17, 41 and 62, respectively; the second antigen-binding fragment comprises one heavy chain variable region (VH), the VH region comprises an HCDR1, an HCDR2 and an HCDR3, wherein the HCDR1, HCDR2 and HCDR3 comprise the amino acid sequence set forth in SEQ ID NOs: 15, 39 and 60, respectively; the CDR regions are defined as Chothia numbering system.

12. The bispecific antibody according to claim 7, wherein,
the first antigen-binding fragment comprises one heavy chain variable region (VH), the VH region comprises the amino acid sequence set forth in SEQ ID NO: 107, the second antigen-binding fragment comprises one heavy chain variable region (VH), the VH region comprises the amino acid sequence set forth in SEQ ID NO: 101; or,
the first antigen-binding fragment comprises one heavy chain variable region (VH), the VH region comprises the amino acid sequence set forth in SEQ ID NO: 108, the second antigen-binding fragment comprises one heavy chain variable region (VH), the VH region comprises the amino acid sequence set forth in SEQ ID NO: 101; or,
the first antigen-binding fragment comprises one heavy chain variable region (VH), the VH region comprises the amino acid sequence set forth in SEQ ID NO: 107, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 108, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 110, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 111, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 112, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 113, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 114, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 115, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 116, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 117, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 118, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 119, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 120, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 121, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 122, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 123, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 124, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 125, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 126, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 127, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 128, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 129, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 130, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 131, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 132, the second antigen-binding fragment comprises one light chain variable region (VL) and one heavy chain variable region (VH), the VL region comprises the amino acid sequence set forth in SEQ ID NO: 134, the VH region comprises the amino acid sequence set forth in SEQ ID NO: 103; or,
the first antigen-binding fragment comprises one heavy chain variable region (VH), the VH region comprises the amino acid sequence set forth in SEQ ID NO: 107, the second antigen-binding fragment comprises one heavy chain variable region (VH), the VH region comprises the amino acid sequence set forth in SEQ ID NO: 105.

13. The bispecific antibody according to claim 7, comprising one polypeptide with the amino acid sequence set forth in SEQ ID NO: 172; or comprising one polypeptide with the amino acid sequence set forth in SEQ ID NO: 173; or comprising two polypeptides, wherein, the first polypeptide comprises the amino acid sequence set forth in SEQ ID NO: 175, the second polypeptide comprises the amino acid sequence set forth in SEQ ID NO: 170; or
comprising two polypeptides, wherein, the first polypeptide comprises the amino acid sequence set forth in SEQ ID NO: 176, the second polypeptide comprises the amino acid sequence set forth in SEQ ID NO: 170; or
comprising two polypeptides, wherein, the first polypeptide comprises the amino acid sequence set forth in SEQ ID NO: 177, the second polypeptide comprises the amino acid sequence set forth in SEQ ID NO: 178; or
comprising two polypeptides, wherein, the first polypeptide comprises the amino acid sequence set forth in SEQ ID NO: 179, the second polypeptide comprises the amino acid sequence set forth in SEQ ID NO: 178; or
comprising two polypeptides, wherein, the first polypeptide comprises the amino acid sequence set forth in SEQ ID NO: 180, the second polypeptide comprises the amino acid sequence set forth in SEQ ID NO: 178; or
comprising two polypeptides, wherein, the first polypeptide comprises the amino acid sequence set forth in SEQ ID NO: 181, the second polypeptide comprises the amino acid sequence set forth in SEQ ID NO: 178; or
comprising two polypeptides, wherein, the first polypeptide comprises the amino acid sequence set forth in SEQ ID NO: 182, the second polypeptide comprises the amino acid sequence set forth in SEQ ID NO: 178; or
comprising two polypeptides, wherein, the first polypeptide comprises the amino acid sequence set forth in SEQ ID NO: 183, the second polypeptide comprises the amino acid sequence set forth in SEQ ID NO: 178; or
comprising two polypeptides, wherein, the first polypeptide comprises the amino acid sequence set forth in SEQ ID NO: 185, the second polypeptide comprises the amino acid sequence set forth in SEQ ID NO: 178; or
comprising two polypeptides, wherein, the first polypeptide comprises the amino acid sequence set forth in SEQ ID NO: 186, the second polypeptide comprises the amino acid sequence set forth in SEQ ID NO: 178; or
comprising two polypeptides, wherein, the first polypeptide comprises the amino acid sequence set forth in SEQ ID NO: 187, the second polypeptide comprises the amino acid sequence set forth in SEQ ID NO: 178; or
comprising two polypeptides, wherein, the first polypeptide comprises the amino acid sequence set forth in SEQ ID NO: 188, the second polypeptide comprises the amino acid sequence set forth in SEQ ID NO: 178; or
comprising two polypeptides, wherein, the first polypeptide comprises the amino acid sequence set forth in SEQ ID NO: 189, the second polypeptide comprises the amino acid sequence set forth in SEQ ID NO: 178; or
comprising two polypeptides, wherein, the first polypeptide comprises the amino acid sequence set forth in SEQ ID NO: 190, the second polypeptide comprises the amino acid sequence set forth in SEQ ID NO: 178; or
comprising two polypeptides, wherein, the first polypeptide comprises the amino acid sequence set forth in SEQ ID NO: 191, the second polypeptide comprises the amino acid sequence set forth in SEQ ID NO: 178; or
comprising two polypeptides, wherein, the first polypeptide comprises the amino acid sequence set forth in SEQ ID NO: 192, the second polypeptide comprises the amino acid sequence set forth in SEQ ID NO: 178; or
comprising two polypeptides, wherein, the first polypeptide comprises the amino acid sequence set forth in SEQ ID NO: 193, the second polypeptide comprises the amino acid sequence set forth in SEQ ID NO: 178; or
comprising two polypeptides, wherein, the first polypeptide comprises the amino acid sequence set forth in SEQ ID NO: 194, the second polypeptide comprises the amino acid sequence set forth in SEQ ID NO: 178; or
comprising two polypeptides, wherein, the first polypeptide comprises the amino acid sequence set forth in SEQ ID NO: 195, the second polypeptide comprises the amino acid sequence set forth in SEQ ID NO: 178; or
comprising two polypeptides, wherein, the first polypeptide comprises the amino acid sequence set forth in SEQ ID NO: 196, the second polypeptide comprises the amino acid sequence set forth in SEQ ID NO: 178; or
comprising two polypeptides, wherein, the first polypeptide comprises the amino acid sequence set forth in SEQ ID NO: 197, the second polypeptide comprises the amino acid sequence set forth in SEQ ID NO: 178; or
comprising two polypeptides, wherein, the first polypeptide comprises the amino acid sequence set forth in SEQ ID NO: 198, the second polypeptide comprises the amino acid sequence set forth in SEQ ID NO: 178; or
comprising two polypeptides, wherein, the first polypeptide comprises the amino acid sequence set forth in SEQ ID NO: 199, the second polypeptide comprises the amino acid sequence set forth in SEQ ID NO: 178; or
comprising two polypeptides, wherein, the first polypeptide comprises the amino acid sequence set forth in SEQ ID NO: 200, the second polypeptide comprises the amino acid sequence set forth in SEQ ID NO: 178; or
comprising two polypeptides, wherein, the first polypeptide comprises the amino acid sequence set forth in SEQ ID NO: 201, the second polypeptide comprises the amino acid sequence set forth in SEQ ID NO: 178; or
comprising one polypeptide with the amino acid sequence set forth in SEQ ID NO: 184.

14. A polynucleotide encoding the CD3-targeting antibody of claim 2 or a bispecific antibody; wherein the bispecific antibody comprises a first antigen-binding fragment targeting CD3 and a second antigen-binding fragment targeting tumor associated antigen (TAA); wherein the first antigen-binding fragment targeting CD3 is the CD3-targeting antibody;
preferably, the polynucleotide is selected from DNA or RNA; more preferably, the polynucleotide is mRNA.

15. A vector comprising the polynucleotide of claim 14.

16. A cell comprising the CD3-targeting antibody of claim 2, or a bispecific antibody, a polynucleotide encoding the CD3-targeting antibody or the bispecific antibody, or a vector comprising the polynucleotide;
wherein the bispecific antibody comprises a first antigen-binding fragment targeting CD3 and a second antigen-binding fragment targeting tumor associated antigen (TAA); wherein the first antigen-binding fragment targeting CD3 is the CD3-targeting antibody.

17. A method for preparing the CD3-targeting antibody of claim 2 or a bispecific antibody, comprising a step of cultivating a cell comprising a polynucleotide encoding the CD3-targeting antibody or the bispecific antibody or a vector comprising the polynucleotide to obtain the CD3-targeting antibody or the bispecific antibody from culture;
wherein the bispecific antibody comprises a first antigen-binding fragment targeting CD3 and a second antigen-binding fragment targeting tumor associated antigen (TAA); wherein the first antigen-binding fragment targeting CD3 is the CD3-targeting antibody.

18. A pharmaceutical composition comprising the CD3-targeting antibody of claim 2 or a bispecific antibody or a polynucleotide encoding the CD3-targeting antibody or the bispecific antibody, or a vector comprising the polynucleotide, or a cell comprising the CD3-targeting antibody, the bispecific antibody, the polynucleotide or the vector, and optionally a pharmaceutically acceptable carrier;
wherein the bispecific antibody comprises a first antigen-binding fragment targeting CD3 and a second antigen-binding fragment targeting tumor associated antigen (TAA); wherein the first antigen-binding fragment targeting CD3 is the CD3-targeting antibody.

19. (canceled)

20. (canceled)

21. A method for preventing and/or treating diseases in a subject in need thereof, comprising administering an effective amount of the CD3-targeting antibody of claim 2, a bispecific antibody or a polynucleotide encoding the CD3-targeting antibody or the bispecific antibody, or a vector comprising the polynucleotide, or a cell comprising the CD3-targeting antibody, the bispecific antibody, the polynucleotide or the vector, or a pharmaceutical composition comprising the CD3-targeting antibody, the bispecific antibody, the polynucleotide, the vector or the cell;
wherein the bispecific antibody comprises a first antigen-binding fragment targeting CD3 and a second antigen-binding fragment targeting tumor associated antigen (TAA); wherein the first antigen-binding

22. The method of claim 21, wherein the disease is selected from the group consisting of myeloma, prostate cancer, liver cancer, kidney cancer, pancreatic cancer, breast cancer, cervical cancer, ovarian cancer, colorectal cancer, lung cancer, breast cancer, nasopharyngeal cancer, oral cancer, esophageal cancer, stomach cancer, bladder cancer, and lymphoma.
